(19) United States
(12) Patent Application Publication
(10) Pub. No.: US 2016/0058814 A1
(43) Pub. Date: Mar. 3, 2016

(54) PLANT EXTRACTS AND RELATED COMPOSITIONS, METHODS AND SYSTEMS

(71) Applicant: Helen Maria MOUSTAKAS, THESSALONIKI (GR)

(72) Inventor: Helen Maria MOUSTAKAS, THESSALONIKI (GR)

(21) Appl. No.: 14/469,129

(22) Filed: Aug. 26, 2014

(51) Int. Cl. A61K 36/54; A61K 36/74; A61K 33/34; A61K 31/375; A61K 31/196; A61K 31/192; A61K 31/07; A61K 31/59; A61K 31/202; A61K 31/216; A61K 31/37; A61K 36/63; A61K 35/60

(52) U.S. Cl. CPC A61K 36/54; A61K 36/63; A61K 36/74; A61K 33/34; A61K 31/375; A61K 31/196; A61K 35/60; A61K 31/07; A61K 31/59; A61K 31/202; A61K 31/216; A61K 31/37; A61K 31/192

(57) ABSTRACT

Ehanolic plant extracts of aerial parts of at least a first plant, a second plant and optionally a third plants are described with related compositions, methods and systems, in which the first plant belongs to plant genus Jasminum and is in flowering stage, the second plant belongs to plant genus Cinnamomum and the third plant belongs to the genus Coffea.

### FIELD

The present disclosure relates to plant extracts and related compositions, methods and systems. In particular, the present disclosure relates to plant extracts and related compositions and systems as well as related methods and uses for various applications comprising medical and pharmacological applications as well as fundamental biology studies.

### BACKGROUND

Natural substances and related use for medical and pharmacological applications, as well as for fundamental biology studies have been the subject of studies and research, in particular when aimed at obtaining effect on biological activities in an individual.

In particular plant extracts have been object of studies to identify active principles to be used to obtain biological activities in individuals.

However, despite various studies and results for candidates or proven active principle from natural substances, such as plants, suitable as active constituents having potency and/or broad spectrum of biological activities remains challenging.

### SUMMARY

Described herein are plant extracts and related formulations, methods and systems. In particular, described herein are ethanolic plant extracts that in several embodiments, can be used in formulation having a biological activity and in particular an ability to affect expression and activity of a substance, such as a protein, that contributes to the cause of a specific biochemical reaction or bodily process (e.g. transcription factors and signaling molecules)

According to a first aspect, an ethanolic plant extract is described. The ethanolic plant extract is obtainable by contacting aerial parts of at least a first plant and a second plant with ethanol with a volume/volume ratio first plant:ethanol from about 1:1.25 to about 1:1.75 and a volume/volume ratio second plant:ethanol from about 1:10 to about 1:18 wherein the first plant belongs to plant genus Jasminum and is in flowering stage, and the second plant belongs to plant genus Cinnamomum. In some embodiments, the aerial parts of at least a first and a second plants further comprise aerial parts of a third plant, and the contacting further comprises contacting the aerial parts of the third plants with ethanol with a volume/volume ratio third plant:ethanol from about 1:25 to about 1:35. In those embodiments, the third plant belongs to the genus Coffea and the aerial parts are green beans of the third plant

According to a second aspect, an ethanolic plant extract is described. The ethanolic plant extract comprises a mixture of alkaloids, phenolic acids and derivatives thereof, polyphenols, terpenes, steroids, methylated phenols; benzopyrans; carbohydrates; free fatty acids and triglycerides, the mixture obtained by performing ethanol extraction of aerial parts of at least a first plant and a second plant, wherein the first plant belongs to plant genus Jasminum, and the second plant belongs to plant genus Cinnamomum In some embodiments, the aerial parts of at least a first plant and a second plant further comprise aerial parts of a third plant wherein the third plant belongs to the genus Coffea

According to a third aspect, a formulation is disclosed. The formulation comprises one or more ethanolic plant extracts herein described, and at least one additional active agent selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid (DHA) or a combination thereof.

According to a fourth aspect, use of a formulation herein described to elicit a biological response in a biological environment, and a related method are described. The method comprises contacting the biological environment with an amount of a formulation herein described comprising all the additional active agents in an effective amount to increase activation of transcription factor NF-kB, increase expression of NGF (Nerve Growth Factor); increasing the biological activity of cholinergic synapses; decreasing the phosphorylation of Tau protein; and/or reduce gene expression of Htr1b gene (receptor 5-hydroxytryptamine or serotonin receptor 1B).

According to a fifth aspect, use of a formulation herein described to treat an individual, and are a method are described. The method comprises administering to the individual an amount of a formulation herein described comprising all the additional active agents in an effective amount to increase activation of transcription factor NF-kB, increase expression of NGF (Nerve Growth Factor); increasing the biological activity of cholinergic synapses; decreasing the phosphorylation of Tau protein; and/or reduce gene expression of Htr1b gene (receptor 5-hydroxytryptamine or serotonin receptor 1B).

According to a fifth aspect, use of extract herein described for providing a plant extract capable of eliciting a biological effect in an individual and a related method are described. The method comprises contacting an aerial part of at least a first plant and a second plant with ethanol with a molar ratio first plant:ethanol from about 1:1.25 to about 1:1.75 and a molar ratio second plant:ethanol from about 1:10 to about 1:18 wherein the first plant belongs to plant genus Jasminum and is in flowering stage, and the second plant belongs to plant genus Cinnamomum. In some embodiments, the aerial parts of at least a first and a second plants further comprise aerial parts of a third plant, and the contacting further comprises contacting the aerial parts of the third plants with ethanol with a molar ratio third plant:ethanol from about 1:25 to about 1:35. In those embodiments, the third plant belongs to the genus Coffea.

According to a sixth aspect, a method and use of an ethanolic plant extract to provide a formulation capable of eliciting a biological response in an individual, are described. The method comprises providing one or more ethanolic plant extracts herein described; adding to the one or more ethanolic plant extracts at least one active agent to provide a candidate formulation; and testing the candidate formulation to detect a biological activity in vitro or in vivo. In some embodiments the at least one active agents comprise at least one substance selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid DHA or a combination thereof and the candidate formulation is a formulation herein described comprising at least additional active agent herein described.

According to a seventh aspect, a method and use of a biologically active agent to provide a formulation capable of eliciting a biological response in an individual, is described. The method comprises providing at least one active agent selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid (DHA) or a combination thereof; adding to the at least one active agent to an ethanolic plant extract to provide a candidate formulation; and testing the candidate formulation to detect a biological activity in vitro or in vivo.

According to an eight aspect, a system to provide a formulation having a biological activity, is disclosed. The system comprises one or more ethanolic plant extracts herein described, and at least one biologically active agents selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid DHA or a combination thereof, for combined use in providing a formulation having a biological activity in accordance with the disclosure.

The ethanolic plant extracts, uses, compositions, methods, and systems herein described can be used in connection with applications wherein elicitation of a biological activity in a system and in particular a biological system is desired. In particular in accordance with some embodiments, ethanolic plant extracts, active agents and related formulations herein described can be used in applications wherein to increase activation of transcription factor NF-kB, increase expression of NGF (Nerve Growth Factor); increasing the biological activity of cholinergic synapses; decreasing the phosphorylation of Tau protein; and/or reduce gene expression of Htr1b gene (receptor 5-hydroxytryptamine or serotonin receptor 1B), is desired. Exemplary applications comprise medical, pharmaceutical, veterinary applications as well as fundamental biological studies and various applications, identifiable by a skilled person upon reading of the present disclosure, wherein triggering of one or more biological activity in a biological system is desired.

The details of one or more embodiments of the disclosure are set forth in the accompanying drawings and the description below. Other features and objects will be apparent from the description and drawings, and from the claims.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 shows a GC-MS chromatogram of EMM extract before evaporation.

FIG. 2 shows a mass spectrum of the major metabolite of EMM extract.

FIG. 3 shows a GC-MS chromatogram of EMM extract before evaporation.

FIG. 4 shows a GC-MS chromatogram of EMM extract after evaporation.

FIG. 5 shows a GC-MS chromatogram of fraction EMMA.

FIG. 6 shows a mass spectrum of the major metabolite (cinnamic aldehyde).

FIG. 7 shows a ¹H-NMR spectrum of EMM in MeOH-d4.

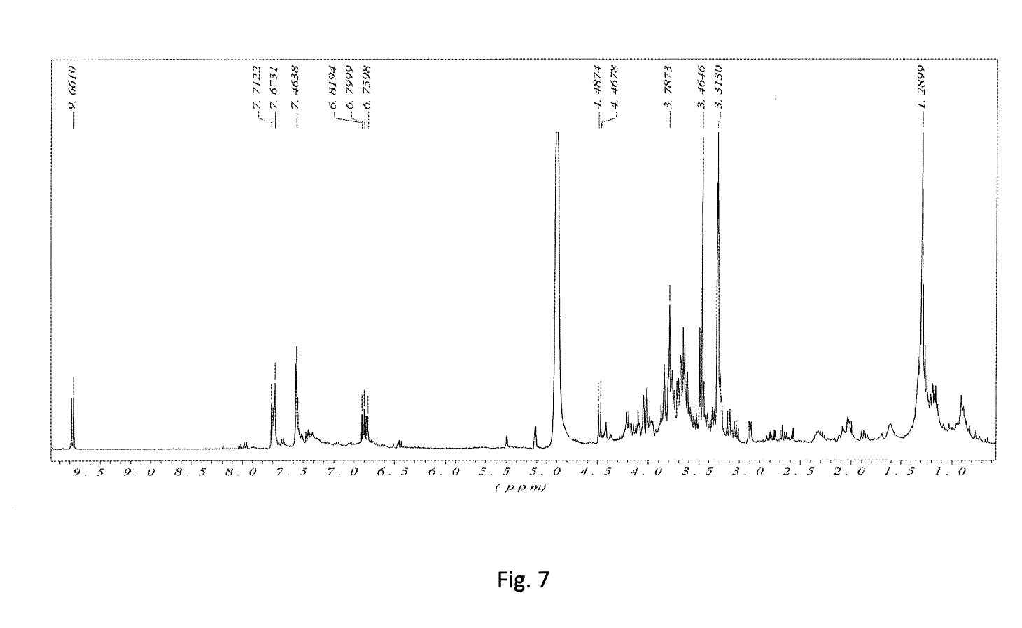

FIG. 8 shows a ¹H-NMR spectrum of EMMB in MeOH-d4.

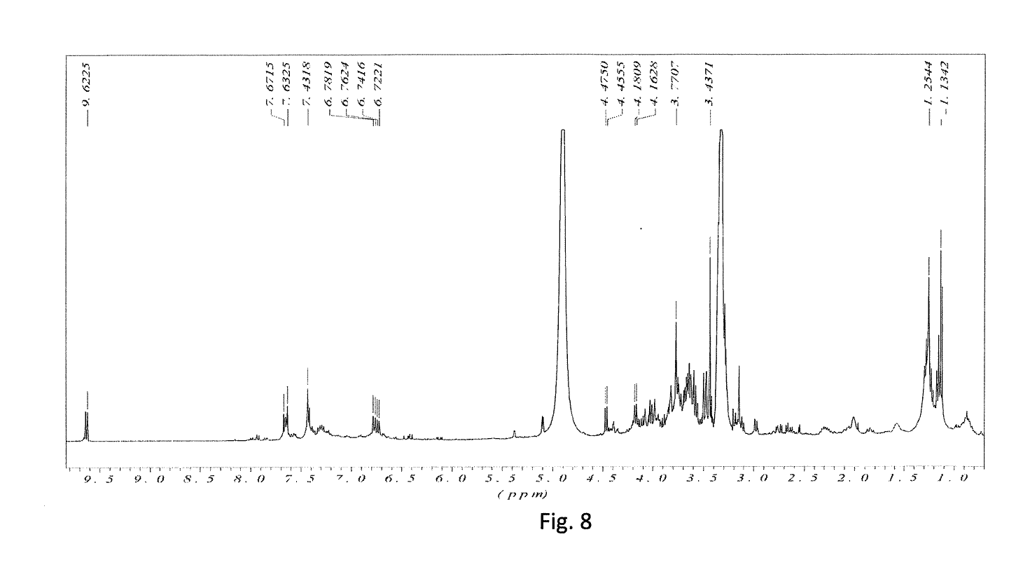

FIG. 9 shows a ¹H-NMR spectrum of EMMB1 in CDCl₃.

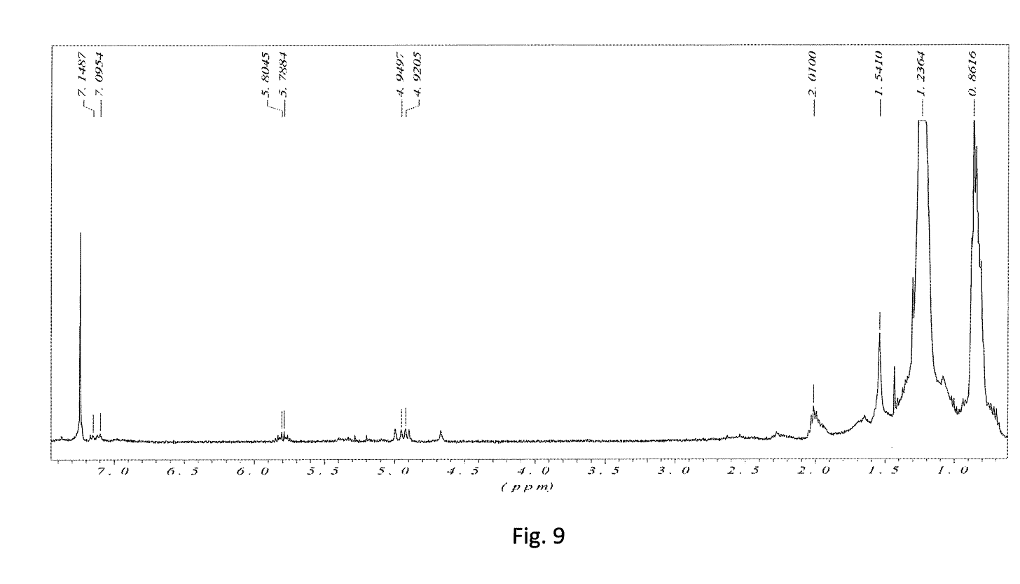

FIG. 10 shows a ¹H-NMR spectrum of EMMB2 in CDCl₃.

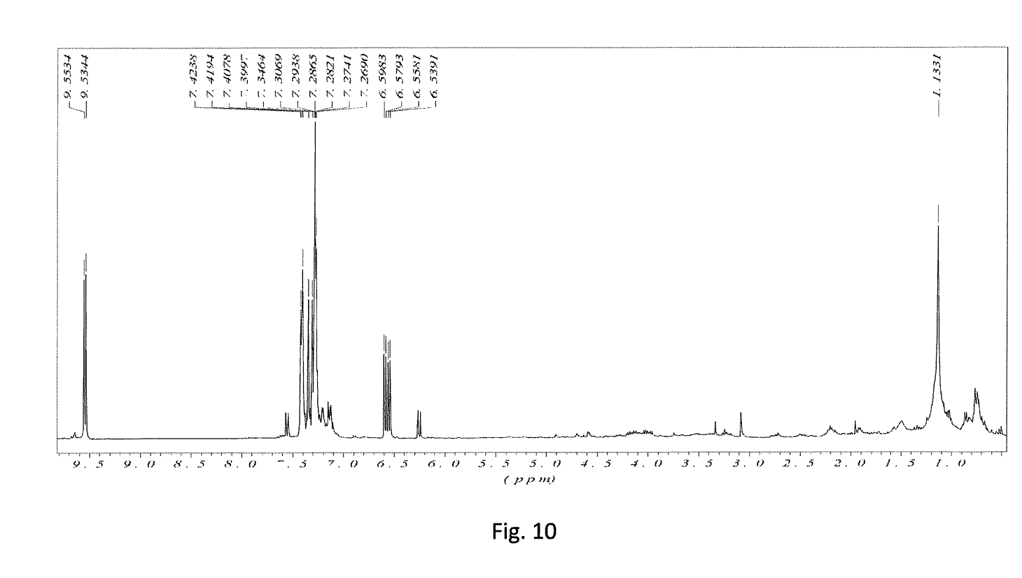

FIG. 11 shows a ¹H-NMR spectrum of EMMB3 in CDCl₃.

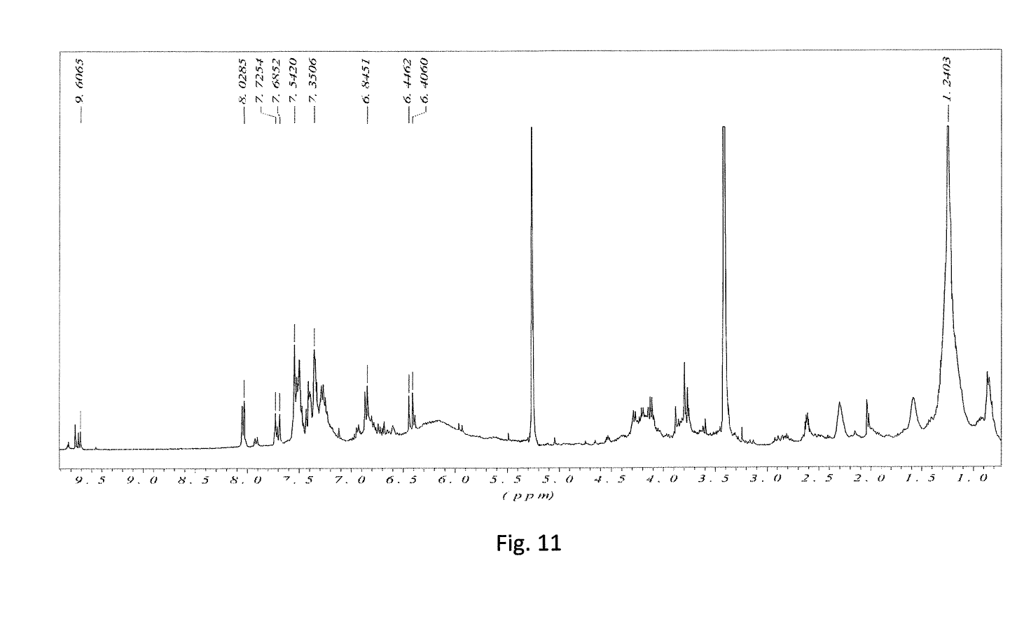

FIG. 12 shows a ¹H-NMR spectrum of EMMB4 in MeOH-d4.

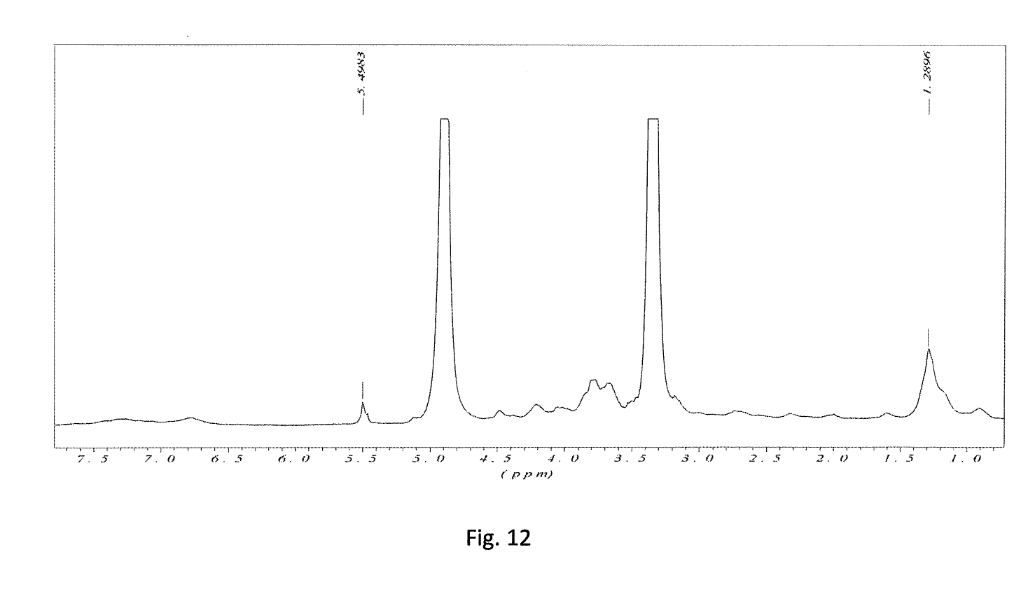

FIG. 13 shows a ¹H-NMR spectrum of EMMB5 in MeOH-d4.

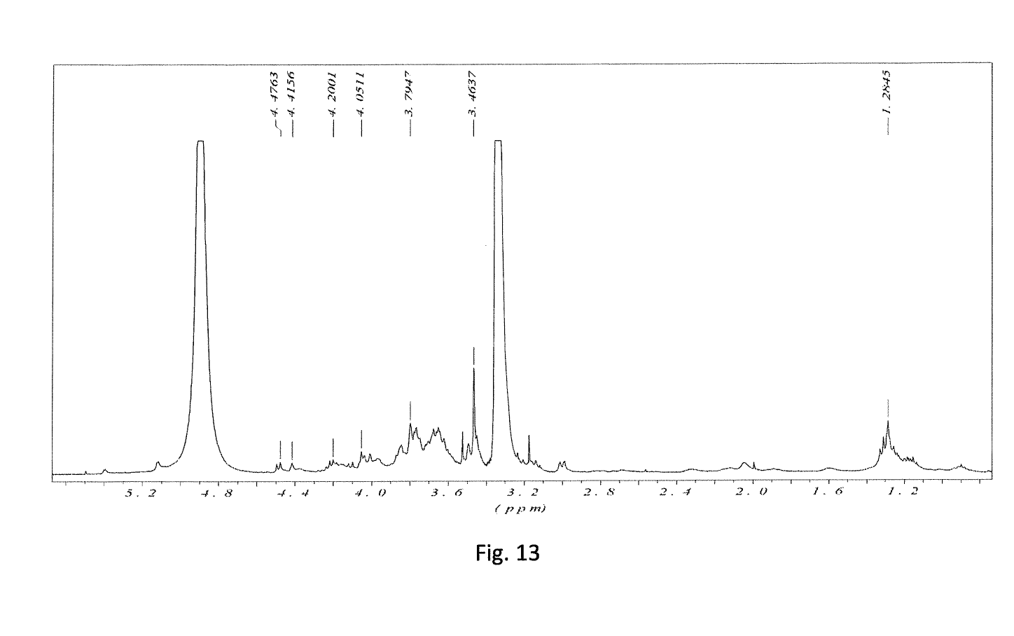

FIG. 14 shows a ¹H-NMR spectrum of EMMS1 in CDCl₃ (+3 drops of MeOH-d4).

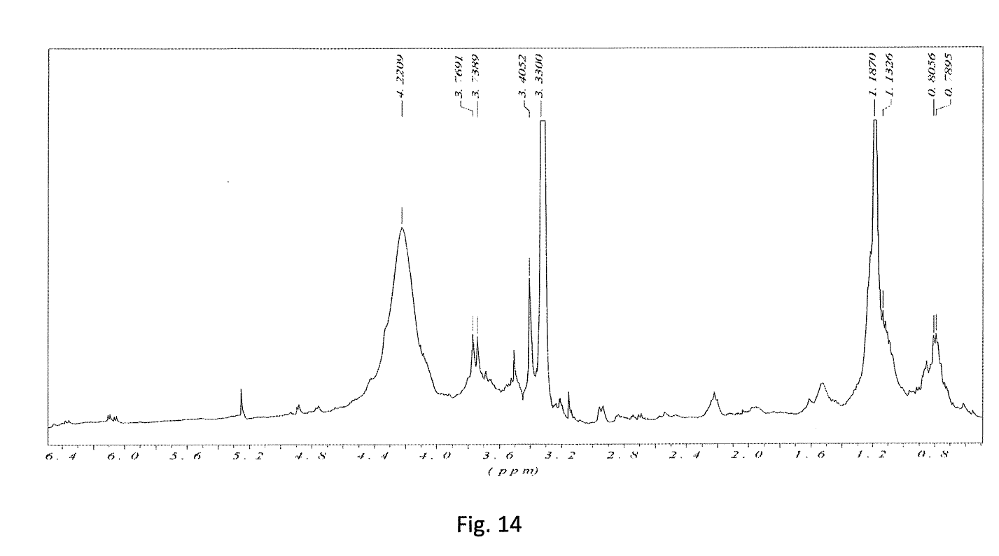

FIG. 15 shows a ¹H-NMR spectrum of EMMS2 in MeOH-d4.

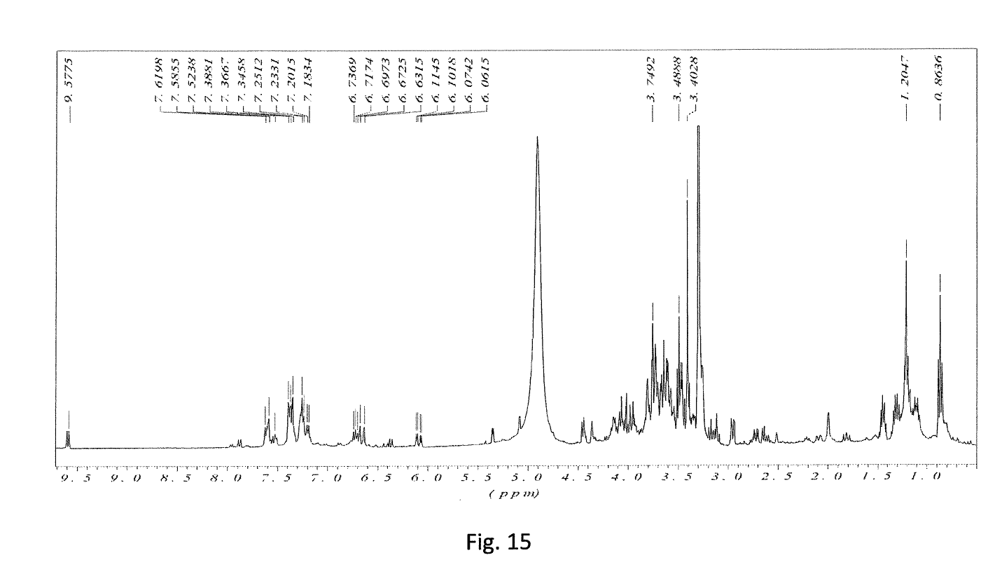

FIG. 16 shows a ¹H-NMR spectrum of EMMS3 in MeOH-d4.

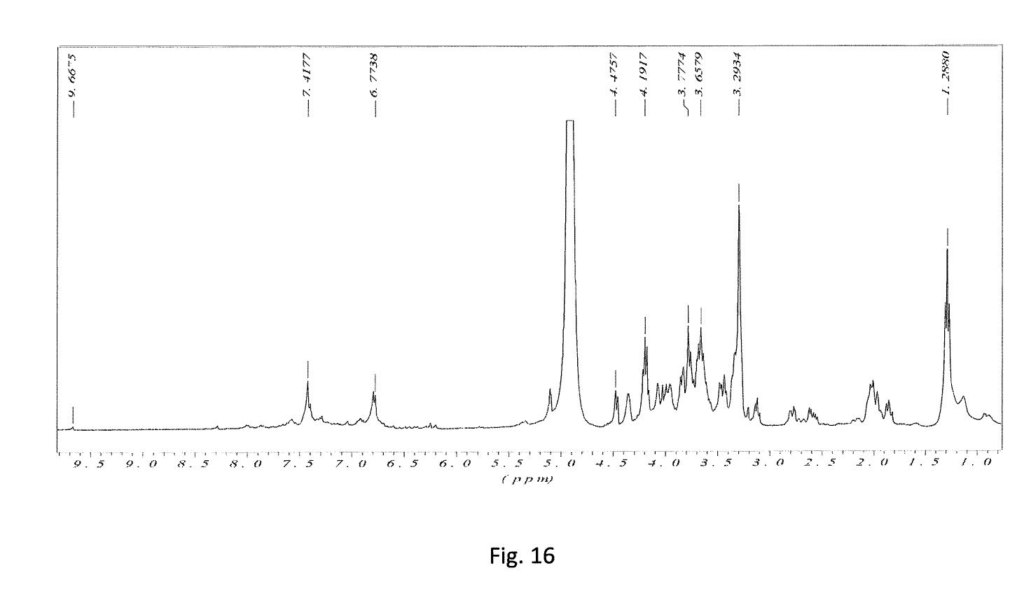

FIG. 17 shows a ¹H-NMR spectrum of EMMR1 in MeOH-d4.

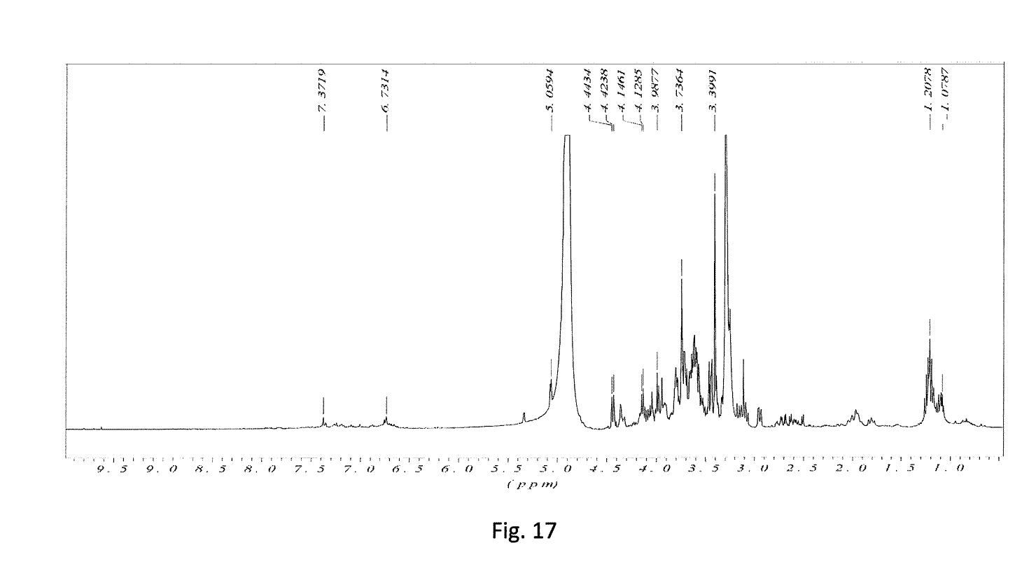

FIG. 18 shows a ¹H-NMR spectrum of EMMR2 in MeOH-d4.

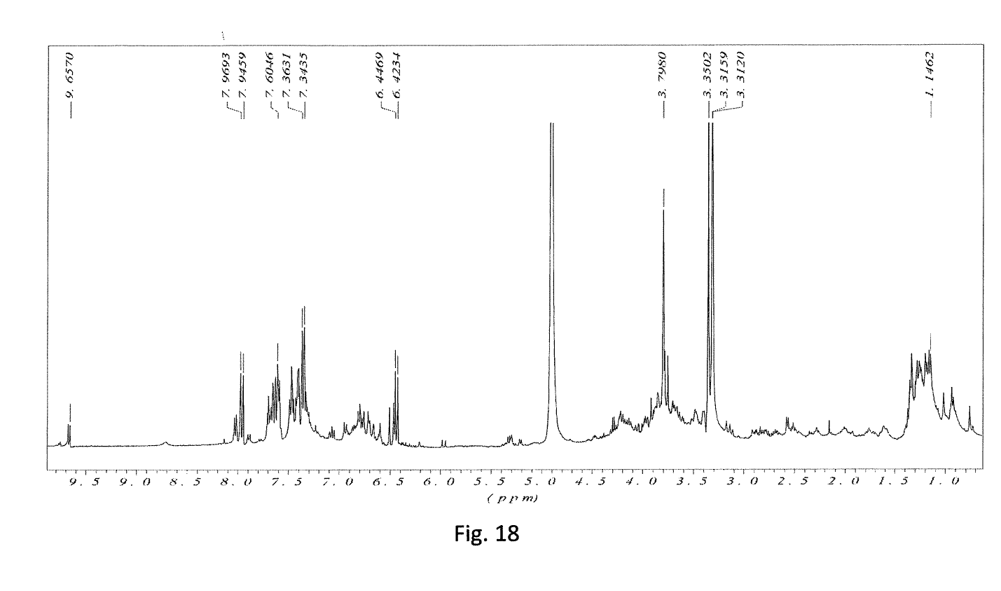

FIG. 19 shows ¹H-NMR spectrum of EMMR3 in MeOH-d4.

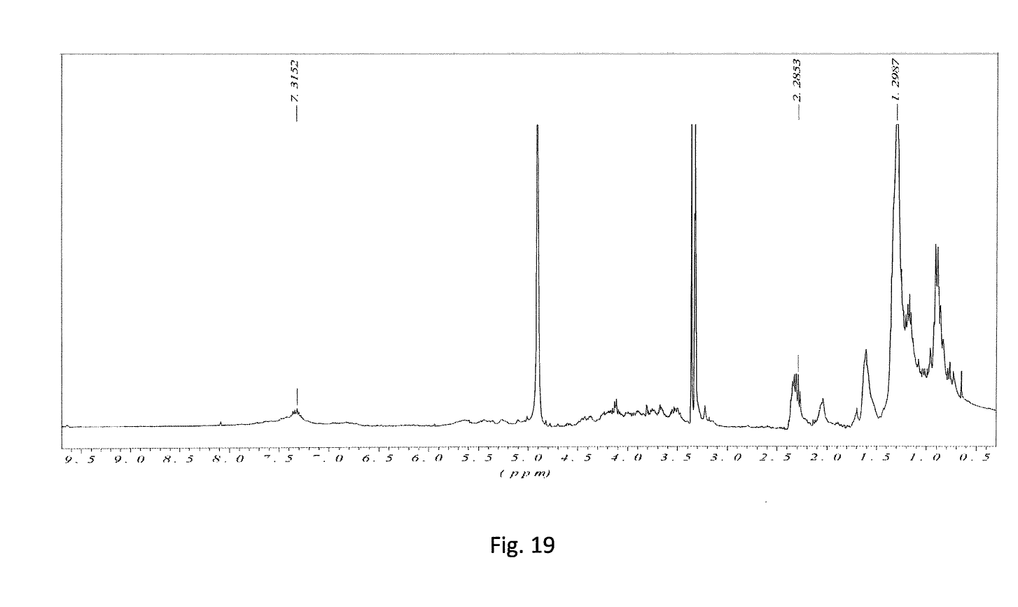

FIG. 20 shows a schematic procedure based on extraction with two-phase liquid systems.

FIG. 21 shows a ¹H-NMR spectrum of EMMN_{O}3 in MeOH-d4.

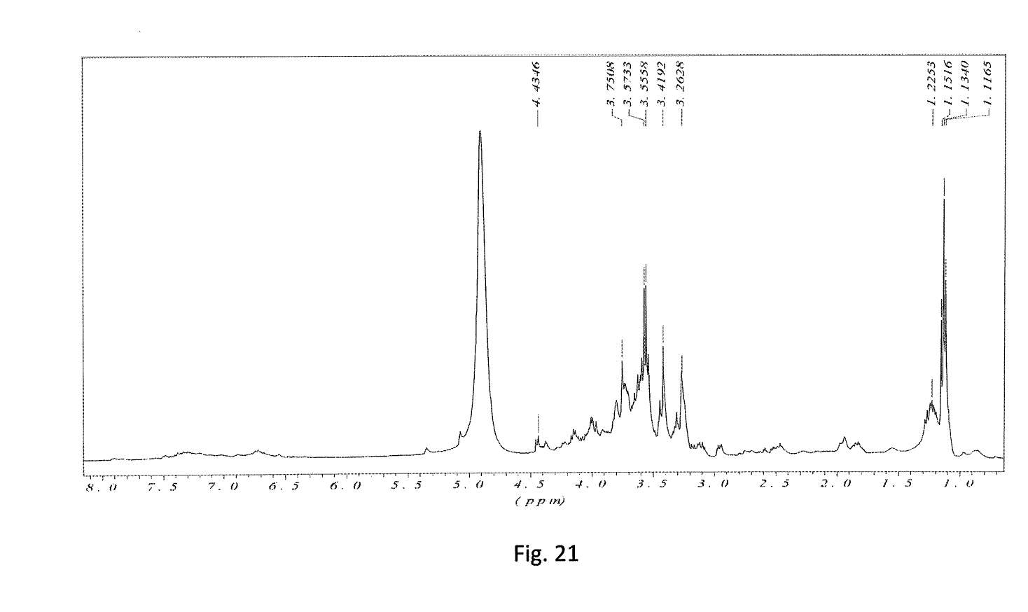

FIG. 22 shows a schematic procedure of successive partitioning with two-phase liquid systems to yield five fractions (EMML1-EMML5) which contain metabolites of different polarities.

FIG. 23 shows a ¹H-NMR spectrum of EMML2 in CDCl₃.

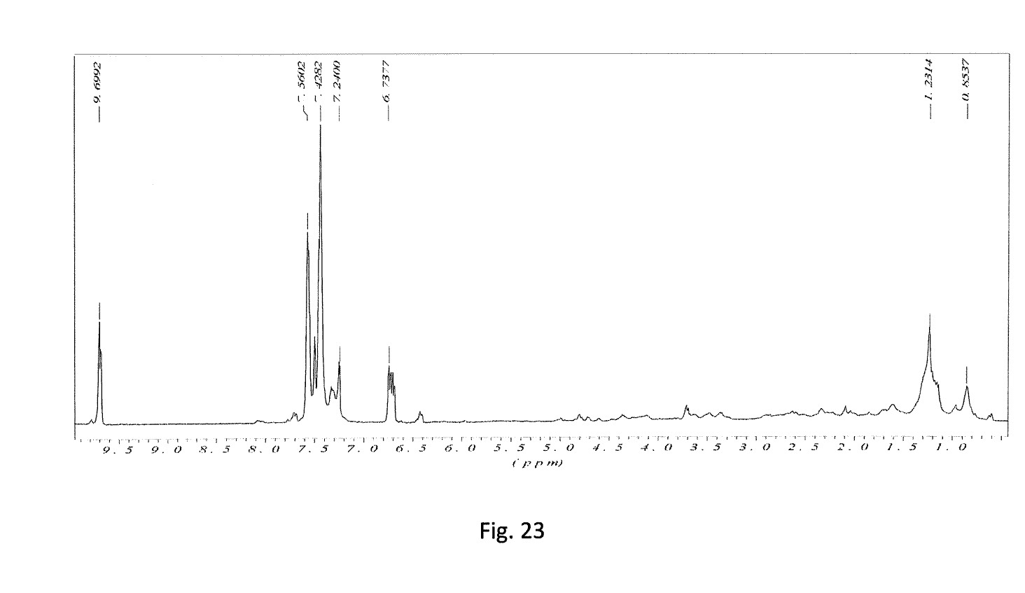

FIG. 24 shows a schematic procedure to partition a plant extract with two-phase liquid systems to yield five fractions (RJ1-RJ5) which contain metabolites of different polarities.

FIG. 25 shows a ¹H-NMR spectrum of Plant 2 in MeOH-d4.

FIG. 26 shows a GC-MS chromatogram of EMMN extract of coumarin.

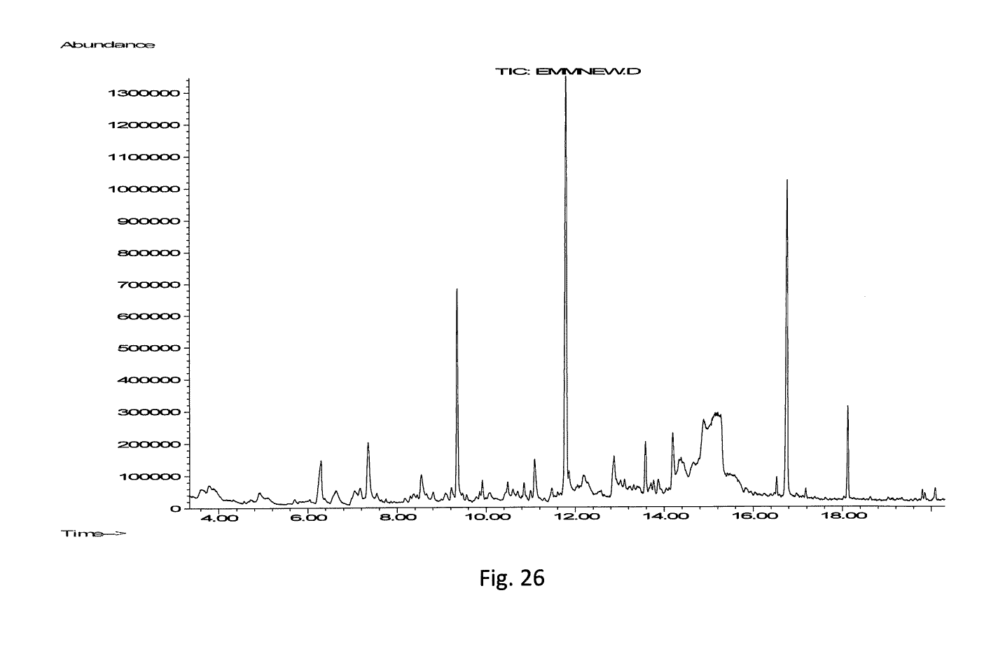

FIG. 27 shows a mass spectrum of EMMN extract of coumarin.

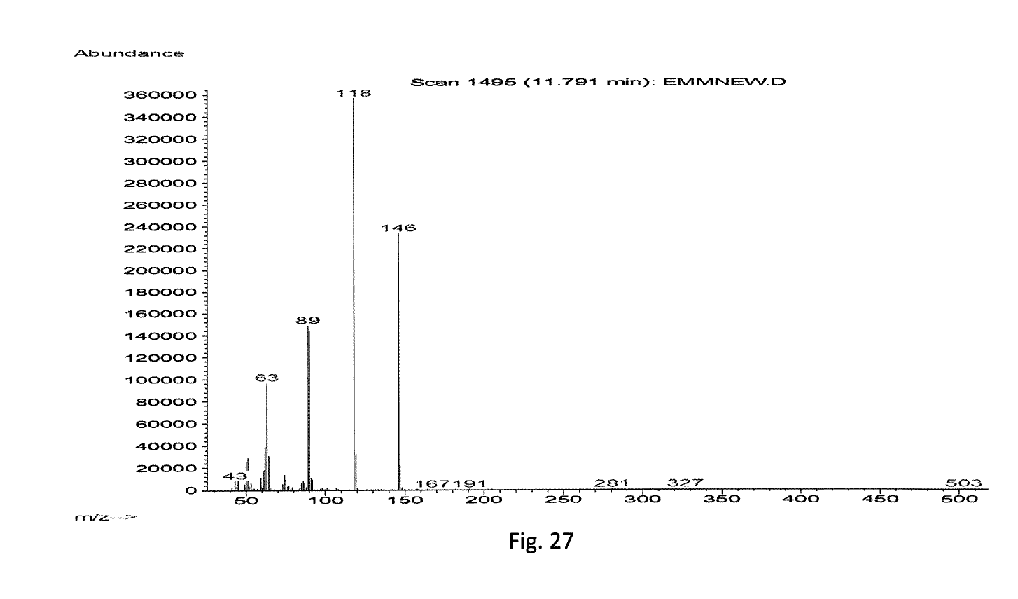

FIG. 28 shows a GC-MS chromatogram of EMMN extract co-chromatographed with scopoletin.

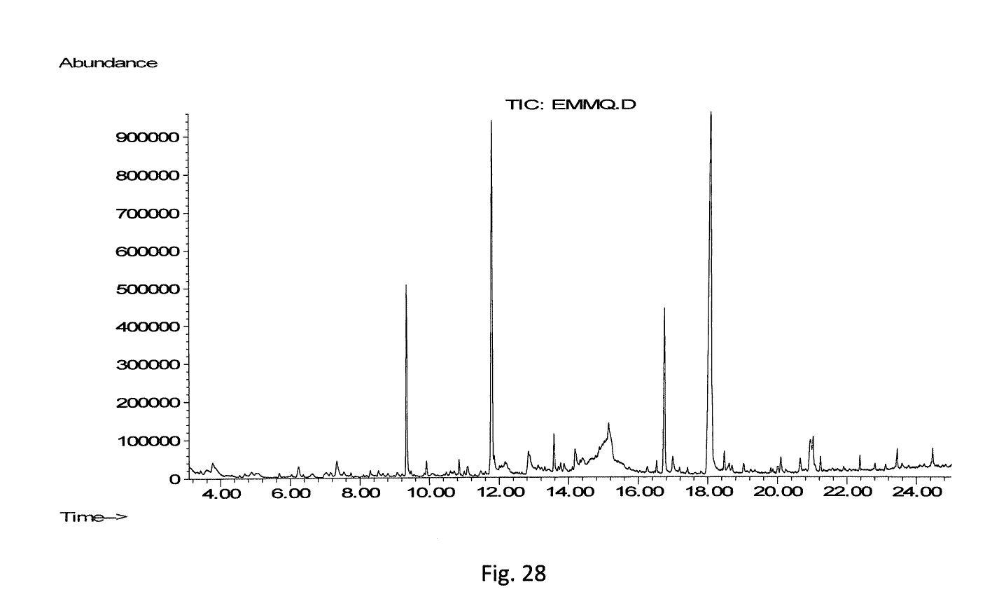

FIG. 29 shows a mass spectrum of scopoletin.

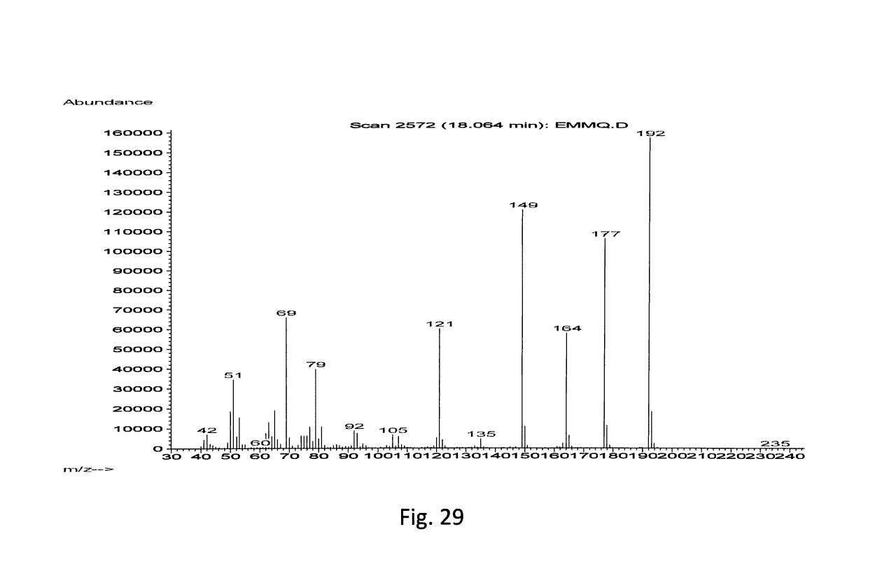

FIG. 30 shows a HPLC-DAD chromatogram (at 280 nm) of EMMN extract.

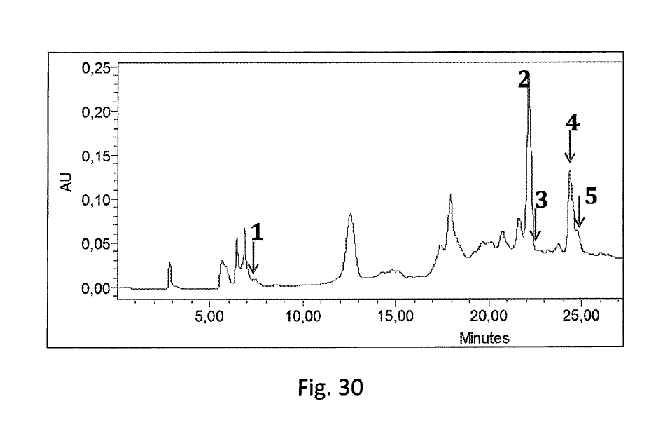

FIG. 31 shows a ¹H-NMR spectrum of EMMN extract in MeOH-d4.

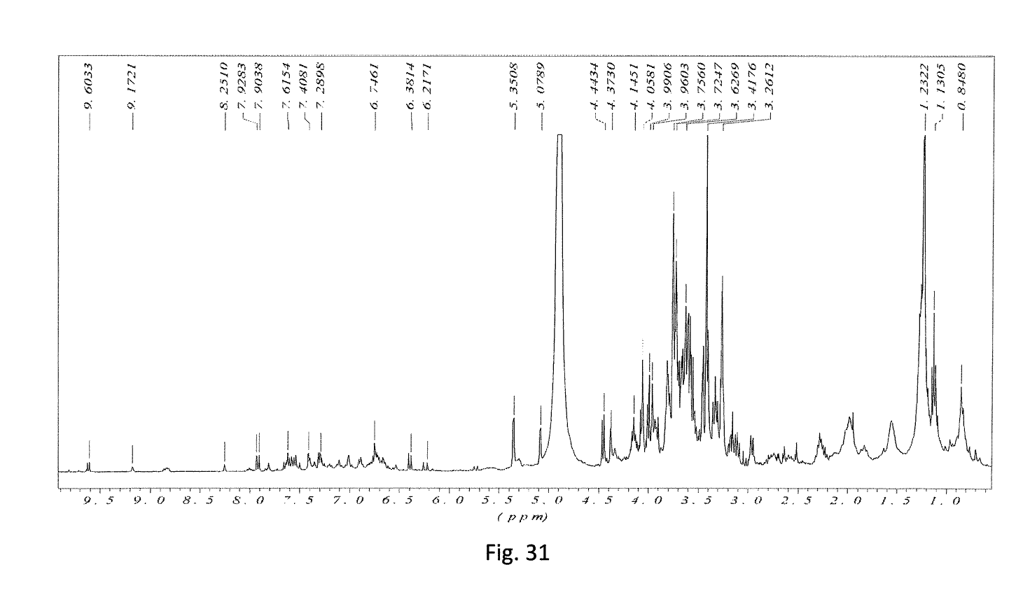

FIG. 32 shows percentage % activation of the transcription factor NF-kB (p<0.001).

FIG. 33 shows percentage % activation of the transcription factor NF-kB (p<0.001).

FIG. 34 shows immunoprecipitation of the complex based TRAFsfml-TRAF6 induced by activated cytoplasmic domain of the receptor CD40;

FIG. 35 shows immunoprecipitation of the complex based TRAFsfml-TRAF6 induced by activated cytoplasmic domain of the receptor CD40.

FIG. 36 shows the results of concentration of antibodies against thymus DNA (OD).

FIG. 37 shows kidney sections and detection of immunoglobulins IgG mouse with fluorescence.

FIG. 38 shows sections of liver of transgenic mice LMP1/CD40 hematoxylin-eosin staining.

FIG. 39 shows. an electrophoresis gel illustrating the results of immunoprecipitation in a mouse model showing production of NGF factor following administration of an extract according to embodiments herein described.

FIG. 40 shows percentage % activation of the transcription factor NF-kB (p<0.001).

FIG. 41 shows percentage % activation of the transcription factor NF-kB (p<0.001).

FIG. 42 shows immunoprecipitation of the complex based TRAFsfml-TRAF6 induced by activated cytoplasmic domain of the receptor CD40.

FIG. 43 shows immunoprecipitation of the complex based TRAFsfml-TRAF6 induced by activated cytoplasmic domain of the CD40 receptor.

FIG. 44 shows concentration of antibodies against thymus DNA (OD).

FIG. 45 shows kidney sections and detection of immunoglobulins IgG mouse with fluorescence.

FIG. 46 shows sections of liver of transgenic mice LMP1/CD40 hematoxylin-eosin stainin.

FIG. 47 shows kidney sections and detection of immunoglobulins IgG mouse with fluorescence.

### DETAILED DESCRIPTION

Described herein are plant extracts and related formulations, methods, and uses that in some embodiments determine an effect on biological activity in an individual.

The term “plant” as used herein refers to are living multicellular organisms of the kingdom Plantae (Viridiplantae in Latin). Plants form a clade that includes flowering plants, gymnosperms, ferns, clubmosses, hornworts, liverworts and mosses. In particular, plants in the sense of the disclosure relates to seed producing plants and more particular to gymnosperm and flowering plants to also known as Angiospermae or Magnoliophyta, which are the most diverse group of land plants. The term “gymnosperm” indicates seed producing plants named after the unenclosed or “naked” condition of their seeds (called ovules in their unfertilized state). The term “angiosperm” indicates a seed-producing plant that can be distinguished from the gymnosperms by a series of characteristic such as flowers, endosperm within the seeds, production of fruits that contain the seeds, and having seeds and ovules enclosed within an ovary as will be understood by a skilled person.

According to the present disclosure, a plant generically includes aerial parts and roots wherein aerial parts of the plants indicates all those parts which are in contact with air and can typically be seen directly from eye (those that lie in air). Aerial parts of a plant sometimes also called shoot relate to stem, leaves, flowers and fruit and seeds. Roots instead in the sense of the present disclosure indicate the organ of a plant that typically lies below the surface of the soil. Accordingly aerial parts of a plant comprise plant fruits, plant leaves, plant branches, plant seeds e.g. plant beans, plant sticks, plant flowers.

A “plant” in the sense of the present disclosure has a life cycle which typically includes germination, growth, sprouting, fruit production where fruits comprise seeds. In particular, the life cycle of a plant starts when the seed germinates into a seedling. This seedling will then grow into an adult plant, which in case of flowering plants will sprout into flowers. The flowers will produce fruits which contain seeds.

In embodiments herein described, an ethanolic plant extracts are provided together with related formulations, methods and systems.

The term ‘extraction” refers to a separation process consisting in the separation of one or more analytes from the components of a sample other than the one or more analytes. Extractions are processes that typically use two immiscible phases to separate one or more solutes from one phase into the other. The distribution of a solute between two phases is an equilibrium condition described by partition theory. For example, boiling tea leaves in water extracts the tannins, theobromine, and caffeine out of the leaves and into the water. More typical extractions preformed typically but not only in a laboratory settings are of organic compounds out of an aqueous phase and into an organic phase. Common extractants are arranged from ethyl acetate to water (ethyl acetate<acetone<ethanol<methanol<acetone:water (7:3)<ethanol:water (8:2)<methanol:water (8:2)<water) in increasing order of polarity according to the Hildebrand solubility parameter.

The term “extract” as used herein refers to the result of such process of separation that can take the form of a solution formulation or other chemical form depending on the extraction process. In particular, the term extract can relate to a substance made by extracting a part of a sample (e.g. a raw material), by using a solvent such as ethanol or water. In various instances extract relates to a solvent that is enriched in one or more solute. In particular, a “plant extract” in the sense of the present disclosure typically comprises a concentrated preparation of a plant material obtained by isolating or purifying desired active constituents with one or more extraction processes.

In embodiments herein described the plant extracts are ethanolic plant extracts. The term “ethanol” as used herein also called ethyl alcohol, pure alcohol, grain alcohol, or drinking alcohol, refers to a volatile, flammable, colorless liquid with the structural formula CH₃CH₂OH, often abbreviated as C₂H₅OH or C₂H₆O. The adjective “ethanolic” as used herein refers to also any solution or formulation containing ethanol or to a process wherein ethanol is used as will be understood by a skilled person based on the context where the term is used.

The expression “ethanolic plant extract” as used herein refers to the results of an extraction performed using ethanol as a solvent on one or more plant parts. In particular A solvent is a substance that dissolves a solute (a chemically different liquid, solid or gas), resulting in a solution. The quantity of solute that can dissolve in a specific volume of solvent varies with temperature and with the solvent as will be understood by a skilled person. Ethanol is a versatile solvent, miscible with water and with many organic solvents, including acetic acid, acetone, benzene, carbon tetrachloride, chloroform, diethyl ether, ethylene glycol, glycerol, nitromethane, pyridine, and toluene. Ethanol is also miscible with light aliphatic hydrocarbons, such as pentane and hexane, and with aliphatic chlorides such as trichloroethane and tetrachloroethylene. Ethanol's miscibility with water contrasts with the immiscibility of longer-chain alcohols (five or more carbon atoms), whose water miscibility decreases sharply as the number of carbons increases. The miscibility of ethanol with alkanes is limited to alkanes up to undecane, mixtures with dodecane and higher alkanes show a miscibility gap below a certain temperature (about 13° C. for dodecane). The miscibility gap tends to get wider with higher alkanes and the temperature for complete miscibility increases. Ethanol-water mixtures have less volume than the sum of their individual components at the given fractions. Mixing equal volumes of ethanol and water results in only 1.92 volumes of mixture. Mixtures of ethanol and water form an azeotrope at about 89 mole-% ethanol and 11 mole-% water or a mixture of about 96 volume percent ethanol and 4% water at normal pressure and T=351 K. This azeotropic composition is strongly temperature- and pressure-dependent and vanishes at temperatures below 303 K. The term “ethanol 90” “ethanol 95” and “ethanol 99” as used herein refers to also pure ethanol, 90%, 95% and 99% in volume respectively wherein the balance is provided by water such as the ones commercially available. The polar nature of the hydroxyl group causes ethanol to dissolve many ionic compounds, notably sodium and potassium hydroxides, magnesium chloride, calcium chloride, ammonium chloride, ammonium bromide, and sodium bromide. Sodium and potassium chlorides are slightly soluble in ethanol. Because the ethanol molecule also has a nonpolar endethanol is also capable of dissolving nonpolar substances, including most essential oils and numerous flavoring, coloring, and medicinal agents.

In embodiments herein described ethanol extraction is performed by contacting parts of one or more plant with ethanol or ethanolic solution in order to separate plant substances soluble in ethanol from the plant. The one or more substances are then contained in the ethanolic solution. The substance or substances of the plant remain in the ethanolic solution when the plant is removed from the ethanolic solution.

In some embodiments, the parts of the plants are aerial parts of a first plant and a second plant wherein the first plant belongs to the genus Jasminum and the second plant belong to the genus Cinnamomum

The term “Jasminum” as used herein indicates a genus of flowering plants and in particular shrubs and vines in the olive family (Oleaceae). It contains around 200 species native to tropical and warm temperate regions of Europe, Asia, and Africa. Jasmines are widely cultivated for the characteristic fragrance of their flowers. In particular, Species belonging to genus Jasminum are classified under the tribe Jasmineae of the olive family (Oleaceae). Jasminum is divided into five sections—Altemifolia, Jasminum, Primulina, Trifoliolata, and Unifoliolata. Exemplary species comprise Jasminum abyssinicum Hochst. ex DC. (forest jasmine), Jasminum adenophyllum Wall. (bluegrape jasmine, pinwheel jasmine, princess jasmine), Jasminum angulare Vahl, Jasminum angustifolium (L.) Willd., Jasminum auriculatum Vahl, (Indian jasmine, needle-flower jasmine) Jasminum azoricum L., Jasminum beesianum Forrest & Diels (red jasmine), Jasminum dichotomum Vahl (Gold Coast jasmine) Jasminum didymum G. Forst., Jasminum dispermum Wall., Jasminum elegans Knobl., Jasminum elongatum (P. J. Bergius) Willd. J. floridum Bunge, Jasminum fluminense Vell., Jasminum fruticans L., Jasminum grandiflorum L. (Catalonian jasmine, jasmin odorant, royal jasmine, Spanish jasmine), Jasminum humile L. (Italian jasmine, Italian yellow jasmine), Jasminum anceolarium Roxb., Jasminum mesnyi Hance (Japanese jasmine, primrose jasmine, yellow jasmine), Jasminum multiflorum (Burm. f.) Andrews (Indian jasmine, star jasmine, winter jasmine), Jasminum multipartitum Hochst (starry wild jasmine), Jasminum nervosum Lour., Jasminum nobile C. B. Clarke, Jasminum nudiflorum Lindl. (winter jasmine), Jasminum odoratissimum L. (yellow jasmine), Jasminum parkeri Dunn (dwarf jasmine), Jasminum polyanthum Franch., Jasminum sambac (L.) Aiton (Arabian jasmine, Sambac jasmine), Jasminum simplicifolium G. Forst., Jasminum sinense Hemsl., Jasminum subhumile W. W. Sm. Jasminum subtriplinerve Blume, Jasminum tortuosum Willd. and JASMINUM urophyllum Hem. A representative plant belonging to the genus Jasminum is common Jasmine (Jasminum officinale L. also known as, jasmine, jessamine, poet's jasmine, summer jasmine, white jasmine). In particular, according to an embodiment, the plant belonging to the genus Jasminum can be the Jasminum officinale. Processes and methods herein described with Jasminum officinale allow to obtain extracts such as the extracts shown in the examples. According to some embodiments of the present disclosure, the plant belonging to the genus Jasminum is any one of the above-reported jasminum species. Processes and methods herein described with Jasminum officinale can be performed with any one of the above jasminum species and are expected to result in efficacious extracts in accordance with the disclosure.

The term “Cinnamomum” as used herein indicates a genus of flowering plants and in particular evergreen aromatic trees and shrubs belonging to the laurel family, Lauraceae. The species of Cinnamomum have aromatic oils in their leaves and bark. The genus contains over 300 species, distributed in tropical and subtropical regions of North America, Central America, South America, Asia, Oceania, and Australasia. Exemplary species of the genus cinnamomum comprise Cinnamomum acuminatifolium, Cinnamomum acuminatis simum, Cinnamomum acutatum, Cinnamomum africanum, Cinnamomum aggregatum, Cinnamomum alainii, Cinnamomum alatum, Cinnamomum albiflorum, Cinnamomum alcinii, Cinnamomum alexei, Cinnamomum alibertii, Cinnamomum alternifolium, Cinnamomum altis simum, Cinnamomum ammannii, Cinnamomum amoenum, Cinnamomum amplexicaule, Cinnamomum amplifolium, Cinnamomum anacardium, Cinnamomum andersonii, Cinnamomum angustifolium, Cinnamomum angustitepalum, Cinnamomum antillarum, Cinnamomum appelianum, Cinnamomum arbusculum, Cinnamomum archboldianum, Cinnamomum areolatocostae, Cinnamomum areolatum, Cinnamomum arfakense, Cinnamomum argenteum, Cinnamomum aromaticum-cassia, Cinnamomum arsenei, Cinnamomum asa-grayi, Cinnamomum assamicum, Cinnamomum aubletii, Cinnamomum aureo-fulvum, Cinnamomum austral, Cinnamomum austro-sinense, Cinnamomum austro-yunnanense, Cinnamomum bahianum, Cinnamomum bahiense, Cinnamomum baileyanum, Cinnamomum baillonii, Cinnamomum balansae, Cinnamomum bamoense, Cinnamomum barbato-axillatum, Cinnamomum barbeyanum, Cinnamomum barlowii, Cinnamomum bartheifolium, Cinnamomum barthii, Cinnamomum bazania, Cinnamomum beccarii, Cinnamomum bejolghota, Cinnamomum bengalense, Cinnamomum biafranum, Cinnamomum bintulense, Cinnamomum birmanicum, Cinnamomum blumei, Cinnamomum bodinieri, Cinnamomum bonii, Cinnamomum bonplandii, Cinnamomum borneense, Cinnamomum bourgeauvianum, Cinnamomum boutonii, Cinnamomum brachythyrsum, Cinnamomum bractefoliaceum, Cinnamomum burmannii-Indonesian cinnamon, Cinnamomum camphora-camphor laurel, Cinnamomum cassia-(C. aromaticum), Cinnamomum caudiferum, Cinnamomum chartophyllum, Cinnamomum citriodorum-Malabar cinnamon, Cinnamomum contractum, Cinnamomum filipes, Cinnamomum glanduliferum, Cinnamomum glaucescens, Cinnamomum ilicioides, Cinnamomum impressinervium, Cinnamomum iners, Cinnamomum japonicum-(C. pedunculatum Japanese cinnamon), Cinnamomum javanicum, Cinnamomum jensenianum, Cinnamomum kanehirae-(stout camphor tree niu zhang endemic to Taiwan), Cinnamomum kotoense, Cinnamomum kwangtungense, Cinnamomum liangii, Cinnamomum longepaniculatum, Cinnamomum longipetiolatum, Cinnamomum loureiroi—(Saigon cinnamon), Cinnamomum mairei, Cinnamomum micranthum, Cinnamomum migao, Cinnamomum mercadoi Vidal—(kalingag tree), Cinnamomum mollifolium, Cinnamomum oliveri, Cinnamomum osmophloeum-(pseudocinnamomum), Cinnamomum parthenoxylon-(Selasian wood, Martaban camphor wood, saffrol laurel, alcanforero amarillo, mreah prew phnom, kayu gadis, telasihan, huang zhang, Leaves of Cinnamomum parthenoxylon, Cinnamomum pauciflorum, Cinnamomum philippinense, Cinnamomum pingbienense, Cinnamomum pittosporoides, Cinnamomum platyphyllum, Cinnamomum porphyrium, Cinnamomum porrectum, Cinnamomum reticulatum, Cinnamomum rigidissimum, Cinnamomum saxatile, Cinnamomum septentrionale, Cinnamomum sintoc Blume, Cinnamomum subavenium, Cinnamomum tamala-(tejpat, Indian bay leaf, or malabathrum), Cinnamomum tenuipilum, Cinnamomum tonkinense, Cinnamomum triplinerve, Cinnamomum tsangii, Cinnamomum tsoi, Cinnamomum validinerve, Cinnamomum virens-(red-barked sassafras, eastern Australia), Cinnamomum wilsonii. A representative species of cinnamonum is Cinnamomum verum (cinnamon, Ceylon cinnamon, or true cinnamon). In particular, according to some embodiments, the plant belonging to the genus cinnamon can be Cinnamomum verum. Exemplary methods and process herein described performed with Cinnamomum verum allow obtainment of efficacious extracts in accordance with the present disclosure such as the extracts described in the examples section. According to some embodiments of the present disclosure, the plant belonging to the genus Cinnamomum can be any one of the above-reported cinnamomum species Processes and methods herein described with Cinnamomum Verum can be performed with any one of the above jasminum species and are expected to result in efficacious extracts in accordance with the disclosure.

In particular in embodiments herein described the ethanol extract is obtained by contacting aerial parts of a first plant belonging to the genus of Jasminum in a flowering stage and a from a second plant belonging to the genus of Cinnamomum with ethanol selected between ethanol 90% to ethanol 99% to obtain an extract comprising alkaloids, phenolic acids and derivatives, polyphenols, terpenes and steroids, methylated phenols, and benzopyrans, carbohydrates free fatty acids and triglycerides

The term “alkaloids” as used herein refers to a group of naturally occurring chemical compounds that contain mostly basic nitrogen atoms. In addition to carbon, hydrogen and nitrogen, alkaloids may also contain oxygen, sulfur and more rarely other elements such as chlorine, bromine, and phosphorus. Exemplary alkaloids comprise “True alkaloids”, which contain nitrogen in the heterocycle and originate from amino acids Their characteristic examples are atropine, nicotine, and morphine. This group also includes some alkaloids that besides nitrogen heterocycle contain terpene (e.g., evonine) or peptide fragments (e.g. ergotamine). This group also includes piperidine alkaloids coniine and coniceine although they do not originate from amino acids. “Protoalkaloids”, which contain nitrogen and also originate from amino acids. Examples include mescaline, adrenaline and ephedrine. Polyamine alkaloids—derivatives of putrescine, spermidine, and spermine. Peptide and cyclopeptide alkaloids, Pseudalkaloids—alkaloid-like compounds that do not originate from amino acids. This group includes, terpene-like and steroid-like alkaloids, as well as purine-like alkaloids such as caffeine, theobromine, theacrine and theophylline. Alkaloids are produced by a large variety of organisms, including bacteria, fungi, plants, and animals, and are part of the group of natural products (also called secondary metabolites). Examples of alkaloids can be the local anesthetic and stimulant cocaine, the psychedelic psilocin, the stimulant caffeine, nicotine, the analgesic morphine, the antibacterial berberine, the anticancer compound vincristine, the antihypertension agent reserpine, the cholinomimeric galantamine, the spasmolysis agent atropine, the vasodilator vincamine, the anti-arrhythmia compound quinidine, the anti-asthma therapeutic ephedrine, and the antimalarial drug quinine.

The term “phenolic acids and derivatives” as used herein refers to organic compounds containing a carboxylic acid function and a phenolic ring, like benzoic and cinnamic acids derivatives. They can be found in many species of plants as in Camellia sinensis, Ribes nigrum, Vaccinium myrtillus and have some pharmacological properties in vitro. Recent interest in phenolic acids and derivatives comes from their potential protective role, through ingestion of fruits and vegetables, against oxidative damage diseases. They form a diverse group that includes the widely distributed benzoic acids derivatives (Gallic acid, Vanillic acid) and cinnamic acid derivatives (Chlorogenic acid or 3-dicafeoylquinique) found in the Cynarus scolymus. Many plant phenolic compounds are polymerised into larger molecules as Catechins, tannins and Procyanidins. Phenolic acids and derivatives can include cinnamic aldehyde, trans cinnamic acid, 3,4-dihydroxy-hydrocinnamic acid, chlorogenic acid or a combination thereof.

The term “polyphenols”, also known as polyhydroxyphenols, as used herein refers to structural class of organic chemicals characterized by the presence of large multiples of phenol structural units. The number and characteristics of these phenol structures underlie the unique physical, chemical, and biological (metabolic, toxic, therapeutic, and addi) properties of particular members of the class. Examples include tannic acid (image at right), and ellagitannin (image below). The historically important chemical class of tannins is a subset of the polyphenols. Polyphenols can include one or more substance selected from the group including flavonoids, lignans or a combination thereof.

The term “terpenes” as used herein refers to a large and diverse class of organic compounds, produced by a variety of plants. They are often strong-smelling, and thus may protect the plants that produce them by deterring parasites. Many terpenes are aromatic hydrocarbons and thus may have had a protective function. The difference between terpenes and terpenoids is that terpenes are hydrocarbons, whereas terpenoids contain additional functional groups. Terpenes are the major components of resin, and of turpentine produced from resin. In addition to their roles as end-products in many organisms, terpenes are major biosynthetic building blocks within nearly every living creature. When terpenes are modified chemically, such as by oxidation or rearrangement of the carbon skeleton, the resulting compounds are generally referred to as terpenoids. Some authors will use the term terpene to include all terpenoids. Terpenoids are also known as isoprenoids.

Terpenes and terpenoids are the primary constituents of the essential oils of many types of plants and flowers. Synthetic variations and derivatives of natural terpenes and terpenoids also greatly expand the variety of aromas used in perfumery and flavors used in food additives. Vitamin A is a terpene. Terpenes are released by trees more actively in warmer weather, acting as a natural form of cloud seeding. The clouds reflect sunlight, allowing the forest to regulate its temperature. The aroma and flavor of hops, highly desirable in some beers, comes from terpenes. Of the terpenes in hops myrcene, β-pinene, β-caryophyllene, and α-humulene are found in the largest quantities.

Terpenes are derived biosynthetically from units of isoprene, which has the molecular formula C5H8. The basic molecular formulae of terpenes are multiples of that, (C₅H₈)n where n is the number of linked isoprene units. This is called the isoprene rule or the C₅ rule. The isoprene units can be linked together “head to tail” to form linear chains or they can be arranged to form rings. One can consider the isoprene unit as one of nature's common building blocks. Isoprene itself does not undergo the building process, but rather activated forms, isopentenyl pyrophosphate (IPP or also isopentenyl diphosphate) and dimethylallyl pyrophosphate (DMAPP or also dimethylallyl diphosphate), are the components in the biosynthetic pathway. IPP is formed from acetyl-CoA via the intermediacy of mevalonic acid in the HMG-CoA reductase pathway.

The term “steroids” as used herein intends organic compounds that contain a characteristic arrangement of four cycloalkane rings that are joined to each other. Examples of steroids include the dietary lipid cholesterol, the sex hormones estradiol and testosterone and the anti-inflammatory drug dexamethasone. The core of steroids is composed of seventeen carbon atoms bonded together that take the form of four fused rings: three cyclohexane rings (designated as rings A, B and C in the scheme below) and one cyclopentane ring (the D ring).

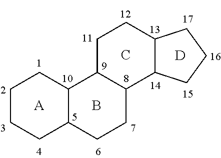

The steroids vary by the functional groups attached to this four-ring core and by the oxidation state of the rings. Sterols are special forms of steroids, with a hydroxyl group at position-3 and a skeleton derived from cholestane. Hundreds of distinct steroids are found in plants. All steroids are made in cells either from the sterols lanosterol (animals and fungi, see below right) or from cycloartenol (plants). Both lanosterol and cycloartenol are derived from the cyclization of the triterpene squalene. In addition, steroids are a class of organic compounds with a chemical structure that contains the core of gonane or a skeleton derived therefrom. Usually, methyl groups are present at the carbons C-10 and C-13—an alkyl side-chain at carbon C-17 may also be present. Gonane is the simplest possible steroid and is composed of seventeen carbon atoms, bonded together to form four fused rings. The three cyclohexane rings (designated as rings A, B, and C in the figure below) form the skeleton of phenanthrene; ring D has a cyclopentane structure. Hence, together they are called cyclopentaphenanthrene. Commonly, steroids have a methyl group at the carbons C-10 and C-13 and an alkyl side chain at carbon C-17. Further, they vary by the configuration of the side chain, the number of additional methyl groups, and the functional groups attached to the rings. For example, sterols have a hydroxyl group attached at position C-3. Steroids can include one or more substance selected from the group including limonene, α-copaene, β-sitosterol or a combination thereof.

The term “methylated phenols”, also called methylphenol, as used herein intends aromatic compounds derived from phenol, existing in three isomeric forms: found in coal tar and creosote and used in making synthetic resins and as an antiseptic and disinfectant; hydroxytoluene. The formula of methylated phenols is C₆H₄(CH₃)OH. Methylated phenols can include tocopherols.

The term “benzopyrans” as used herein, and unless otherwise specified, refers to polycyclic organic compounds that results from the fusion of a benzene ring to a heterocyclic pyran ring. According to IUPAC nomenclature it is called chromene. There are two isomers of benzopyran that vary by the orientation of the fusion of the two rings compared to the oxygen, resulting in 1-benzopyran (chromene) and 2-benzopyran (isochromene)—the number denotes where the oxygen atom is located by standard naphthalene-like nomenclature. Commonly, benzopyran is encountered in the reduced state, in which it is partially saturated with one hydrogen atom, introducing a tetrahedral CH2 group in the pyran ring. Therefore, there are many structural isomers owing to the multiple possible positions of the oxygen atom and the tetrahedral carbon atom. Benzopyrans can include coumarin.

The term “free fatty acids”, as used herein, refers to carboxylic acids with a long aliphatic tail (chain), which is either saturated or unsaturated. Most naturally occurring fatty acids have a chain of an even number of carbon atoms, from 4 to 28. Fatty acids are usually derived from triglycerides or phospholipids. When they are not attached to other molecules, they are known as “free” fatty acids. Fatty acids that have carbon-carbon double bonds are known as unsaturated. Fatty acids without double bonds are known as saturated. They differ in length as well. Short-chain fatty acids (SCFA) are fatty acids with aliphatic tails of fewer than six carbons (i.e. butyric acid). Medium-chain fatty acids (MCFA) are fatty acids with aliphatic tails of 6-12 carbons, which can form medium-chain triglycerides. Long-chain fatty acids (LCFA) are fatty acids with aliphatic tails 13 to 21 carbons. Very long chain fatty acids (VLCFA) are fatty acids with aliphatic tails longer than 22 carbons. Unsaturated fatty acids have one or more double bonds between carbon atoms. (Pairs of carbon atoms connected by double bonds can be saturated by adding hydrogen atoms to them, converting the double bonds to single bonds. Therefore, the double bonds are called unsaturated.) Examples of Unsaturated Fatty Acids are Myristoleic acid CH3(CH2)3CH═CH(CH2)7COOH; palmitoleic acid; Sapienic acid CH3(CH2)8CH═CH(CH2)4COOH; Oleic acid CH3(CH2)7CH═CH(CH2)7COOH; Elaidic acid CH3(CH2)7CH═CH(CH2)7COOH; Vaccenic acid CH3(CH2)5CH═CH(CH2)9COOH; Linoleic acid CH3(CH2)4CH═CHCH2CH═CH(CH2)7COOH; Linoelaidic acid CH3(CH2)4CH═CHCH2CH═CH(CH2)7COOH; α-Linolenic acid CH3CH2CH═CHCH2CH═CHCH2CH═CH(CH2)7COOH; Arachidonic acid CH3 (CH2)4CH═CHCH2CH═CHCH2CH═CHCH2CH═CH(CH2)3COOH; Eicosapentaenoic acid CH3CH2CH═CHCH2CH═CHCH2CH═CHCH2CH═CHCH2CH═CH(CH2)3COOH. Examples of Saturated Fatty Acids are Caprylic acid CH3(CH2)6COOH; Capric acid CH3(CH2)8COOH; Lauric acid CH3(CH2)10COOH; Myristic acid CH3(CH2)12COOH; Palmitic acid CH3(CH2)14COOH; Stearic acid CH3 (CH2)16COOH; Arachidic acid CH3 (CH2)18COOH.

The term “triglycerides” as used therein relates to esters derived from glycerol and three fatty acids. As a blood lipid, they help enable the bidirectional transference of adipose fat and blood glucose from the liver. There are many triglycerides: depending on the oil source, some are highly unsaturated, some less so. Saturated compounds are “saturated” with hydrogen—all available places where hydrogen atoms could be bonded to carbon atoms are occupied. Unsaturated compounds have double bonds (C═C) between carbon atoms, reducing the number of places where hydrogen atoms can bond to carbon atoms. Saturated compounds have single bonds (C—C) between the carbon atoms, and the other bond is bound to hydrogen atoms (for example ═CH—CH═, —CH2-CH2-, etc.). Unsaturated fats have a lower melting point and are more likely to be liquid. Saturated fats have a higher melting point and are more likely to be solid at room temperature. Triglycerides are the main constituents of vegetable oil (typically more unsaturated) and animal fats (typically more saturated. Triglycerides are formed by combining glycerol with three molecules of fatty acid. Alcohols have a hydroxyl (HO—) group. Organic acids have a carboxyl (—COOH) group. Alcohols and organic acids join to form esters. The glycerol molecule has three hydroxyl (HO—) groups. Each fatty acid has a carboxyl group (—COOH). In triglycerides, the hydroxyl groups of the glycerol join the carboxyl groups of the fatty acid to form ester bonds: HOCH2CH(OH)CH2OH+RCO2H+R′CO2H+R″CO2H→RCO2CH2CH(O2CR′)CH2CO2R″+3H2O. The three fatty acids (RCO2H, R′CO2H, R″CO2H in the above equation) are usually different, but many kinds of triglycerides are known. The chain lengths of the fatty acids in naturally occurring triglycerides vary, but most contain 16, 18, or 20 carbon atoms. Natural fatty acids found in plants and animals are typically composed of only even numbers of carbon atoms, reflecting the pathway for their biosynthesis from the two-carbon building-block acetyl CoA. Bacteria, however, possess the ability to synthesise odd- and branched-chain fatty acids. As a result, ruminant animal fat contains odd-numbered fatty acids, such as 15, due to the action of bacteria in the rumen. Many fatty acids are unsaturated, some are polyunsaturated, e.g., those derived from linoleic acid. Most natural fats contain a complex mixture of individual triglycerides.

The term “carbohydrates” as used therein relates to large biological molecules, or macromolecules, consisting of carbon (C), hydrogen (H), and oxygen (O) atoms, usually with a hydrogen:oxygen atom ratio of 2:1 (as in water); in other words, with the empirical formula Cm(H2O)n (where m could be different from n). The term is most common in biochemistry, where it is a synonym of saccharide. The carbohydrates (saccharides) are divided into four chemical groups: monosaccharides, disaccharides, oligosaccharides, and polysaccharides. In general, the monosaccharides and disaccharides, which are smaller (lower molecular weight) carbohydrates, are commonly referred to as sugars. More in particular, natural saccharides are generally built of simple carbohydrates called monosaccharides with general formula (CH2O)n where n is three or more. A typical monosaccharide has the structure H—(CHOH)x(C═O)—(CHOH)y-H, that is, an aldehyde or ketone with many hydroxyl groups added, usually one on each carbon atom that is not part of the aldehyde or ketone functional group. Examples of monosaccharides are glucose, fructose, and glyceraldehydes. However, some biological substances commonly called “monosaccharides” do not conform to this formula (e.g., uronic acids and deoxy-sugars such as fucose), and there are many chemicals that do conform to this formula but are not considered to be monosaccharides (e.g., formaldehyde CH2O and inositol (CH2O)6). Monosaccharides can be linked together into what are called polysaccharides (or oligosaccharides) in a large variety of ways.

In some embodiments, the extraction can be performed by contacting aerial parts of the plant belonging to the jasminum genus and the plant belonging to the cinnamomum genus in ethanol for a time and under condition to obtain an ethanolic extract of the plant belonging to the jasminum genus and the plant belonging to the cinnamomum genus, so that the extract comprises alkaloids, phenolic acids and derivatives, polyphenols, terpenes and steroids, methylated phenols, and benzopyrans, carbohydrates free fatty acids and triglycerides (herein also two plant extract)

In embodiments, herein described the plant belonging to the genus of Jasminum is in a flowering stage. More in particular, an aerial part of the plant belonging to Jasminum genus, such as leaves, branches and flowers of the Jasmine in a flowering stage can be put in ethanol. In relation to the plant belonging to the genus Cinnamomum, such plant can be used in the form of sticks, such as for example cinnamon sticks. More in particular, in some embodiments, the cinnamon sticks can be put in ethanol.

In some embodiments, the ethanolic plant extract is obtained by contacting aerial parts of at least a first plant and a second plant with ethanol with a volume/volume ratio first plant:ethanol from about 1:1.25 to about 1:1.75 and a volume/volume ratio second plant:ethanol from about 1:10 to about 1:18 wherein the first plant belongs to plant genus Jasminum and is in flowering stage, and the second plant belongs to plant genus Cinnamomum. In some embodiments the analogy in volumes is first plant: ethanol 95 is about 1:1.5. In some embodiments the analogy in volumes is second plant: ethanol 95 is about 1:14.

In some embodiments, the contacting is performed by submerging at least part of the aerial parts of plant belonging to the jasminum genus and the plant belonging to the cinnamomum genus in ethanol at a temperature comprised between 15° C. and 35° C. for a time comprised between 7 days and 21 days, depending on the temperature selected and the ethanol solvent selected. In some embodiments, the contacting is performed at room temperature for 14 days using ethanol 95 (see Example 1)

In some embodiments, the plant and/or any plant-related products can be put in ethanol and left in ethanol for greater or lower time period, such as some days or some weeks depending on the temperature of extractions and ethanol solvents selected as will be understood by a skilled person.

After extraction, the ethanolic solution can be removed from the plant parts, e.g. the ethanolic solution can be drained to remove all or part of the rest of plant and/or plant-related products.

In some embodiments, ethanolic plant extracts herein described comprise indole alkaloids, phenolic acids and derivatives (mainly cinnamic aldehyde, trans cinnamic acid, 3,4-dihydroxy-hydrocinnamic acid), polyphenols (mainly flavonoids and lignans), carbohydrates (mainly sucrose and glucose), terpenes and steroids (such as limonene, α-copaene, β-sitosterol), and benzopyrans (mainly coumarin), as well as free fatty acids and triglycerides

In some embodiments, the aerial parts of at least a first and a second plants further comprise aerial parts of a third plant, and the contacting further comprises contacting the aerial parts of the third plants with ethanol In those embodiments, the third plant belongs to the genus Coffea and the aerial parts are green coffee beans.

The term “Coffea” as used herein indicates a genus of flowering plants whose seeds, called coffee beans, are used to make coffee. It is a member of the Rubiaceae family. They are shrubs or small trees native to tropical and southern Africa and tropical Asia. Exemplary species of the Coffea genus comprise Coffea abbayesii, Coffea affinis, Coffea alleizettii, Coffea ambanjensis, Coffea ambongenis, Coffea andrambovatensis, Coffea ankaranensis, Coffea anthonyi, Coffea arabica L., Coffea arenesiana, Coffea augagneurii, Coffea bakossii, Coffea benghalensis, Coffea bertrandii, Coffea betamponensis, Coffea bissetiae, Coffea boinensis, Coffea boiviniana A. P. Davis & Rakotonas, Coffea bonnieri, Coffea brassii, Coffea brevipes, Coffea bridsoniae, Coffea buxifolia, Coffea canephora, Coffea carrissoi, Coffea charrieriana, Coffea cochinchinensis, Coffea commersoniana, Coffea congensis, Coffea costatifructa Bridson, Coffea coursiana J.-F. Leroy, Coffea dactylifera, Coffea, Coffea dubardii Jum, Coffea ebracteolata, Coffea eugenioides, Coffea fadenii, Coffea farafanganensis, Coffea floresiana, Coffea fotsoana, Coffea fragilis, Coffea fragrans, Coffea gallienii Dubard, Coffea grevei, Coffea heimii, Coffea×heterocalyx, Coffea homollei, Coffea horsfieldiana, Coffea humbertii, Coffea humblotiana, Coffea humilis, Coffea jumellei, Coffea kapakata, Coffea kianjavatensis, Coffea kihansiensis, Coffea kimbozensis, Coffea kivuensis, Coffea labatii, Coffea lancifolia, Coffea lebruniana, Coffea leonimontana, Coffea leroyi A. P. Davis, Coffea liaudii J.-F. Leroy ex A. P. Davis, Coffea liberica, Coffea ligustroides, Coffea littoralis, Coffea lulandoensis, Coffea mabesae, Coffea macrocarpa, Coffea madurensis, Coffea magnistipul, Coffea malabarica, Coffea mangoroensis, Coffea manombensis, Coffea mapiana, Coffea mauritiana, Coffea mayombensis, Coffea mcphersonii, Coffea melanocarpa, Coffea merguensis., Coffea millotii, Coffea minutiflora, Coffea mogenetii, Coffea mongensis, Coffea montekupensis, Coffea montis-sacri, Coffea moratii, Coffea mufindiensis, Coffea myrtifolia, Coffea namorokensis, Coffea neobridsoniae, Coffea neoleroya, Coffea perrieri, Coffea pervilleana, Coffea pocsii, Coffea pseudozanguebariae, Coffea pterocarpa, Coffea racemosa Lour., Coffea rakotonasoloi, Coffea ratsimamangae, Coffea resinosa, Coffea rhamnifolia, Coffea richardii, Coffea sahafaryensis, Coffea sakarahae, Coffea salvatrix, Coffea sambavensis, Coffea sapinii, Coffea schliebenii, Coffea semsei, Coffea sessiliflora, Coffea stenophylla, Coffea tetragona, Coffea togoensis, Coffea toshii, Coffea travancorensis, Coffea tricalysioides, Coffea tsirananae, Coffea vatovavyensis, Coffea vavateninensis, Coffea vianneyi, Coffea vohemarensis, Coffea wightiana, Coffea zanguebariae.

The term “green coffee bean” as used herein relates to a unroasted seed of the a plant belonging to the coffea genus, and is the source for coffee. It is the pit inside the red or purple fruit often referred to as a cherry. Even though they are seeds, they are incorrectly referred to as ‘beans’ because of their resemblance to true beans. Coffee seeds consist mostly of endosperm. The green coffee bean can be of any varieties of coffee plant, such as for example Arabica and the Robusta.

In embodiments, herein described ethanol extraction performed with ethanol selected between ethanol 90% to ethanol 99% and with aerial parts of the aerial parts of plant belonging to the jasminum genus and the plant belonging to the cinnamomum genus and green beans from the plant belonging to coffea genus provides a mixture comprising alkaloids, phenolic acids and derivatives, polyphenols, terpenes and steroids, methylated phenols, and benzopyrans, carbohydrates free fatty acids and triglycerides, which is enriched in alkaloids (mainly caffeine), phenolic acids (mainly chlorogenic acid) and methylated phenols (mainly tocopherols).

In some embodiments, the alkaloids include caffeine. In some embodiments, the phenolic acids and derivatives include one or more substance selected from the group consisting of cinnamic aldehyde, trans cinnamic acid, 3,4-dihydroxy-hydrocinnamic acid, chlorogenic acid or a combination thereof. In some embodiments, the polyphenols includes one or more substance selected from the group including flavonoids, lignans or a combination thereof. In some embodiments, the carbohydrates include one or more substance selected from the group including sucrose, glucose or a combination thereof. In some embodiments, the steroids include one or more substance selected from the group including limonene, α-copaene, β-sitosterol or a combination thereof. In some embodiments, the methylated phenols include tocopherols. In some embodiments, the benzopyrans include coumarin.

In embodiments, wherein the extraction is performed on three plants the contacting can be performed by submerging at least part of the aerial parts of plant belonging to the jasminum genus and the plant belonging to the cinnamomum genus and the plant belonging to coffea genus in ethanol at a temperature comprised between 15° C. and 35° C. for a time comprised between 7 days and 21 days, depending on the temperature selected and the ethanol solvent selected. In some embodiments, the contacting is performed at room temperature for 14 days using ethanol 95 (see Example 1)

In some embodiments, when the plant extract includes the three plant extracts, wherein green coffee beans are added to ethanol such that the analogy in volume is green coffee beans:ethanol from about 1:25 to about 1:35. In one embodiment the analogy in volume is green coffee beans: ethanol is 1:30. The extraction, such as the extraction of leaves, branches and flowers of Jasmine and of cinnamon sticks and green coffee beans, can be performed at a temperature comprised from about 15° C. and about 35° C. at room temperature for from 11 to 17 days. In one embodiments, the extraction is performed for 14 days.

In some embodiments, the plant and/or any plant-related products can be put in ethanol and left in ethanol for greater or lower time period, such as some days or some weeks depending on the temperature of extractions and ethanol solvents selected.

After extraction, the ethanolic solution can be drained to remove all or part of the rest of plant and/or plant-related products.

According to further aspects of the present disclosure, the plant extract having an effect on biological activity in an individual includes a combination of alkaloids, phenolic acids and derivatives, polyphenols, terpenes and steroids, methylated phenols, and benzopyrans, carbohydrates, free fatty acids and triglycerides. In particular, in relation to such components, it has been demonstrated a plant extract having an effect on biological activity in an individual include a combination of the following components: alkaloids, phenolic acids and derivatives, polyphenols, terpenes and steroids, methylated phenols, and benzopyrans, free fatty acids and triglycerides. Depending on the collection time of the plant tissues the quantitative, as well as the qualitative chemical composition can exhibit substantial variation of such components.

In some embodiments, the plant belonging to the genus Jasminum is jasmine. In some embodiments, the plant belonging to the genus Cinnamomum is cinnamon. In some embodiments, the plant belonging to the genus coffea are Coffea Arabica and/or Coffea Robusta.

In some embodiments, one or more ethanolic plant extracts according to the present disclosure can be used in combination with one or more active agents to provide formulations having a biological activity.

The term “active agent” or “active ingredient” as used herein indicates a substance that have an effect on a target system. An exemplary target system comprise a biological environment which refers to any biological setting, including, for example, ecosystems, orders, families, genera, species, subspecies, organisms, tissues, cells, viruses, organelles, cellular substructures, prions, and samples of biological origin. Exemplary active agents or ingredient are sugars, amino acids, peptides, proteins, oligonucleotides, polynucleotides, polypeptides, organic molecules, haptens, epitopes, biological cells, parts of biological cells, vitamins, hormones and the like. Additional active agent can be pharmacologically active agent which have a direct effect in the diagnosis, cure, mitigation, treatment or prevention of disease, or to have direct effect in restoring, correcting or modifying physiological functions in individuals.

As used herein the term “formulation” indicates is a mixture or a structure such as a capsule, a pill, tablet, or an emulsion, prepared according to a specific procedure (called a “formula”). Formulations are an aspect of creating medicines, directed to ensure that the active part of the drug is delivered to the correct part of the body, in the right concentration, and at the right rate (not too fast and not too slowly). A good example are drug delivery systems that exploit supersaturation. Typically formulations can include ingredients to provide an acceptable taste (in the case of pills, tablets or syrups or other formulations for oral administration), in general last long enough in storage still to be safe and effective when used, and be sufficiently stable both physically and chemically to be transported from where they are manufactured to the eventual consumer. Competently designed formulations for particular applications are safer, more effective, and more economical than any of their components used singly. Exemplary formulations are commercially produced for drugs, cosmetics, coatings, dyes, foods, and many others.

The term “biological activity” as used herein refers to the ability to affect one or more of the interrelated physical situations, reactions and equilibriums that define the physical status of the body of an individual, as a whole or of one or more of its parts. Biological activities herein described comprise the ability of a substance to affect states of the living individual that is associated to a functional normality or abnormality of the body or of any of its parts, and functioning of the body or of any of its parts and is typically manifested by distinguishing signs and symptoms. A change or an effect of a biological activity can be the consequence of beneficial or adverse effects of one or more substance on living matter, or on an individual.

Accordingly, in some embodiments, formulations having an effect on biological activity in an individual comprising one or more ethanolic extract here described have one effect on any cell tissue in an individual, such as a human or animal body

In particular in some embodiments, one or more ethanolic plant extracts herein described can be combined with one or more active agents to provide formulations that can be used to affect expression and activity of a protein or other compound that contributes to the cause of a specific biochemical reaction or bodily process (e.g. transcription factors and signaling molecules) in an individual

The term “individual” or “subject” or “patient” as used herein in the context of treatment includes a single animal and in particular higher animals and in particular vertebrates such as mammals and in particular human beings, having an immune system, a vascular system and a nervous system, and producing factors such as the human NGF, NF-kB, TRAF, immunoglobulins, melanin, Tau protein, Htr1b or corresponding factors as will be understood by a skilled person.

In some embodiments, a formulation of the disclosure comprise one or more ethanolic plant extract herein described together with one substance selected from the group consisting of avocado oil; copper sulfate, ester-vitC; Para aminobenzoic acid; Cod liver oil; Vitamin A; Vitamin D; EPA and DHA and/or a combination thereof.

In some embodiments, a formulation can comprise the plant extract of the first plant belonging to the Jasminum genus and the second plant belonging to the Cinnamonum genus (herein also two-plants extract). In those embodiments, the formulation can comprise from about 0, 25 ml to about 0, 05 ml of the two-plant ethanolic plant extract.

In some embodiments, a formulation can comprise the plant extract of the first plant belonging to the Jasminum genus, the second plant belonging to the Cinnamonum genus and the third plant belonging to the Coffea genus (herein also three-plants extract). In those embodiments, the formulation can comprise from about 0, 25 ml to about 0, 05 ml of the two-plant ethanolic plant extract.

In some embodiments, a formulation including the plant extract of the two-plants (a first plant and a second plant). More in particular, the formulation can comprise one or more ethanolic plant extract (e.g. 3 drops or 0.15 ml) together with 0.1-0.025 ml of avocado oil, 1.2-0.4 mg of copper sulfate, 400-100 mg of ester vitC, 400-100 mg of Para amino benzoic acid, 400-100 mg of Cod liver oil, 600-200 gRE of vitA, 2-0.5 μg of vitD, 30-10 mg of EPA and/or 30-10 mg of DHA.

In some embodiments, the formulation can include 0.15 ml±10%% of the ethanolic plant extract (two-plant extract and/or three plant extract), 0.05 ml±10% of avocado oil, 0.83 mg±10% of copper sulfate, 250 mg±10% of ester vitC, 275 mg±10% of Para amino benzoic acid, 250 mg±10% of Cod liver oil, 400gRE±10% of vitA, 1.25 μg±10% of vitD, 22 mg±10% of EPA and/or 19 mg±10% of DHA. In some of those embodiments, administering of the formulation to an individual can result in activation of the-NFkb factor up to 10 times with respect to a base level that can be measured before administration

In some embodiments, a formulation can include the plant extract of the three-plants (a first plant, a second plant and a third plant). More in particular, the formulation can include 0, 25-0, 05 ml of the three ethanolic plant extract, 0.1-0.025 ml of avocado oil, 1.2-0.4 mg of copper sulfate, 400-100 mg of ester vitC, 400-100 mg of Para amino benzoic acid, 400-100 mg of Cod liver oil, 600-200 gRE of vitA, 2-0.5 μg of vitD, 30-10 mg of EPA and 30-10 mg of DHA.

In some embodiments, it is provided a formulation including the plant extract of the three-plants. More in particular, the formulation can include 0.15 ml±10% or 3 drops±10% of the ethanolic plant extract, 0.05 ml±10% of avocado oil, 0.83 mg±10% of copper sulfate, 250 mg±10% of ester vitC, 275 mg±10% of Para amino benzoic acid, 250 mg±10% of Cod liver oil, 400gRE±10% of vitA, 1, 25 μg±10% of vitD, 22 mg±10% of EPA and 19 mg±10% of DHA. In some of those embodiments, administering of the formulation to an individual can result in activation of NFkb factor up to 10 times with respect to a base level that can be measured before administration.

In some embodiments, the formulation further comprises excipients and in particular pharmaceutically acceptable excipients. The term “excipients” indicates any of various media acting usually as solvents, carriers, binders or diluents for the rifaximin comprised in a composition as an active ingredient. Exemplary pharmaceutically acceptable excipients in the sense of the present disclosure comprise, lubricants, glidants, diluents, buffering agents, opacifiers, plasticizers, colouring agents, agents capable of providing a controlled release.

In some embodiments, an ethanolic plant extract herein in combination with one or more active agent can show a biological activity comprising at least one of the following biological activities activating transcription factor NF-kB; blocking binding of protein TRAF6 to signal transduction pathway that begins from the permanently activated CD40 in epithelial cells and B-lymphocytes mimics pathological autoimmune diseases; eliminating the presence of autoantibodies against DNA; eliminating deposition of immunoglobulin IgG; eliminating perivascular liver inflammation; increasing the biological activity and reactivity of the metabolism of tyrosine; increasing the biological activity and reactivity of melanogenesis; increasing the biological activity of serotonergic synapses groups; increasing the expression of NGF (Nerve Growth Factor); increasing the biological activity of cholinergic synapses; decreasing the phosphorylation of Tau protein; reducing gene expression of Htr1b gene (receptor 5-hydroxytryptamine or serotonin receptor 1B).

In particular in some embodiments, a formulation including the two-plant extracts (herein also agent X) together with avocado oil; copper sulfate, ester-vitC; Para aminobenzoic acid; Cod liver oil; Vitamin A; Vitamin D; EPA and DHA have been found to be able elicit all the effects mentioned in the preceding paragraph (herein also Factor X) (see Examples 14-22)

In particular, in some embodiments, formulation herein described as Factor X can comprise 0, 25-0, 05 ml of the two-plant ethanolic plant extract, 0.1-0.025 ml of avocado oil, 1.2-0.4 mg of copper sulfate, 400-100 mg of ester vitC, 400-100 mg of Para amino benzoic acid, 400-100 mg of Cod liver oil, 600-200 gRE of vitA, 2-0.5 μg of vitD, 30-10 mg of EPA and 30-10 mg of DHA.

In some of those embodiments, the formulation Factor X can include 0.15 ml±10% of the two-plant ethanolic plant extract, 0.05 ml±10% of avocado oil, 0.83 mg±10% of copper sulfate, 250 mg±10% of ester vitC, 275 mg±10% of Para amino benzoic acid, 250 mg±10% of Cod liver oil, 400gRE±10% of vitA, 1, 25 μg±10% of vitD, 22 mg±10% of EPA and 19 mg±10% of DHA.

In some embodiments, formulations including the three plant extract (herein also Agent Y) together with avocado oil; copper sulfate, ester-vitC; Para aminobenzoic acid; Cod liver oil; Vitamin A; Vitamin D; EPA and DHA have been found to be able elicit all the effects mentioned in the preceding paragraph (herein also Factor Y) (see Examples 23-38)

In some embodiments, formulation herein described as Factor Y can comprise 0, 25-0, 05 ml of the ethanolic plant extract, 0.1-0.025 ml of avocado oil, 1.2-0.4 mg of copper sulfate, 400-100 mg of ester vitC, 400-100 mg of Para amino benzoic acid, 400-100 mg of Cod liver oil, 600-200 gRE of vitA, 2-0.5 μg of vitD, 30-10 mg of EPA and 30-10 mg of DHA.

In some embodiments, the formulation Facto Y can include 0.15 ml±10% of the ethanolic plant extract, 0.05 ml±10% of avocado oil, 0.83 mg±10% of copper sulfate, 250 mg±10% of ester vitC, 275 mg±10% of Para amino benzoic acid, 250 mg±10% of Cod liver oil, 400gRE±10% of vitA, 1, 25 μg±10% of vitD, 22 mg±10% of EPA and 19 mg±10% of DHA.

The doses reported here above are considered as a human dose. In embodiments, wherein the individual is an animal, or the method is performed in vitro, adjustments to identify the correct dose can be performed by a skilled person upon reading of the disclosure. For example in mice, an amount 1:10 of the human dose can be administered every 12 hours. In embodiments, wherein administration is performed in vitro (e.g. cell cultures) an amount 1:100 of the human dose can be used.

In some embodiments, an ethanolic plant extract herein in combination with one or more active agent can show a biological activity that indicates ability to use the related formulations in the medical applications. For example one or more of the biological activities selected from activating transcription factor NF-kB; blocking binding of protein TRAF6 to signal transduction pathway that begins from the permanently activated CD40 in epithelial cells and B-lymphocytes mimics pathological autoimmune diseases; eliminating the presence of autoantibodies against DNA; eliminating deposition of immunoglobulin IgG; eliminating perivascular liver inflammation; increasing the biological activity and reactivity of the metabolism of tyrosine; increasing the biological activity and reactivity of melanogenesis; increasing the biological activity of serotonergic synapses groups; increasing the expression of NGF (Nerve Growth Factor); increasing the biological activity of cholinergic synapses; decreasing the phosphorylation of Tau protein; reducing gene expression of Htr1b gene (receptor 5-hydroxytryptamine or serotonin receptor 1B) are known to be associated with various biological states and in particular various conditions in the individual as will be understood by a skilled person.

In particular, in some embodiments herein described formulation comprising one or more ethanolic plant extracts and additional active agent can be used in treatment and/or prevention of a condition in an individual and/or to perform research to identify active agents and formulations that can be used to treat and/or prevent the condition in the individual. The term “treatment” as used herein indicates any activity that is part of a medical care for, or deals with, a condition, medically or surgically. The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a symptom or adverse physiological event in a susceptible individual, as well as modulation and/or amelioration of the status of a clinically symptomatic individual by inhibiting or causing regression of a disorder or disease.

The term “prevention” as used herein with reference to a condition indicates any activity which reduces the burden of mortality or morbidity from the condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “condition” indicates a physical status of the body of an individual (as a whole or as one or more of its parts e.g., body systems), that does not conform to a standard physical status associated with a state of complete physical, mental and social well-being for the individual. Conditions herein described comprise disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms in an individual.

In particular, conditions treatable by formulations according to embodiments herein described comprise conditions treatable by at least one of the following biological activities activating transcription factor NF-kB; blocking binding of protein TRAF6 to signal transduction pathway that begins from the permanently activated CD40 in epithelial cells and B-lymphocytes mimics pathological autoimmune diseases; eliminating the presence of autoantibodies against DNA; eliminating deposition of immunoglobulin IgG; eliminating perivascular liver inflammation; increasing the biological activity and reactivity of the metabolism of tyrosine; increasing the biological activity and reactivity of melanogenesis; increasing the biological activity of serotonergic synapses groups; increasing the expression of NGF (Nerve Growth Factor); increasing the biological activity of cholinergic synapses; decreasing the phosphorylation of Tau protein; reducing gene expression of Htr1b gene (receptor 5-hydroxytryptamine or serotonin receptor 1B).

In some embodiments, a formulation comprising the two plant extract and/or the three plant extracts together with avocado oil; copper sulfate, ester-vitC; Para aminobenzoic acid; Cod liver oil; Vitamin A; Vitamin D; EPA and DHA, can be administered to an individual to provide the individual by oral administration with a daily dosage of 6 drops of the ethanolic plant extract, 2 drops of avocado oil; 1.66 mg±10% of copper sulfate, 500 mg±10% of ester-vitamin C; 550 mg±10% daily para aminobenzoic acid; 500 mg±5% daily of Cod liver oil; 800 μgRE±10% of Vitamin A; 2, 5 μg±10% of Vitamin D; 44 mg±10% of EPA and 39 mg±10% of DHA.

In some embodiments, the above daily dosage is provided by administration 3 drops of Factor X or Factor Y comprising 1 drop of avocado oil; 0.83 mg±10% of copper sulfate, 250 mg±10% of ester-vitamin C; 275±10% daily para aminobenzoic acid; 250 mg±5% of Cod liver oil; 400±10% of Vitamin A; 1, 25 μg±10% of Vitamin D; 22 mg±10% of EPA and 19, 5 mg±10% of DHA performed every 12 hours.

In some embodiments, the effect on biological activity in an individual of the Factor-X and/or Factor Y includes one or more of the following effects: activating transcription factor NF-kB; blocking binding of protein TRAF6 to signal transduction pathway that begins from the permanently activated CD40 in epithelial cells and B-lymphocytes mimics pathological autoimmune diseases; eliminating the presence of autoantibodies against DNA; eliminating deposition of immunoglobulin IgG; eliminating perivascular liver inflammation; increasing the biological activity and reactivity of the metabolism of tyrosine; increasing the biological activity and reactivity of melanogenesis; increasing the biological activity of serotonergic synapses groups; increasing the expression of NGF (Nerve Growth Factor); increasing the biological activity of cholinergic synapses; decreasing the phosphorylation of Tau protein; reducing gene expression of Htr1b gene (receptor 5-hydroxytryptamine or serotonin receptor 1B).

In some embodiments, one or more of the ethanolic plant extract herein described can be used to identify a formulation capable of eliciting a biological response in an individual. The method comprises providing one or more ethanolic plant extracts herein described; adding to the one or more ethanolic plant extracts at least one active agent to provide a candidate formulation; and testing the candidate formulation to detect a biological activity in vitro or in vivo. In some embodiments the at least one active agents comprise at least one substance selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid DHA or a combination thereof and the candidate formulation is a formulation herein described comprising at least additional active agent herein described.

In some embodiments, at least one active agent selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid (DHA) or a combination thereof can be used in a method and use of a biologically active agent to provide a formulation capable of eliciting a biological response in an individual, is described. The method comprises providing the at least one active agent; and adding to the at least one active agent to an ethanolic plant extract to provide a candidate formulation; and testing the candidate formulation to detect a biological activity in vitro or in vivo.

In particular in some embodiments, the adding and testing in the methods to identify a formulation capable of eliciting a biological response in an individual herein described can be performed by processes and techniques identifiable by a skilled person. In particular, the adding can be performed based on methods dependent on the chemical nature and form of the active agent that is added taking into account the physical chemical properties of the ethanolic plant extracts used in the method to provide the candidate formulation as well as the chemical physical properties of other active agents also used in the methods to provide the candidate formulation. In some embodiments the testing can be performed in vivo (e.g. in animals) or in vitro (e.g. on cell cultures).

In some embodiments, the testing can be performed so that a biological activity is detected using methods and techniques identifiable by a skilled person. In some embodiments, the biological activity can be detected by detection of a biomarker in the system investigated.

The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “target” as used herein indicates an analyte of interest. The term “analyte” refers to a substance, compound, moiety, or component whose presence or absence in a sample is to be detected. Analytes include but are not limited to biomolecules and in particular biomarkers. The term “biomolecule” as used herein indicates a substance, compound or component associated with a biological environment including but not limited to sugars, amino acids, peptides, proteins, oligonucleotides, polynucleotides, polypeptides, organic molecules, haptens, epitopes, biological cells, parts of biological cells, vitamins, hormones and the like. The term “biomarker” indicates a biomolecule that is associated with a specific state of a biological environment including but not limited to a phase of cellular cycle, health and disease state. The presence, absence, reduction, upregulation of the biomarker is associated with and is indicative of a particular state.

As disclosed herein, the ethanolic plant extracts and active agents herein described can be provided as a part of systems to perform any of the methods described herein. The systems can be provided in the form of kits of parts. In a kit of parts, the ethanolic plant extracts, active agents and other reagents to perform the methods can be comprised in the kit independently. The ethanolic plant extracts and active agents can be included in one or more compositions, and each of the ethanolic plant extracts and active agents can be in a composition together with a suitable vehicle.

Additional components can include labeled molecules and in particular, labeled polynucleotides, labeled antibodies, labels, microfluidic chip, reference standards, and additional components identifiable by a skilled person upon reading of the present disclosure to identify biomarkers in methods to identify a formulation capable of eliciting a biological response in an individual. The terms “label” and “labeled molecule” as used herein as a component of a complex or molecule referring to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemiluminescence, production of a compound in outcome of an enzymatic reaction and the like.

In some embodiments, detection of a biomarker agent can be carried either via fluorescent based readouts, in which the labeled antibody is labeled with fluorophore, which includes, but not exhaustively, small molecular dyes, protein chromophores, quantum dots, and gold nanoparticles. Additional techniques are identifiable by a skilled person upon reading of the present disclosure and will not be further discussed in detail.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

In some embodiments, the ethanolic plant extracts and active agents herein described can be included in pharmaceutical compositions together with an excipient or diluent. In particular, in some embodiments, disclosed are pharmaceutical compositions which contain at least one multi-ligand capture agent as herein described, in combination with one or more compatible and pharmaceutically acceptable vehicles, and in particular with pharmaceutically acceptable diluents or excipients. In those pharmaceutical compositions the multi-ligand capture agent can be administered as an active ingredient for treatment or prevention of a condition in an individual.

Further characteristics of the present disclosure will become more apparent hereinafter from the following detailed disclosure by way or illustration only with reference to an experimental section.

### EXAMPLES

The plant extracts, formulations, methods and uses herein described are further illustrated in the following examples, which are provided by way of illustration and are not intended to be limiting.

In particular, the following examples illustrate exemplary plant extracts, formulations and uses. A person skilled in the art will appreciate the applicability and the necessary modifications to adapt the features described in detail in the present section, to additional plant extracts, formulations and uses according to embodiments of the present disclosure.

### Example 1

### Preparation of “Two-Plant” Ethanolic Extract (EMM)

The “two-plant” ethanolic extract (EMM) including a combination of ethanolic plant extract of the plant belonging to the genus Cinnamomum and the plant belonging to the genus of Jasminum, hereafter referred to as “two-plant” ethanolic extract, was first prepared as follows.

Leaves, branches and flowers of the Jasmine in a flowering stage and cinnamon sticks were placed in a jar in ethanol and then left at room temperature for 14 days. Ethanol was ethanol 95 and the analogy in volumes is: jasmine:ethanol 95=1:1.5 and cinnamon:ethanol 95=1:14. After the 14 days, the solution was drained and the rest of the plants removed.

### Example 2

### Spectroscopic and Chromatographic Analysis of EMM Extract

GC-MS analysis has been carried out on both fractions EMMA and EMMB identified in outcome of the experiments reported in Example 1.

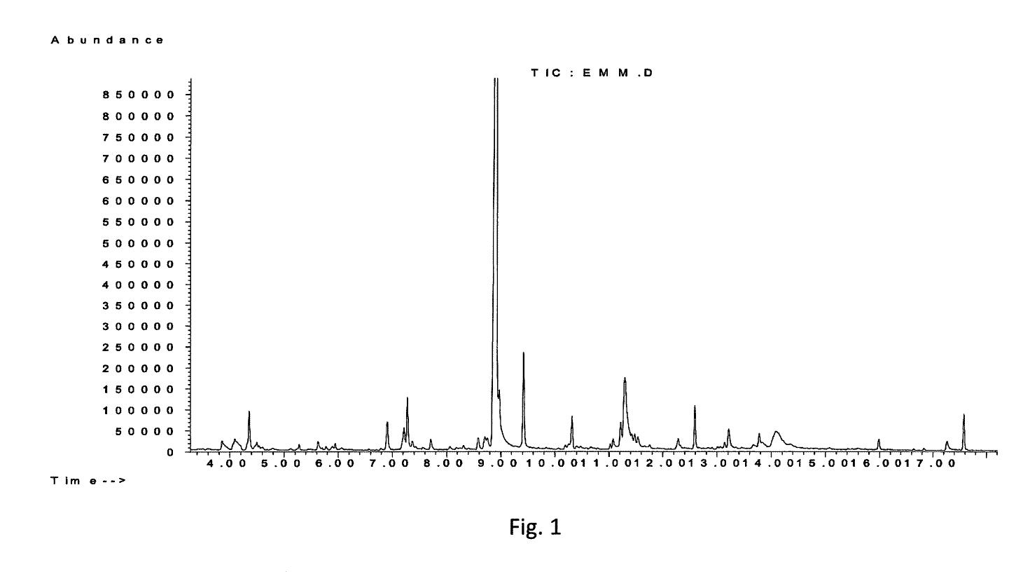

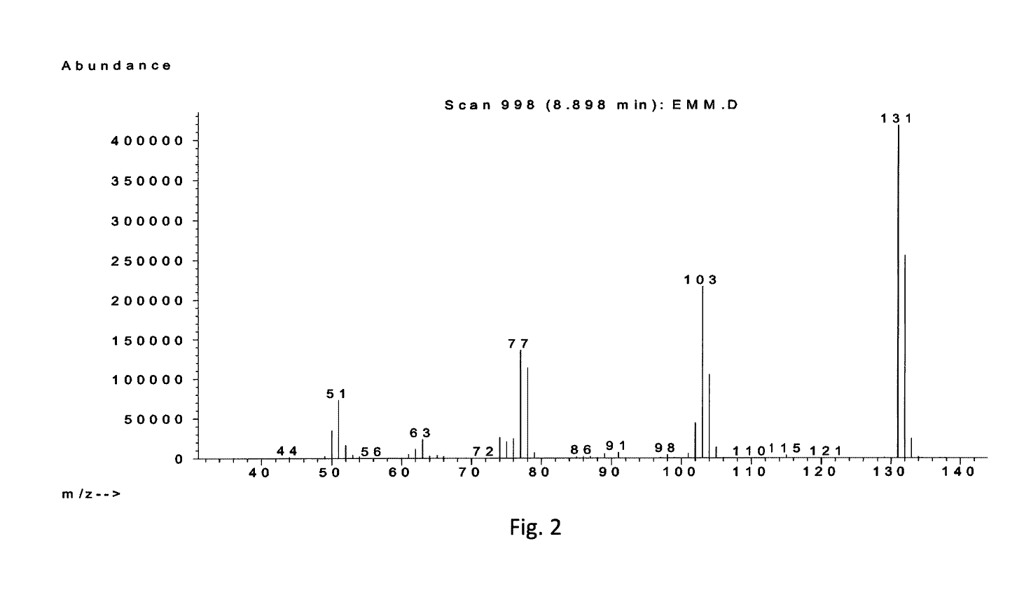

As shown in FIG. 1 and FIG. 2 GC-MS analysis revealed that EMM sample contains benzaldehyde, benzoic acid ethyl ester, α-copaene, cinnamic acetate and coumarin in small proportions, while the major metabolite is cinnamic aldehyde. The compounds were identified by comparing their spectral data (MS) and their retention time with those of standard samples. In particular, FIG. 1 shows GC-MS chromatogram of EMM extract and FIG. 2 shows mass spectrum of major metabolite (cinnamic aldehyde) of EMM extract.

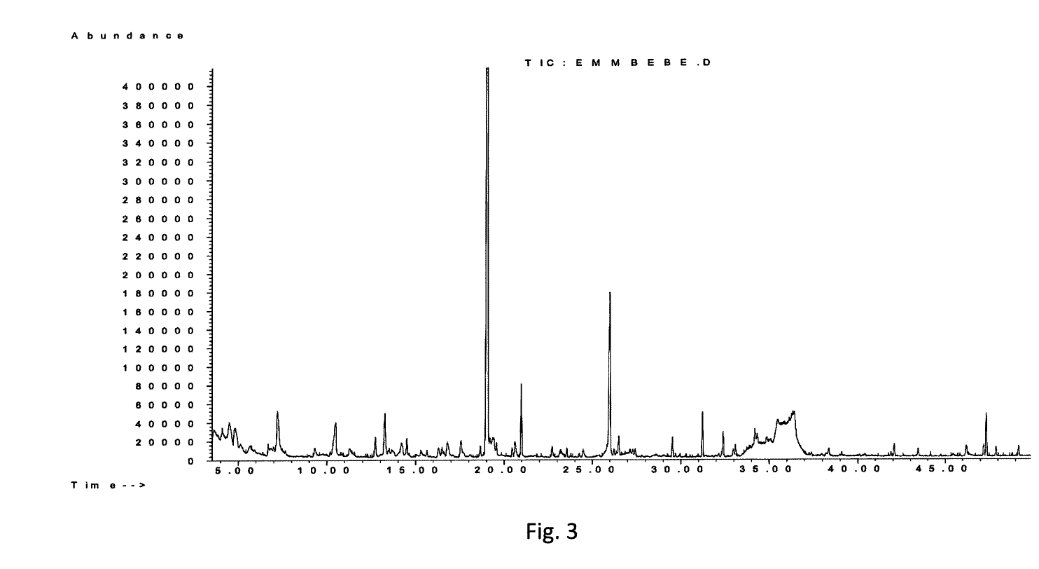

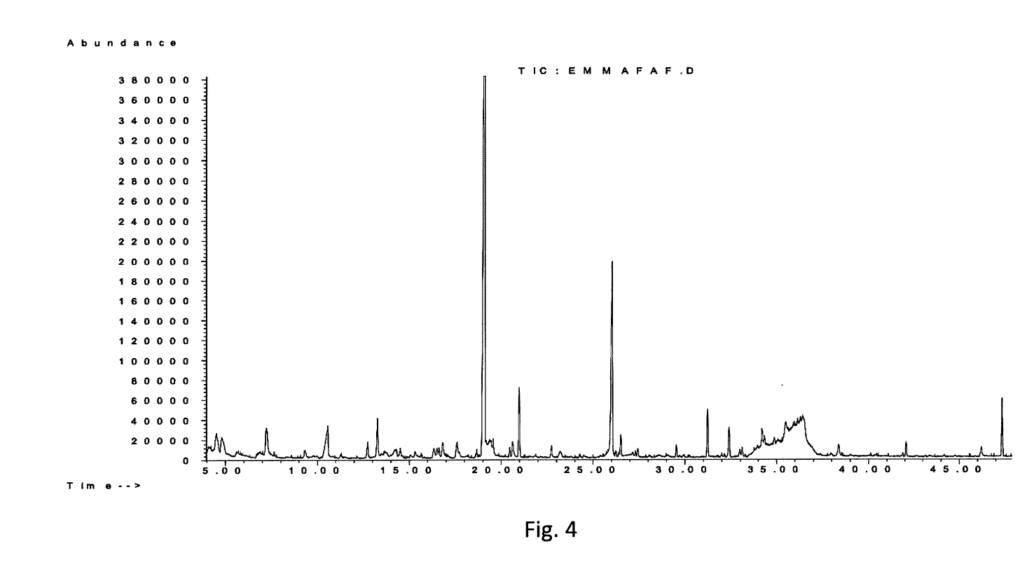

The thermal stability of the initial extract EMM was evaluated by comparing the chemical profile as obtained by GC-MS analysis before and after evaporation of ethanol at elevated temperature. In particular, FIG. 3 shows GC-MS chromatogram of EMM extract before evaporation. FIG. 4 shows GC-MS chromatogram of EMM extract after evaporation FIG. 3 and FIG. 4 proved the compounds' thermal stability.

### Example 3

### Fractionation of EMM Extract in EMMA and EMMB Extracts

The “two-plant” ethanolic extract was further extracted with two-phase liquid system.

The initial extract EMM was subjected to liquid-liquid extraction. 500 ml of EMM (4.46 g diluted in ethanol) were mixed with 200 ml of pentane in separation funnel. Each solvent phase was collected separately. The procedure was repeated twice.

The non-polar components were received in the pentane solution (EMMA), whereas more polar components were received in ethanol solution (EMMB). After separation, 270 mg of non-polar components was collected in pentane solution, and 4.191 g of polar components were collected in ethanol solution

### Example 4

### Spectroscopic Analysis of EMMA Extract

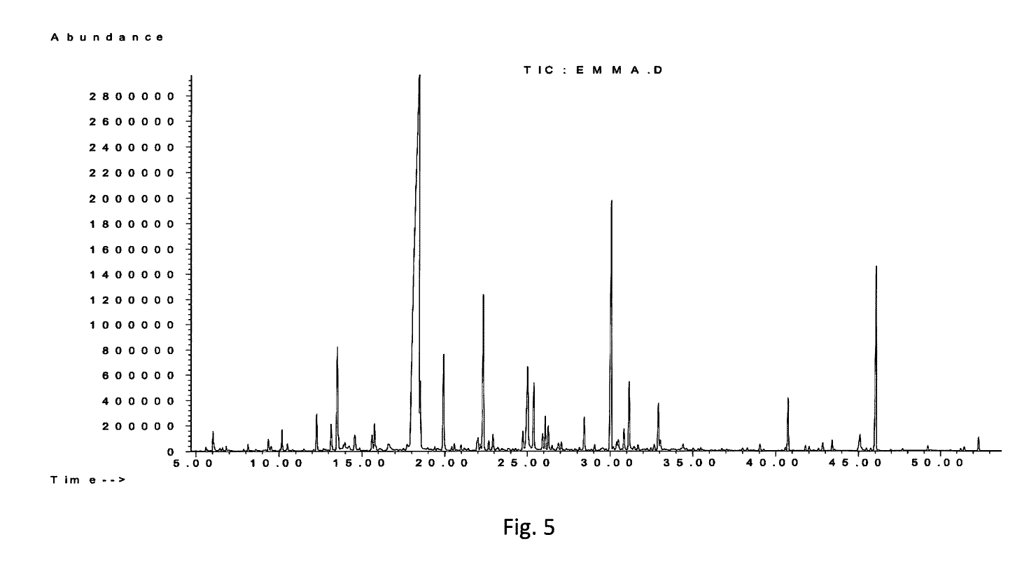

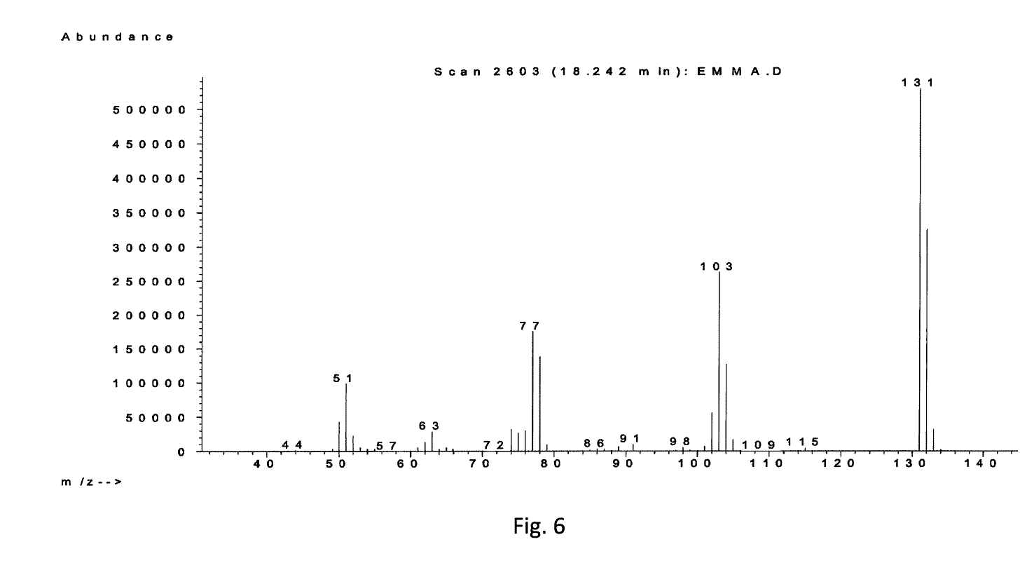

Spectroscopic analysis was performed on EMMA fraction as described in Example 3. In particular, GC-MS analysis showed that fraction EMMA contains benzoic acid ethyl ester, α-copaene, hexadecanoid acid ethyl ester and coumarin in small proportions, while the major metabolite is cinnamic aldehyde. The compounds were identified by comparing their spectral data (MS) and their retention time with those of standard samples. In particular, FIG. 5 shows GC-MS chromatogram of fraction EMMA and FIG. 6 shows mass spectrum of the major metabolite (cinnamic aldehyde)

### Example 5

### Nf-kB Activity of the EMM Extract and Related Fractions EMMA and EMMB

Fractions EMMA and EMMB obtained as described in Example 3, were subsequently tested for activation of NF-κB transcription factor that regulates the expression of pro-inflammatory genes. A variety of anti-inflammatory or anticarcinogenic phyto-chemicals suppress NF-κB signaling.

Samples for activity testing were prepared in ethanol and at concentrations adjusted to simulate the natural concentration in the originally provided extract. Table 1 here below shows the results of Fold of Nf-kB activation of two exemplary samples for activity testing.

TABLE 1

| | FOLD OF Nf-kB |
|---|---|
| SAMPLES | ACTIVATION |
| EMMB | 6.4 |
| EMMA + EMMB | 6.3 |

### Example 6

### Further Fractionation of EMM and EMMB and Nf-kB Activity of Related Subfractions

Fractions EMM and EMMB were subjected to further fractionations and the Nf-kB activity of the related subfractions measured

a. Size-Exclusion Chromatography of EMM Extract and Nf-kB of the Relevant Fractions.

Sephadex LH-20 was selected as the most appropriate resin for size-exclusion chromatography for small-sized molecules. In particular size-exclusion chromatography is based on the principle that exclusion of large molecules allows them to pass through more quickly and, while smallest molecules are retarded, an elude later separating the two molecules.

In particular, Sephadex LH-20 includes beaded, cross-linked dextran that has been hydroxypropylated to yield a chromatography medium with both hydrophilic and lipophilic character. Due to its dual character, Sephadex LH-20 swells in water and a number of organic solvents and is a liquid chromatography medium designed for molecular sizing of natural products.

EMM extract (0.9 g) was fractionated on a Sephadex LH-20 column (Column: 3×35 cm, Flow rate: 2.2 ml/min), using CH₂Cl₂/MeOH 50:50 with increasing amounts of MeOH, followed by increasing amounts of H₂O as the mobile phase, to yield 3 fractions (EMMS1-EMMS3). Table 2 here below shows such 3 fractions.

TABLE 2

| | | Collection | |
|---|---|---|---|
| | Fraction | polarity | Mass |
| 100 ml sample | S1 | CH₂Cl₂/MeOH = | 288.0 mg |
| (0.9 g) in | | 50/50 | |
| Sephadex | S2 | CH₂Cl₂/MeOH = | 480.0 mg |
| LH-20 | | 50/50 | |
| | S3 | MeOH/H₂O | 104.0 mg |

Samples for activity testing were prepared in ethanol and at concentrations adjusted to simulate the natural concentration in the originally provided extract. Table 3 shows the results of Fold of Nf-kB activation performed on such samples for activity testing.

TABLE 3

| | FOLD OF Nf-kB |
|---|---|
| SAMPLES | ACTIVATION |
| S1 | 1.2 |
| S2 | 1.1 |
| S3 | 1.1 |
| S1 + S2 + S3 | 2.0 |

Activity was lost, even in the “recombined” fraction, a fact that cannot be due to the stationary or mobile phase used in this protocol. The fractions were not processed any further.

b. Reversed Phase Vacuum Column Chromatography of EMM Extract and Nf-kB of the Related Fractions

A further experimental approach included reversed phase vacuum column chromatography performed on EMM extract. To avoid the acidic nature of silica gel used in normal phase chromatography, it was decided to fractionate EMM extract on reversed phase stationary phase.

EMM extract, concentrated to give a dark orange residue (180.0 mg), was subjected to reversed phase vacuum column chromatography, using H₂O with increasing amounts of MeOH as the mobile phase, to yield 3 fractions (EMMR1-EMMR3). Table 4 here below shows such 3 fractions.

TABLE 4

| | | Collection | |
|---|---|---|---|
| | Fraction | polarity | Mass |
| 20 ml EMM | R1 | H₂O/MeOH = | 121.0 mg |
| (180 mg) in | | 75/25 | |
| Reversed | R2 | H₂O/MeOH = | 34.0 mg |
| Phase VLC | | 75/25 | |
| (Sep-pak) | R3 | MeOH | 19.0 mg |

Samples for activity testing were prepared in ethanol and at concentrations adjusted to simulate the natural concentration in the originally provided extract.

Activity was lost, even in the “recombined” fraction. a fact that based on the experimental settings and verification used was excluded to the stationary or mobile phase used in this protocol. The fractions were not processed any further.

c. Isolation of Alkaloids from EMM Extract and Related Nf-kB Activity

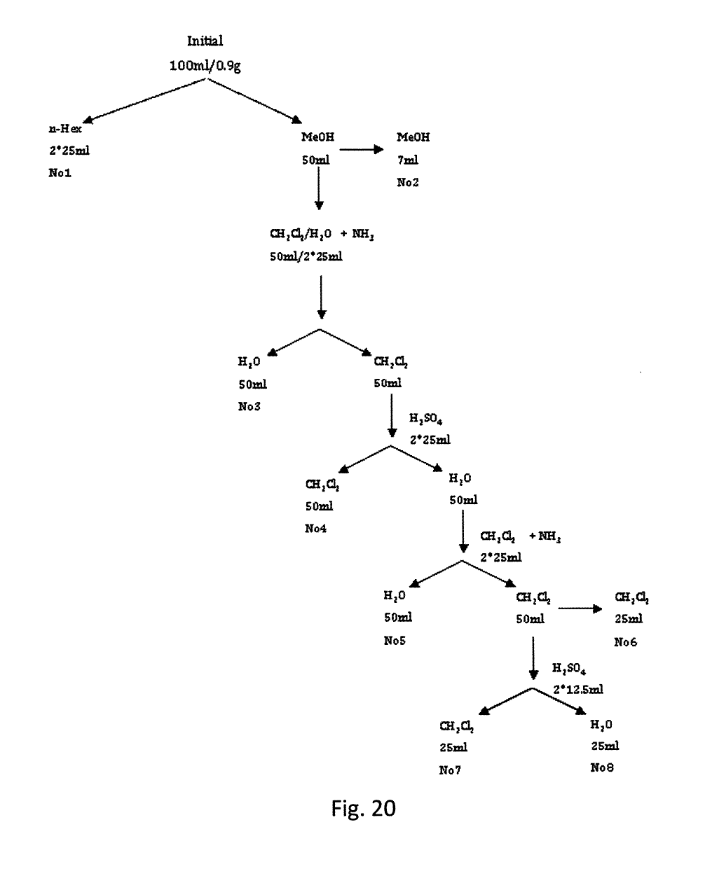

On the basis of the literature data available, it was possible that the extract might contain alkaloids. Therefore, a procedure based on extraction with two-phase liquid systems each time which is used to isolate alkaloids was applied to EMM extract (100 ml or 0.9 g) and is depicted in FIG. 20.

Samples for activity testing were prepared in ethanol and at concentrations adjusted to simulate the natural concentration in the originally provided extract.

TABLE 5

| | FOLD OF Nf-kB |
|---|---|
| SAMPLES | ACTIVATION |
| No1 | 1 |
| No2 | 1.2 |
| No3 | 7.9 |
| No4 | 1.3 |
| No5 | 1.1 |
| No6 | 1 |
| No7 | 1 |
| No8 | CELL TOXIC |
| 1 + 2 + 3 + 4 + | CELL TOXIC |
| 5 + 6 + 7 + 8 | |
| CRL+ | 10.4 |
| CRL− | 1 |

TABLE 6

| Fraction | Massa | |
|---|---|---|
| No1 | 40.0 | mg |
| No2 | 90.0 | mg |
| No3 | 610. | mg |
| No4 | 122. | mg |
| No5 | 40.0 | mg |
| No6 | 6 | mg |
| No7 | 25.0 | mg |
| No8 | 77 | mg |

Since fraction EMMN_{O}3 was the only active fraction, it was analyzed by ¹H NMR spectroscopy. Fraction EMMN_{O}3 lacks the majority of non-polar metabolites (fraction EMMN_{O}1 contains most of them) and of alkaloids (alkaloids were obtained in CH₂Cl₂ phase). Instead, the presence of sugars was detected.

To identify the sugars, a small quantity of fraction EMMN_{O}3 was acetylated and its reaction product was analyzed by GC-MS. Sucrose and glucose were identified as the major components of fraction EMMN_{O}3.

### c.1. Reversed Phase Vacuum Column Chromatography Performed on EMMN_{O}3

In order to clarify the presence of minor metabolites, EMMN_{O}3 fraction (610.0 mg) was subjected to reversed phase vacuum column chromatography, using H₂O with increasing amounts of MeOH as the mobile phase, to yield 3 fractions (EMMN_{O}3A-EMMN_{O}3C). The yield of the 3 fractions is shown in Table 7 here below.

TABLE 7

| | | Collection | |
|---|---|---|---|
| | Fraction | polarity | Mass |
| 610.0 mg No3 | No3A | H₂O | 564.0 mg |
| Reversed Phase | No3B | H₂O/MeOH = | 13.0 mg |
| VLC (sep-pak) | | 50/50 | |
| | No3C | MeOH | 40.0 mg |

Samples for activity testing were prepared in ethanol and at concentrations adjusted to simulate the natural concentration in the originally provided extract.

TABLE 8

| | FOLD OF Nf-kB |
|---|---|
| SAMPLES | ACTIVATION |
| 3A | 2.5 |
| 3B | 1 |
| 3C | 1.8 |
| 3A + 3B | 2.4 |
| 3A + 3C | 3.1 |
| 3B + 3C | 1.8 |
| 3A + 3B + 3C | 3.2 |

The fact that activity was lost, even in the “recombined” fraction, could perhaps be explained by the presence of traces of NH₃. The fractions were not processed any further.

d. Fractionation of EMM Extract by Successive Liquid-Liquid Extractions and Related Nf-kB Extract

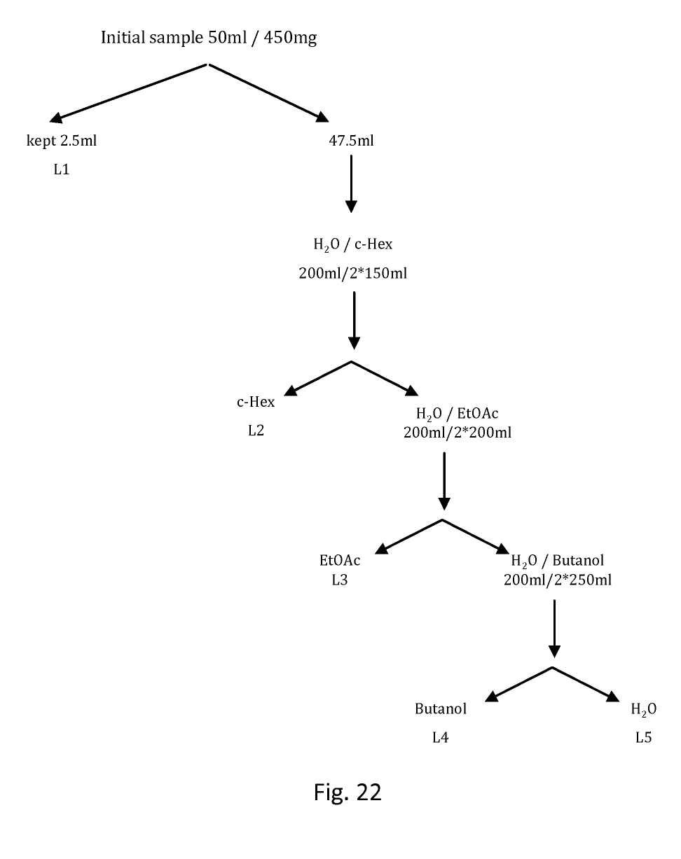

To avoid completely the use of any stationary phase, it was decided to fractionate EMM extract (50 mL/450 mg) by successive partitioning with two-phase liquid systems, as depicted in FIG. 22, to yield five fractions (EMML1-EMML5) which contain metabolites of different polarities.

Samples for activity testing were prepared in ethanol and at concentrations adjusted to simulate the natural concentration in the originally provided extract.

TABLE 9

| | FOLD OF Nf-kB |
|---|---|
| SAMPLES | ACTIVATION |
| L1 | 10.1 |
| L2 | 81.1 |
| L3 | 1.8 |
| L4 | 2.6 |
| L5 | 1.3 |
| L3 + L4 + L5 | 3.6 |

TABLE 10

| Fraction | Mass |
|---|---|
| L1 | 22.5 mg |
| L2 | 65.0 mg |
| L3 | 69. mg |
| L4 | 117. mg |
| L5 | 187.0 mg |

Because of the extremely high levels of activity exhibited by fraction EMML2 and the fact that it was identified as cinnamic aldehyde by ¹H NMR spectroscopy, it was decided to repeat the testing with the left-over material, as well as with new sample. Unfortunately, the activity was not observed again and the fractions were not processed any further.

e. EMMB Subtractions

Normal phase vacuum column chromatography was then performed on fraction EMMB. In particular, the ethanol residue (EMMB, 4 g) was subjected to vacuum column chromatography on silica gel, using cyclohexane with increasing amounts of EtOAc, followed by EtOAc with increasing amounts of MeOH as the mobile phase, to yield 5 fractions (EMMB1-EMMB5). Table 11 shows the 5 fractions.

TABLE 11

| Fraction | Collection polarity | Mass |
|---|---|---|
| B1 | c-Hex | 9.8 mg |
| B2 | C-Hex/EtOAc = 50/50 | 338.0 mg |
| B3 | EtOAc | 377.0 mg |
| B4 | C-Hex/MeOH = 50/50 | 1.24 g |
| B5 | MeOH | 1.97 g |

Fractions of EMMB so obtained, were subsequently tested for activation of NF-κB transcription factor that regulates the expression of pro-inflammatory genes. A variety of anti-inflammatory or anticarcinogenic phyto-chemicals suppress NF-κB signaling. Samples for activity testing were prepared in ethanol and at concentrations adjusted to simulate the natural concentration in the originally provided extract. Table 12 show the results of results of Fold of Nf-kB activation performed on such samples for activity testing.

TABLE 12

| | FOLD OF Nf-kB |
|---|---|
| SAMPLES | ACTIVATION |
| B3 | 1.4 |
| B4 | 1.3 |
| B5 | 1.6 |
| B3 + B4 | 2.3 |
| B3 + B5 | 2.5 |
| B3 + B4 + B5 | 3.6 |

The fact that activity was lost, even in the “recombined” fraction, supports the conclusion that some of the bioactive components might be sensitive to the stationary phase used in this protocol. The fractions were not processed any further.

### Example 7

### Further Analysis of the “Two Plant” Ethanolic Extract (EMM) or Agent X

Analysis of the“two-plant” ethanolic extract (EMM), or Agent-X, herein exemplified by a combination of ethanolic plant extract of a plant belonging to the genus Cinnamomum (plant 1), and a plant belonging to the genus of Jasminum (plant 2) was further analyzed through analysis of the individual extracts.

Plant 1 (plant belonging to the genus Cinnamomum) and plant 2 (plant belonging to the genus of Jasminum) ethanolic extracts were prepared from plant tissues that were provided in order to be evaluated for their NF-κB activation.

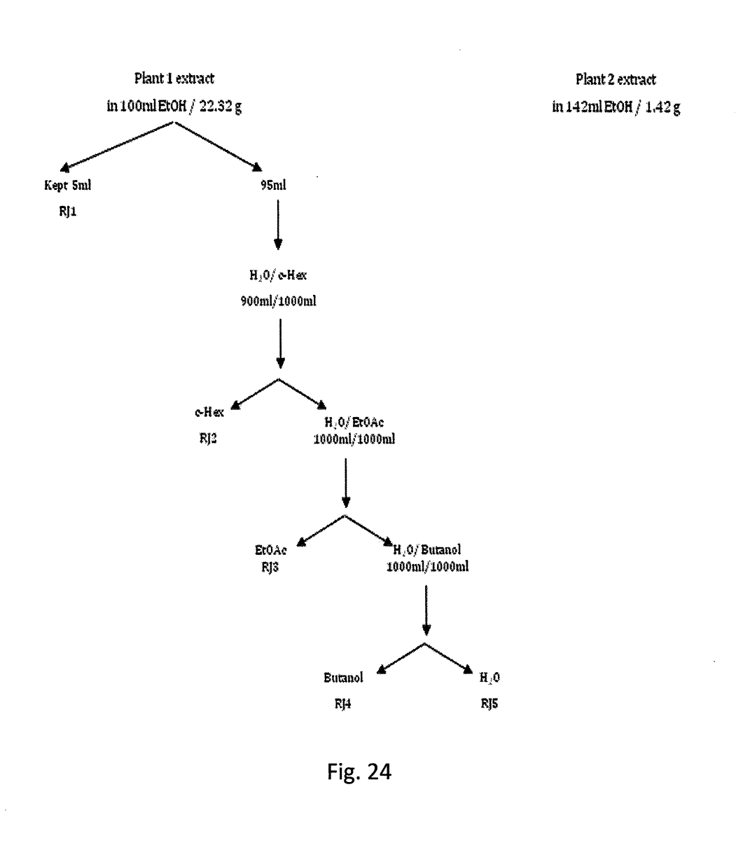

Plant 1 (22.32 g of plant 1 in 100 ml of EtOH) extract was further partitioned, as depicted in FIG. 24, with two-phase liquid systems to yield five fractions (RJ1-RJ5) which contain metabolites of different polarities. Plant 2 extract (1.42 g of plant 2 in 142 ml of EtOH) was not partitioned.

TABLE 13

| Fraction | Mass |
|---|---|
| RJ1 | 1.02 g |
| RJ2 | 4.28 mg |
| RJ3 | 810. mg |
| RJ4 | 3.71 g |
| RJ5 | 12.49 g |

Samples for activity testing were prepared in ethanol and at concentrations adjusted to simulate the natural concentration in the originally provided extract.

TABLE 14

| | FOLD OF Nf-kB |
|---|---|
| SAMPLES | ACTIVATION |
| RJ1 | CELL TOXIC |
| RJ2 | CELL TOXIC |
| RJ3 | CELL TOXIC |
| RJ4 | CELL TOXIC |
| RJ5 | CELL TOXIC |
| PLANT2 | 7.3 |
| RJ1 + PLANT2 | CELL TOXIC |
| RJ2 + PLANT2 | CELL TOXIC |
| RJ3 + PLANT2 | CELL TOXIC |
| RJ4 + PLANT2 | CELL TOXIC |
| RJ5 + PLANT2 | CELL TOXIC |
| RJ2 + RJ3 + RJ4 + | CELL TOXIC |
| RJ5 | |
| RJ2 + RJ3 + RJ4 + | CELL TOXIC |
| RJ5 + PLANT2 | |
| PLANT2 + 3 | CELL TOXIC |

Since the fractions of plant 1 extract were found to be cell toxic, the evaluation was repeated using 10-fold lower concentrations. The fractions continued to display toxicity on cells and therefore, they were not processed further. A possible explanation is that the provided plant tissues were not in flowering stage as the tissues were when used for the preparation of the EMM extract initially provided.

### Example 8

### Preparation of a Three-Plant Extract (EMMN) or Agent Y

The “three-plant” ethanolic extract (EMMN) or Agent Y including a ethanolic plant extract of the plant belonging to the genus Cinnamomum and the plant belonging to the genus of Jasminum, and a plant belonging to the genus Coffea hereafter referred to as “three-plant” ethanolic extract, was first prepared as follows.

Leaves, branches and flowers of the Jasmine in a flowering stage, cinnamon sticks and green coffee beans were placed in a jar in ethanol and then left at room temperature for 14 days. Ethanol was ethanol 95 and the analogy in volumes is: jasmine:ethanol 95=1:1.5, cinnamon: ethanol 95=1:14 and green coffee beans:ethanol 95=1:30 After the 14 days, the solution was drained and the rest of the plants removed.

### Example 9

### Analysis of a Three Plant Extract (EMMN) or Agent Y

Analysis of “three-plant” ethanolic extract (EMMN), or Agent-Y, prepared as described in Example 9, was performed as described below.

GC-MS analysis revealed that EMMN extract contains cinnamic aldehyde (9.4 min) and caffeine (16.8 min), while the major metabolite is coumarin (11.8 min). The compounds were identified by comparing their spectral data (MS) and their retention time with those of standard samples.

Coumarin is moderately toxic to the liver and kidneys, with an LD₅₀ value of 275 mg/kg. Alcoholic beverages sold in the European Union are limited to a maximum of 10 mg/L coumarin by law.

In order to quantify coumarin in EMMN extract, the latter was co-chromatographed with a known concentration of scopoletin used as internal standard (scopoletin is a chemical compound that is ionized in a similar way to coumarin). After the GC-MS analysis, the concentration of coumarin in EMMN extract was estimated at 0.17 mg/ml.

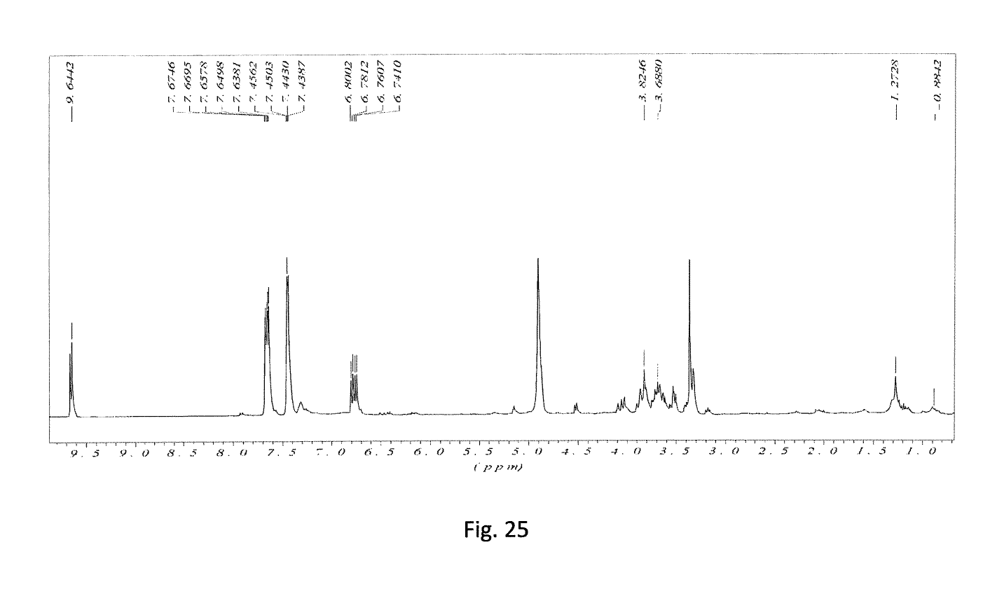

Reversed phase HPLC (High Pressure Liquid Chromatography) analysis of EMMN extract using a Diode Array Detector and a gradient elution solvent system (MeOH/H₂O) revealed the chromatogram shown on FIG. 25. Peaks 1-5 represent known natural products that were analyzed under the same chromatographic conditions.

Peak 1: chlorogenic acid, 7.4 min (sh 300, 326 nm)

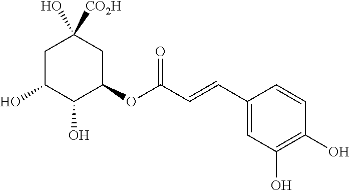 [SMILES: O=C(/C=C/c1ccc(O)c(O)c1)O[C@@H]1C[C@](O)(C(=O)O)C[C@@H](O)[C@H]1O]

Peak 2: coumarin, 22.0 min (277, 311 nm)

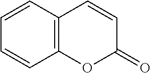 [SMILES: O=c1ccc2ccccc2o1]

Peak 3: 3,4-dihydroxy-hydrocinnamic acid, 22.6 min (283 nm)

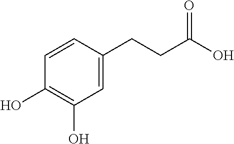 [SMILES: O=C(O)CCc1ccc(O)c(O)c1]

Peak 4: cinnamic aldehyde, 24.5 min (290 nm)

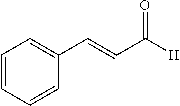

Peak 5: trans cinnamic acid, 24.7 min (273 nm)

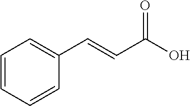 [SMILES: O=C(O)/C=C/c1ccccc1]

The total phenolic content (TPC) of EMMN extract was estimated, since phenolics may significantly contribute to its overall activity. TPC was determined using the Folin-Ciocalteu method. The reaction mixture contained 100 μl of sample extract, 200 μl of the Folin-Ciocalteu reagent, freshly prepared in our laboratory, 400 μl of saturated sodium carbonate and 9.3 ml of pure water. After two hours of reaction at ambient temperature, the absorbance at 765 nm was measured and used to calculate the phenolic contents using gallic acid as a standard. The EMMN extract had a total phenolic content of 6.2 mg GAE/g.

### Example 10

### Effect of Factor X on Basic Cellular Functions

Confirmation that factor-X does not affect basic cellular functions was shought. Factor X is a composition of 10 ingredients: avocado oil+cuivre de sulfate+ester vit C+PABA+Cod liver oil+Vit A+Vit D+EPA+DHA+Agent-X.

Agent-X is an ethanolic extract of the two plants, such as the ethanolic plant extract of the plant belonging to the genus Cinnamomum and of the plant belonging to the genus of Jasminum.

Factor-X does not affect basic cellular functions.

More in particular, based on hormonal, enzymatic and biochemical analyzes factor-X does not affect:
1. HORMONE-RECEPTORS INTERACTIONS: EGFa/b-EGFRa/b; CGH-CGHR; Melanocortin-MC 2/4ABR; Melanin concentrating hormone-MCHR; Melatonin-MTNR KeraA/C-KeraR; ADPcD-ADPcDR Histamine-HRH;
2. CELL CYCLE: Cell cycle control; Mitosis Mitogen FRs;
3. APOPTOSIS: Apoptosis EC; Apoptosis KC; Apoptosis AC;
4. CELLULAR SENESCENE: OXID/Tang; p53 signaling pathway; Ubiquitin signaling;
5. CELL RESPONSE AND PROTECTION: MAPK signaling; ErbB signaling; Wnt signaling; Notch signaling; TGF-B signaling; VEGF signaling; JAK/STAT signaling; mTOR signaling; Endocytosis; Lysosome; Peroxisome; Regulation of autophagy;
6. CELLULAR ADHESION & ELASTISITY: Focal adhesion; Adherens junction; Tight junction; Gap junction; Regulation of actin/collagen/elastin; Cell adhesion molecules (CAMs);
7. MELANOGENESIS & KERATINIZATION: Melanogenesis; Keratin synthesis; Melanocytes control; Keratinocytes control;
8. WATER & ION CYCLE; Hydration Pro/Re control; Ion/CONC/control Ca/K/Na/PO—; ABC transporters;
9. LIPID CYCLE: Membrane lipid control; Free lipid control; Adhesion lipid system;
10. PROTEINS & AMINO ACIDS CYCLE: Protein export system; Protein FLD control; Amino acids bio-cycle;
11. COFACTORS & VITAMINS CYCLE: Cofactors cycle RiBo-X; Cofactors cycle Biotin-X; Vitamin cycle RRDB/Retinol.

### Example 11

### Effect of Factor X on Cellular Metabolism

It was also confirmed that Factor-X does not affect the cellular metabolism. More in particular, based on hormonal, enzymatic and biochemical analyzes factor-X does not affect:

1. HORMONE-RECEPTORS INTERACTIONS: Leptin-LEPR; Ghrelin-GSHR; Orexin-HCRTR; ADPc-ADPcR; Epinephrine-ADR; HCAD-HCADR; Motilin-MLNR; Calcitonin-CALCR; Thyrotropin releasing hormone-TRHR; Triidothyronine thyroxine-THR; Growth hormone-GHR; GHRHC-GHRHCR; GIP-GIPR; INS/GLY-INSR/GLYcR; Cortisol-NR3C1 Serotonin-SEROcR;
2. CARBONHYDRATE METABOLISM: Glycolysis/Gluconeogenesis; Citrate cycle (TCA cycle); Pentose phosphate pathway; Pentose and glucuronate interconversions; Fructose and mannose metabolism; Galactose metabolism; Ascorbate and aldarate metabolism; Starch and sucrose metabolism; Amino sugar and nucleotide sugar metabolism; Pyruvate metabolism; Glyoxylate and dicarboxylate metabolism; Propanoate metabolism; Butanoate metabolism; Inositol phosphate metabolism;
3. ENERGY METABOLISM: Oxidative phosphorylation; Methane metabolism; Nitrogen metabolism; Sulfur metabolism;
4. LIPID METABILISM: Fatty acid biosynthesis; Fatty acid elongation in mitochondria; Fatty acid metabolism; Synthesis and degradation of ketone bodies; Steroid biosynthesis; Primary bile acid biosynthesis; Steroid hormone biosynthesis; Glycerophospholipid metabolism; Ether lipid metabolism; Sphingolipid metabolism; Arachidonic acid metabolism; Linoleic acid metabolism; Alpha-Linolenic acid metabolism; Biosynthesis of unsaturated fatty acids;
5. NUCLEOTIDE METABOLISM: Purine metabolism; Pyrimidine metabolism;
6. AMINO ACID METABOLISM: Alanine, aspartate and glutamate metabolism; Glycine, serine and threonine metabolism; Cysteine and methionine metabolism; Valine, leucine and isoleucine degradation; Valine, leucine and isoleucine biosynthesis; Lysine biosynthesis; Lysine degradation; Histidine metabolism; Tyrosine metabolism; Phenylalanine metabolism; Tryptophan metabolism; Phenylalanine, tyrosine and tryptophan biosynthesis;
7. METABOLISM OF OTHER AMINO ACIDS: Beta-Alanine metabolism; Taurine and hypotaurine metabolism; Selenoamino acid metabolism; Cyanoamino acid metabolism; D-Glutamine and D-glutamate metabolism; D-Arginine and D-ornithine metabolism; Glutathione metabolism;
8. METABOLISM & BIOSINTHESIS OF GLYCANS: N-Glycan biosynthesis; O-Glycan biosynthesis; O-Mannosyl glycan biosynthesis; Chondroitin sulfate biosynthesis; Heparan sulfate biosynthesis; Keratan sulfate biosynthesis; Glycosaminoglycan degradation; Glycosylphosphatidylinositol (GPI)-anchor biosynthesis; Glycosphingolipid biosynthesis-globo series; Glycosphingolipid biosynthesis-ganglio series; Other glycan degradation;
9. METABOLISM OF COFACTORS & VITAMINS: Thiamine metabolism; Riboflavin metabolism; Vitamin B6 metabolism; Nicotinate and nicotinamide metabolism; Pantothenate and CoA biosynthesis; Biotin metabolism; Lipoic acid metabolism; Folate biosynthesis; Retinol metabolism; Porphyrin and chlorophyll metabolism; Ubiquinone and other terpenoid-quinone biosynthesis;
10. SECONDARY METABOLITES BIOSYNTHESIS: Terpenoid backbone biosynthesis; Caffeine metabolism Limonene and pinene degradation.

### Example 12

### Molecular Analysis of Factor X

In the solution of Factor-X is not detected the presence of microorganisms and biological toxins over the usual and acceptable limits. More in particular, based on molecular analysis, in the solution of factor-X were not detected:

1. MICROORGANISMS: Acanthamoeba sp.; Acetobacter sp.; Acinetobacter baumannii; Actinomyces sp.; Actinomycetoma/Eumycetoma; Adenoviridae family; Aeromonas sp.; Agrobacterium sp.; Azorhizobium caulinodans; Azotobacter sp.; Anaplasma sp.; Ancylostoma sp.; Anisakis sp.; Arcanobacterium haemolyticum; Ascaris lumbricoides; Aspergillus genus; Astroviridae family; Babesia genus; Bacillus sp.; Bacteroides sp.; Balantidium sp.; Bartonella sp.; Baylisascaris genus; BK virus; Blastocystis hominis; Blastomyces dermatitidis; Bordetella sp.; Borrelia sp.; Brucella sp.; Bunyaviridae family; Burkholderia sp.; Caliciviridae family; Calymmatobacterium granulomatis; Campylobacter sp.; Candida sp.; Chlamydia sp.; Chlamydophila sp.; CJD prion; Clonorchis sinensis; Clostridium sp.; Coccidioides sp.; Colorado tick fever virus (CTFV); Corynebacterium sp.; Coxsackievirus; Coxiella sp.; Crimean-Congo hemorrhagic fever virus; Cryptococcus neoformans; Cryptosporidium genus; Cyclospora sp.; Cytomegalovirus; Dengue viruses (DEN-1, DEN-2, DEN-3, DEN-4); Dientamoeba fragilis; Diphyllobothrium; Dracunculus medinensis; Ebolavirus (EBOV); Echinococcus genus; Ehrlichia sp.; Entamoeba histolytica; Enterobacter cloacae; Enterobius vermicularis; Enterococcus sp.; Enterovirus genus; Enteroviruses; Epidermophyton floccosum/Trichophyton-rubrum/Trichophyton mentagrophytes Epstein-Barr Virus (EBV); Escherichia sp. Eustrongylides sp. Fasciola sp. Fasciolopsis buski FFI prion Filarioidea superfamily Flaviviruses; Fonsecaea pedrosoi; Francisella tularensis; Fusobacterium genus; Gardnerella vaginalis; Geotrichum candidum; Giardia sp.; Gnathostoma spinigerum; GSS prion; Guanarito virus; H1N1 virus; Haemophilus sp.; Halobaena caerulea; Helicobacter pylori; Hepatitis A Virus; Hepatitis B Virus; Hepatitis C Virus; Hepatitis D Virus; Hepatitis E Virus; Herpes simplex virus 1/2 (HSV-1/HSV-2); Histoplasma capsulatum; HIV (Human immunodeficiency virus); Hortaea werneckii; Human bocavirus (HBoV); Human cytomegalovirus; Human herpesvirus 6 (HHV-6); Human herpesvirus 7 (HHV-7)/+8; Human metapneumovirus (hMPV); Human papillomavirus (HPV); Human parainfluenza viruses (HPIV); Hymenolepis sp.; Isospora belli; Influenza virus; JC virus; Junin virus; Kingella sp.; Klebsiella sp.; Kuru prion; Lactobacillus sp.; Lactococcus lactis; Lassa virus; Legionella pneumophila; Leishmania genus; Leptospira genus; Listeria sp.; Lymphocytic choriomeningitis virus (LCMV); Machupo virus; Malassezia genus; Marburg virus; Measles virus; Metagonimus yokagawai; Methanobacterium sp.; Microbacterium sp.; Micrococcus luteus; Microsporidia phylum; Miscellaneous sp.; Molluscum contagiosum virus (MCV); Moraxella catarrhalis; Mucorales order (Mucormycosis)/Entomophthorales; Mumps virus Mycobacterium sp.; Mycoplasma sp.; Naegleria fowleri; Nanophyetus sp.; Neisseria sp.; Nocardia sp.; Norovirus; Onchocerca volvulus; Orthomyxoviridae family; Pachyptila sp./itm.; Papillomavirus; Paracoccidioides brasiliensis; Paragonimus westermani/Paragonimus species; Parainfluenza virus; Parvovirus B19; Pasteurella sp.; Pediculus humanus capitis; Peptostreptococcus Phthirus pubis; Piedraia hortae; Plasmodium genus; Plesiomonas shigelloides; Pneumocystis jirovecii; Poliovirus; Porphyromonas gingivalis; Prevotella genus; Pseudomonas aeruginosa; Rabies virus; Respiratory syncytial virus (RSV); Rhizobium radiobacter; Rhinosporidium seeberi; Rhinoviruses/Coronaviruses; Rickettsia sp.; Rift Valley fever virus; Rochalimaea sp.; Rotavirus; Rothia dentocariosa; Rubella virus; Sabia Salmonella sp.; Sarcocystis hominis; Sarcoptes scabiei; SARS coronavirus; Schistosoma genus; Serratia marcescens; Shigella sp.; Sin Nombre virus Sporothrix schenckii; Staphylococcus sp.; Stenotrophomonas maltophilia; Streptobacillus moniliformis; Streptococcus sp.; Strongyloides stercoralis; Taenia genus; Taenia solium; Toxocara canis/Toxocara cati; Toxoplasma sp.; Treponema sp.; Trichinella sp.; Trichomonas vaginalis; Trichophyton genus; Trichosporon beigelii; Trichuris trichiura; Trypanosoma sp.; Ureaplasma urealyticum; Varicella zoster virus (VZV); Variola major/Variola minor; vCJD prion; Venezuelan equine encephalitis virus; Vibrio sp.; West Nile virus; Wolbachia; Wuchereria sp.; Yellow fever virus; Yersinia sp.
2. BIOLOGICAL TOXINS: 3-Nitropropionic acid; Aflatoxins; Alkaloids; Bacillus cereus; Ciguatera poisoning; Citreoviridin; Clostridium botulinum; Clostridium perfringens; Cyclopiazonic acid; Cytochalasins; Ergopeptine alkaloids; Ergot alkaloids; Ergotamine; Foxglove; Fumonisins; Fusaric acid; Fusarochromanone; Gempylotoxin; Grayanotoxin; Kojic acid; Lolitrem alkaloids; Moniliformin; Mushroom toxins; Nivalenol; Ochratoxins; Oosporeine; Patulin; Phomopsins; Phytohaemagglutinin; Poisonous hemlock; Pyrrolizidine alkaloids; Scombrotoxin; Shellfish toxin; Sporidesmin A; Staphylococcus aureus; Sterigmatocystin; Tetrodotoxin; Tremorgenic mycotoxins; Trichothecenes; Zearalenols.

### Example 13

### GMO Analysis of Factor X

In the solution of factor-X are not detected GMOs. More in particular, based on molecular analysis, in the solution of factor-X are not detected:

GMO FRAMES: CaMVp35S; T-nos sp/ITM; Bt11; Bt176; Bt4332/pQ4511; CMVprom012; MON810plus; QP35S SP/ITMs; cDNAmutR112; cDNAmutF112; cENHCmutR122; cENHCmutF122; cORImutR1131; cORImutF1131; cAUGmutTER/R/F0542; ELcdpT4; ELcd122/77; ELrASIA/09; ELrAFRC/09; ELrEE/010; ELrUS/010; EDNA/09; CcDNApbrWQ11; qR/Fori; pR/Fter.

### Example 14

### Effect of Biological Activity of Factor-X

It was proved that factor-X activates the transcription factor NF-kB by 10 times increasing time of cell exposure to factor-X leads to increase the activation of the transcription factor NF-kB by 10 times (1012%) with a maximum effect at 9 to 12 hours in epithelial cells and B-cells.

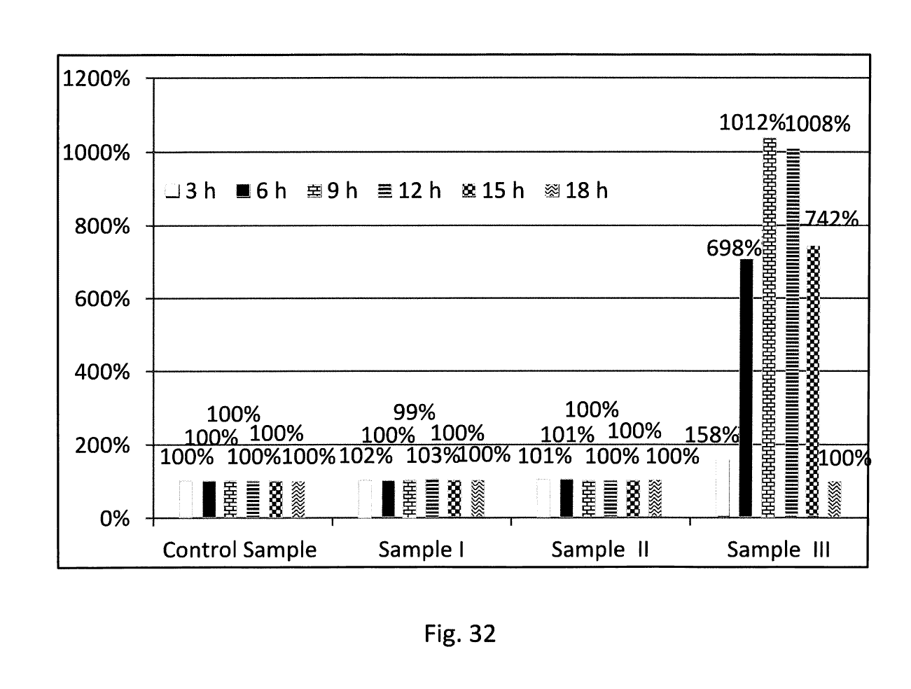

In particular, FIG. 32 shows a comparison of results among Sample I, Sample II and Sample III. In particular in the experiments leading to the results of FIG. 32,
1) the basic components of the solution of factor-X are: ester vitC, PABA, cod liver oil, vitA, vitD, EPA+DHA,
2) the liquids are: avocado oil and copper sulfate and
3) agent-X is: the 2-plant ethanolic extract.

SAMPLE I: contains the basic components of the solution of factor-X, without the liquids and agent X; SAMPLE II: contains the basic components of the solution of factor-X, the liquids without the agent-X; SAMPLE III: contains the basic components of the solution of factor-X. with the liquids and agent-X.

Increasing of the transcription factor NF-kB activation up to 10 times with increasing time of exposure of the cells to factor-X with maximum effect from 9 to 12 hours can be achieved by the synergy of agents in factor-X solution (sample III)

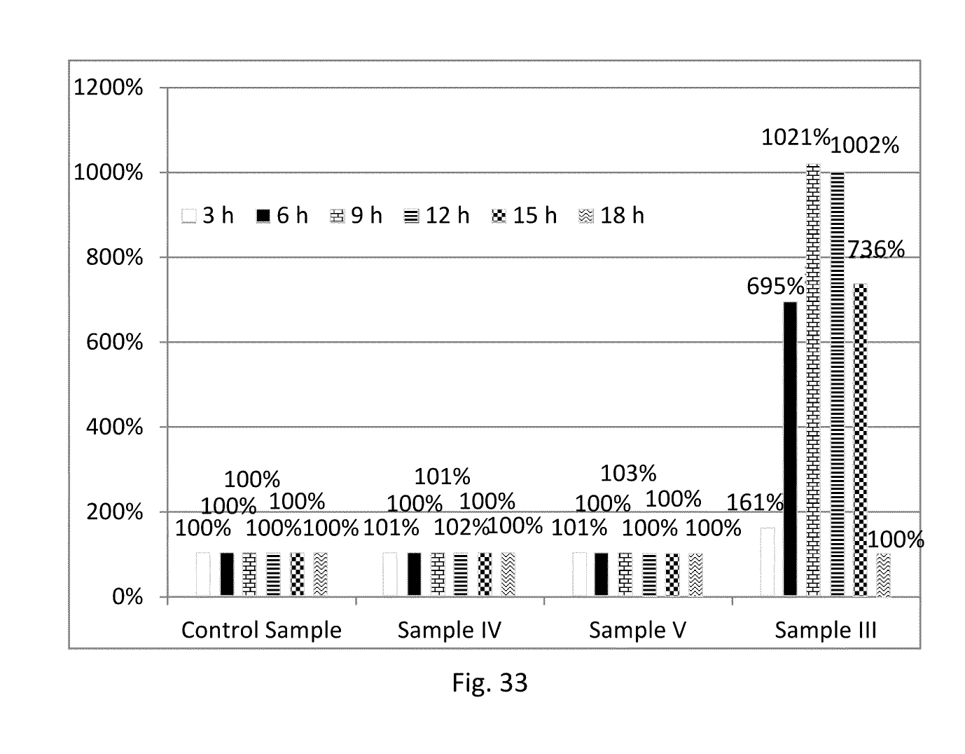

FIG. 33 shows a comparison of results among Sample IV, Sample V and Sample III. in the experiments leading to the results of FIG. 33
1) the basic components of the solution of factor-X are: ester vitC, PABA, cod liver oil, vitA, vitD, EPA+DHA,
2) the liquids are: avocado oil and copper sulfate and
3) agent-X is: the 2-plant ethanolic extract.

SAMPLE III: contains the basic components of the solution of factor-X. with the liquids and agent-X. SAMPLE IV: contains no basic constituents of the solution of factor-X and no agent-X, only liquids and SAMPLE V: contains only the agent-X without the basic constituents of the solution of factor-X, without the liquids.

### Example 15

### Effect of Biological Activity of Factor-X

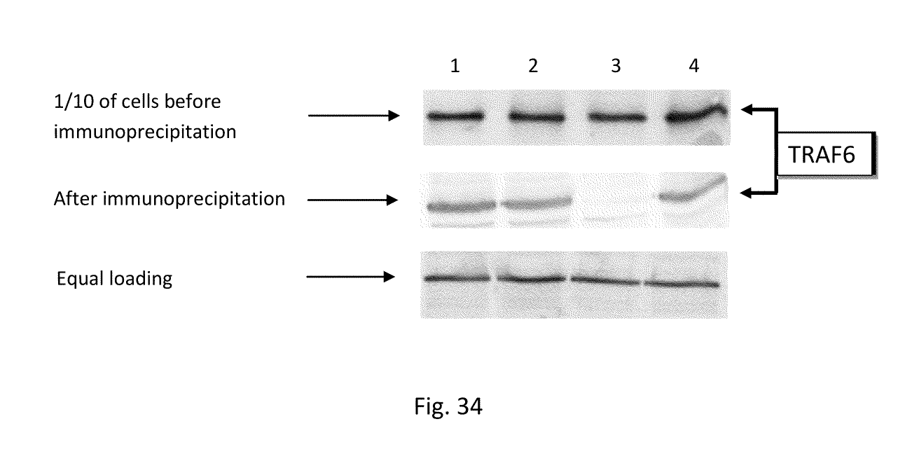

Factor-X blocks the binding of protein TRAF6 to signal transduction pathway that begins from the permanently activated CD40 in epithelial cells and B-lymphocytes mimics pathological autoimmune diseases. In particular, FIG. 34 shows immunoprecipitation of the complex based TRAFsfml-TRAF6 induced by activated cytoplasmic domain of the receptor CD40. More in particular, in the experiments leading to the results of FIG. 34,
1) the basic components of the solution of factor-X are: ester vitC, PABA, cod liver oil, vitA, vitD, EPA+DHA,
2) the liquids are: avocado oil and copper sulfate and
3) agent-X is: the 2-plant ethanolic extract.

SAMPLE I: contains the basic components of the solution of factor-X, without the liquids and agent-X; SAMPLE II: contains the basic components of the solution of factor-X, the liquids without the agent-X (“two plant” ethanolic extract); SAMPLE III: contains the basic components of the solution of factor-X with the liquids and agent-X (“two plant” ethanolic extract), Control Sample

The results shown in FIG. 34 shows that this phenomenon is achieved only by the synergy of agents of the solution of factor-X (Sample III).

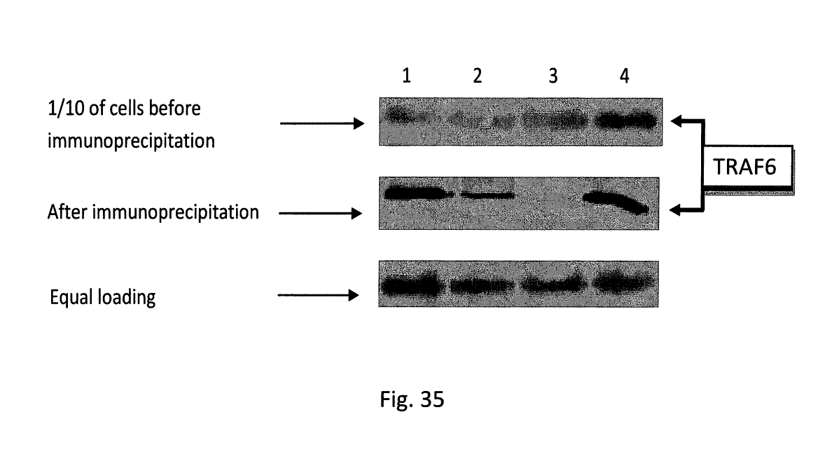

FIG. 35 immunoprecipitation of the complex based TRAFsfml-TRAF6 induced by activated cytoplasmic domain of the receptor CD40 is shown. In particular in the experiments leading to the results of FIG. 35:
1) the basic components of the solution of factor-X are: ester vitC, PABA, cod liver oil, vitA, vitD, EPA+DHA,
2) the liquids are: avocado oil and copper sulfate and
3) agent-X is: the 2-plant ethanolic extract. SAMPLE III: contains the basic components of the solution of factor-X, the liquids and agent-X. SAMPLE IV: contains no basic constituents of the solution of factor-X and no agent-X, only liquids and SAMPLE V: contains only the agent-X without the basic constituents of the solution of factor-X, without the liquids. Control Sample

It was further confirmed that factor-X does not affect the normal growth and health of wild type mice. To study such effect of factor-X in mice, were organized the following groups of animals.

20 wild type mice (wt C57/C57) 3 months old were divided into two groups of 10 mice each. The first group named A and received along with the food the solution of the factor-X every 12 hours per day for three weeks while the second group B received placebo solution likewise with food daily every 12 hours for three weeks. After the end of treatment showed no difference in the growth and health of the mice of both groups A and B. Specifically mice were studied for group A and group B as identified in table 15 here below.

TABLE 15

| | Group A | Group B |
|---|---|---|
| Weight | Normal | Normal |
| complete blood tests | Normal | Normal |
| basic biochemical analyzes | Normal | Normal |
| examination by veterinary | Normal | Normal |
| specialists in basic | | |
| physiological functions | | |

Factor-X eliminates the presence of autoantibodies against DNA of the thymus of transgenic mice LMP1/CD40. To study such effect of factor-X in mice, were organized the following groups of animals:

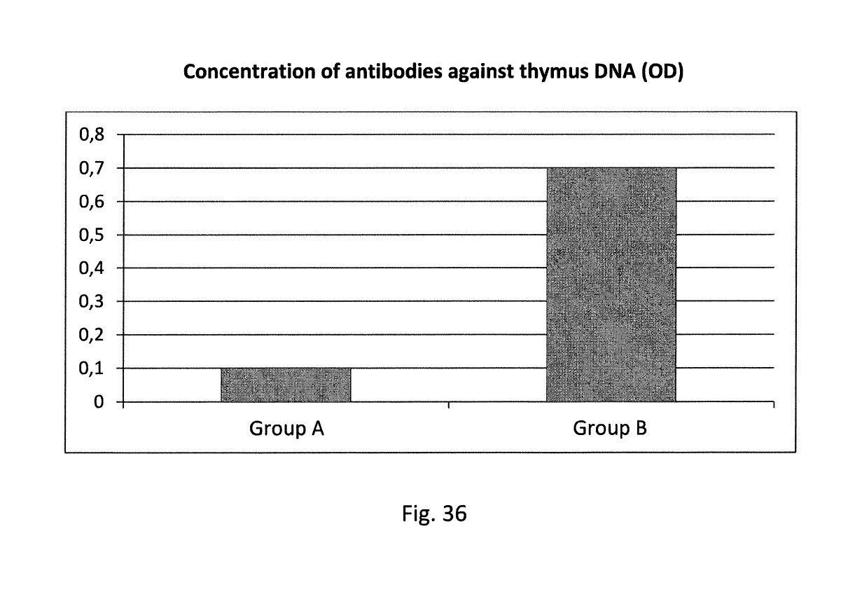

20 LMP1/CD40 transgenic mice 3 months old were divided into two groups of 10 mice each. The first group named A and received along with the food the solution of the factor-X every 12 hours per day for three weeks while the second group B received placebo solution likewise with food daily every 12 hours for three weeks. In serum of the animals detect antibodies against thymus DNA by the method of ELISA and found that the transgenic mice of group B LMP1/CD40 have autoantibodies against thymus DNA in serum at much higher concentration than the mice of Group A. The results of concentration of antibodies against thymus DNA (OD) are shown in diagram of FIG. 36. Detection of antibodies against thymus DNA by the method of ELISA in mice sera. The statistical analysis was performed using Student control method and specifically by applying two-tailed unpaired Student t test (p<0.001).

### Example 16

### Effect of Biological Activity of Factor-X

Factor-X eliminates the deposition of immunoglobulin IgG in the kidney of transgenic mice LMP1/CD40. To study such effect of factor-X in mice, were organized the following groups of animals.

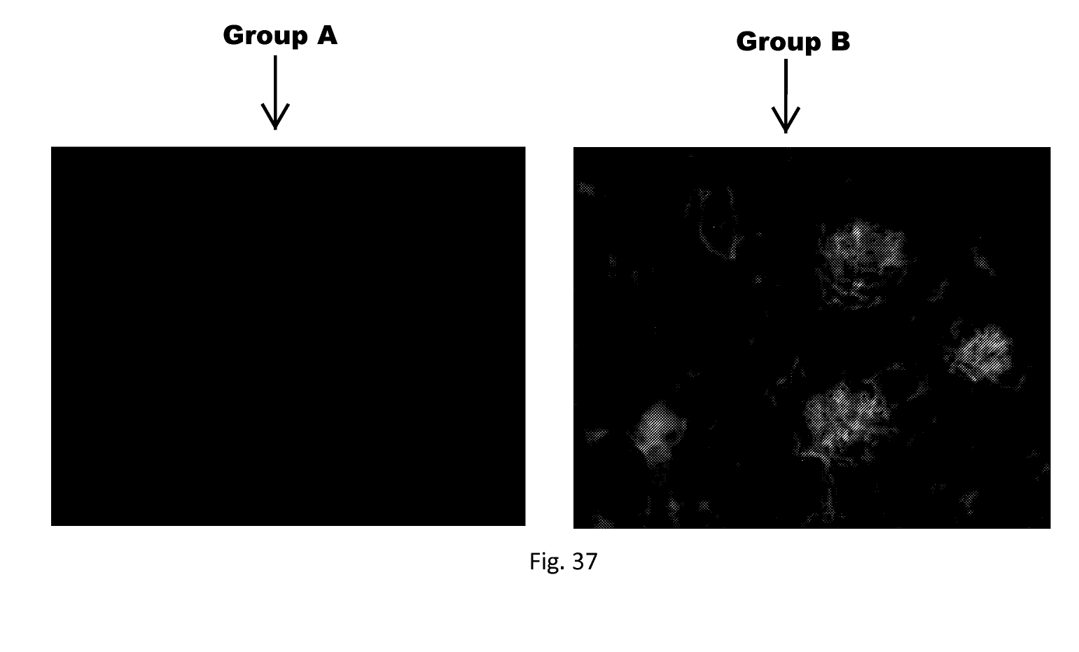

20 LMP1/CD40 and transgenic mice 3 months old were divided into two groups of 10 mice each. The first group named A and received along with the food the solution of the factor-X every 12 hours per day for three weeks while the second group B received placebo solution likewise with food daily every 12 hours for three weeks. A fuller and more convincing observation of eliminating the existence of autoantibodies in transgenic mice LMP1/CD40 is the detection of immunoglobulins IgG deposits in the kidneys of mice. Specifically from mice were isolated kidneys and made incisions and detection of immunoglobulins IgG mouse with fluorescence. The sections revealed the presence of the immunoglobulins IgG deposits in the kidney of transgenic mice LMP1/CD40 group B, which are absent from the sections of the transgenic mice of Group A. FIG. 37 shows Kidney sections and detection of immunoglobulins IgG mouse with fluorescence.

### Example 17

### Effect of Biological Activity of Factor-X

Factor-X eliminates perivascular liver inflammation of transgenic mice LMP1/CD40. To study such effect of factor-X in mice, were organized the following groups of animals.

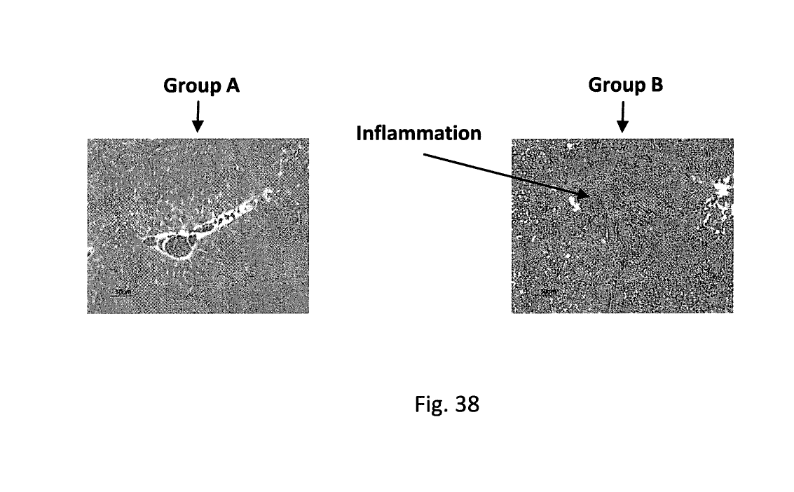

20 LMP1/CD40 and transgenic mice 3 months old were divided into two groups of 10 mice each. The first group named A and received along with the food the solution of the factor-X every 12 hours per day for three weeks while the second group B received placebo solution likewise with food daily every 12 hours for three weeks. Liver sections from transgenic mice LMP1/CD40 incubated with hematoxylin-eosin and observed that in sections of the transgenic mice of group A are not observed perivascular inflammation, which are particularly common in sections of the transgenic mice of group B. FIG. 38 shows Sections of liver of transgenic mice LMP1/CD40 hematoxylin-eosin staining.

### Example 18

### Effect of Biological Activity of Factor-X

Factor-X does not eliminate all the characteristics of abnormal functioning of the immune system of transgenic mice LMP1/CD40. To study such effect of factor-X in mice, were organized the following groups of animals.

20 LMP1/CD40 and transgenic mice 3 months old were divided into two groups of 10 mice each. The first group named A and received along with the food the solution of the factor-X every 12 hours per day for three weeks while the second group B received placebo solution likewise with food daily every 12 hours for three weeks. The solution of the factor-X does not eliminate all the characteristics of abnormal functioning of the immune system of transgenic mice LMP1/CD40. A comparison between group A and group B is shown in Table 16 here below.

TABLE 16

| Group A | Group B |
|---|---|
| Elevated levels of macrophage | Elevated levels of macrophage |
| Elevated levels of polymorphonuclear neutrophils | Elevated levels of polymorphonuclear neutrophils |
| Elevated levels of CD4 + T-lymphocyte | Elevated levels of CD4 + T-lymphocyte |
| Elevated levels of CD8 + T-lymphocytes | Elevated levels of CD8 + T-lymphocytes |
| Elevated levels of CD69 | Elevated levels of CD69 |
| Elevated levels of CD80 | Elevated levels of CD80 |
| Elevated levels of CD86 | Elevated levels of CD86 |
| Elevated levels of non-specific IgM | Elevated levels of non-specific IgM |
| Reduced levels of specific antibodies against pathogen | Reduced levels of specific antibodies against pathogen |
| Suppression forming ability of germinal centers | Suppression forming ability of germinal centers |
| Reduced affinity antibodies specific for the | Reduced affinity antibodies specific for the immunogen |
| immunogen lowered affinity | lowered affinity antibodies |

### Example 19

### Effect of Biological Activity of Factor-X

Factor-X increases the biological activity and reactivity of the metabolism of tyrosine in groups of mice VTLG-C57/J6 (vitiligo mouse model) and in groups of wild-type mice. To study the effect of factor-X in mice, were organized the following groups of animals.
1. 5 groups of mice VTLG-C57/J6 (vitiligo mouse model)—20 animals each called Group A.
2. 5 groups of mice VTLG-C57/J6 (vitiligo mouse model)—20 animals each Group B.
3. 5 groups of wild-type mice—20 animals each called Group Γ.
4. 5 groups of wild-type mice—20 animals each called Group A.
5. 5 groups of mice AIA-C57/C57 (arthritis mouse model)—20 animals each called Group AA.
6. 5 groups of mice AIA-C57/C57 (arthritis mouse model)—20 animals each called BB Group.
7. 5 groups of wild-type mice—20 animals each called the ΓΓ Group.
8. 5 groups of wild-type mice—20 animals each called Group ΔΔ.

At time 0 the study begins with the administration in food daily and once every 12 hours of the following solutions:

Normal solution of factor-X: Group A; Group Γ; Group AA; Group ΓΓ and Placebo solution without factor-X: Group B; Group Δ; Group BB; Group ΔΔ.

Each group, e.g. group A, includes 5 groups of 20 animals each, wherein said administration is carried out for a period of 1, 2, 3, 4 and 5 months, thus forming subsets A1, A2, A3, A4 and A5. The same treatment was for the other groups B, Γ, Δ, AA, BB, ΓΓ and ΔΔ. For example, Biological Activity and Biological Reactivity were analyzed for Subgroup A4; Subgroup A5; Subgroup Γ4; Subgroup Γ5; Subgroup ΓΓ4; Subgroup ΓΓ5.

The results of such analysis can be summarized as follows:
Subgroup A4: Tyrosine metabolism—Mus musculus (mouse) shown 164% of Biological Activity and 132% of Biological Reactivity.
Subgroup A5: Tyrosine metabolism—Mus musculus (mouse) shown 197% of Biological Activity and 134% of Biological Reactivity.
Subgroup Γ4: Tyrosine metabolism—Mus musculus (mouse) shown 201% of Biological Activity and 142% of Biological Reactivity.
Subgroup Γ5: Tyrosine metabolism—Mus musculus shown 255% of Biological Activity and 136% of Biological Reactivity.
Subgroup ΓΓ4: Tyrosine metabolism—Mus musculus (mouse) shown 205% of Biological Activity and 138% of Biological Reactivity.
Subgroup ΓΓ5: Tyrosine metabolism—Mus musculus (mouse) shown 253% of Biological Activity and 138% of Biological Reactivity.

### Example 20

### Effect of Biological Activity of Factor-X

Factor-X increases the biological activity and reactivity of melanogenesis in groups of mice VTLG-C57/J6 (vitiligo mouse model) and in groups of wild-type mice To study the effect of factor-X in mice, were organized the following groups of animals:
1. 5 groups of mice VTLG-C57/J6 (vitiligo mouse model)—20 animals each called Group A.
2. 5 groups of mice VTLG-C57/J6 (vitiligo mouse model)—20 animals each Group B.
3. 5 groups of wild-type mice—20 animals each called Group Γ.
4. 5 groups of wild-type mice—20 animals each called Group A.
5. 5 groups of mice AIA-C57/C57 (arthritis mouse model)—20 animals each called Group AA.
6. 5 groups of mice AIA-C57/C57 (arthritis mouse model)—20 animals each called BB Group.
7. 5 groups of wild-type mice—20 animals each called the ΓΓ Group.
8. 5 groups of wild-type mice—20 animals each called Group ΔΔ.

At time 0 the study begins with the administration in food daily and once every 12 hours of the following solutions:

Normal solution of factor-X: Group A; Group Γ; Group AA; Group ΓΓ and placebo solution without factor-X: Group B; Group Δ; Group BB; Group ΔΔ.

Each group, e.g. group A, includes 5 groups of 20 animals each, wherein said administration is carried out for a period of 1, 2, 3, 4 and 5 months, thus forming subsets A1, A2, A3, A4 and A5. The same treatment was for the other groups B, Γ, Δ, AA, BB, ΓΓ and ΔΔ.

The results of such analysis can be summarized as follows:
Subgroup A4: Metabolic Process Melanogenesis—Mus musculus (mouse) shown 152% of Biological Activity and 141% of Biological Reactivity.
Subgroup A5: Metabolic Process Melanogenesis—Mus musculus (mouse) shown 214% of Biological Activity and 144% of Biological Reactivity.
Subgroup Γ4: Metabolic Process Melanogenesis—Mus musculus (mouse) shown 181% of Biological Activity and 140% of Biological Reactivity.
Subgroup Γ5: Metabolic Process Melanogenesis—Mus musculus (mouse) shown 242% of Biological Activity and 141% of Biological Reactivity.
Subgroup ΓΓ4: Metabolic Process Melanogenesis—Mus musculus (mouse) shown 177% of Biological Activity and 142% of Biological Reactivity.
Subgroup ΓΓ5: Metabolic Process Melanogenesis—Mus musculus (mouse) shown 244% of Biological Activity and 144% of Biological Reactivity.

### Example 21

### Effect of Biological Activity of Factor-X

Factor-X increases the biological activity of serotonergic synapses groups of mice VTLG-C57/J6 (vitiligo mouse model), in groups of wild type mice and in groups of mice AIA-C57/C57 (arthritis mouse model).

To study the effect of factor-X in mice, were organized the following groups of animals:
1. 5 groups of mice VTLG-C57/J6 (vitiligo mouse model)—20 animals each called Group A.
2. 5 groups of mice VTLG-C57/J6 (vitiligo mouse model)—20 animals each Group B.
3. 5 groups of wild-type mice—20 animals each called Group Γ.
4. 5 groups of wild-type mice—20 animals each called Group Δ.
5. 5 groups of mice AIA-C57/C57 (arthritis mouse model)—20 animals each called Group AA.
6. 5 groups of mice AIA-C57/C57 (arthritis mouse model)—20 animals each called BB Group.
7. 5 groups of wild-type mice—20 animals each called the ΓΓ Group.
8. 5 groups of wild-type mice—20 animals each called Group ΔΔ.

At time 0 the study begins with the administration in food daily and once every 12 hours of the following solutions:

Normal solution of factor-X: Group A; Group Γ; Group AA; Group ΓΓ and placebo solution without factor-X: Group B; Group Δ; Group BB; Group ΔΔ.

Each group e.g. group A includes 5 groups of 20 animals each, wherein said administration is carried out for a period of 1, 2, 3, 4 and 5 months, thus forming subsets A1, A2, A3, A4 and A5. The same treatment was for the other groups B, Γ, Δ, AA, BB, ΓΓ and ΔΔ.

The results of such analysis can be summarized as follows:
Subgroup A4: Metabolic Process Serotonergic synapse—Mus musculus (mouse) shown 123% of Biological Activity and 100% of Biological Reactivity.
Subgroup A5: Metabolic Process Serotonergic synapse—Mus musculus (mouse) shown 125% of Biological Activity and 100% of Biological Reactivity.
Subgroup Γ4: Metabolic Process Serotonergic synapse—Mus musculus (mouse) shown 122% of Biological Activity and 100% of Biological Reactivity.
Subgroup Γ5: Metabolic Process Serotonergic synapse—Mus musculus (mouse) shown 125% of Biological Activity and 100% of Biological Reactivity.
Subgroup ΓΓ4: Metabolic Process Serotonergic synapse—Mus musculus (mouse) shown 122% of Biological Activity and 100% of Biological Reactivity.
Subgroup ΓΓ5: Metabolic Process Serotonergic synapse—Mus musculus (mouse) shown 123% of Biological Activity and 100% of Biological Reactivity.
Subgroup AA4: Metabolic Process Serotonergic synapse—Mus musculus (mouse) shown 122% of Biological Activity and 100% of Biological Reactivity.
Subgroup AA5: Metabolic Process Serotonergic synapse—Mus musculus (mouse) shown 123% of Biological Activity and 100% of Biological Reactivity.

### Example 22

### Effect of Biological Activity of Factor-X

Factor-X increases the expression of NGF (Nerve Growth Factor) in groups of mice VTLG-C57/J6 (vitiligo mouse model), in groups of wild type mice and in groups of mice AIA-C57/C57 (arthritis mouse model)

To study the effect of factor-X in mice, were organized the following groups of animals:
1. 5 groups of mice VTLG-C57/J6 (vitiligo mouse model)—20 animals each called Group A.
2. 5 groups of mice VTLG-C57/J6 (vitiligo mouse model)—20 animals each Group B.
3. 5 groups of wild-type mice—20 animals each called Group Γ.
4. 5 groups of wild-type mice—20 animals each called Group Δ.
5. 5 groups of mice AIA-C57/C57 (arthritis mouse model)—20 animals each called Group AA.
6. 5 groups of mice AIA-C57/C57 (arthritis mouse model)—20 animals each called BB Group.
7. 5 groups of wild-type mice—20 animals each called the ΓΓ Group.
8. 5 groups of wild-type mice—20 animals each called Group ΔΔ.

At time 0 the study begins with the administration in food daily and once every 12 hours of the following solutions:

Normal solution of factor-X: Group A; Group Γ; Group AA; Group ΓΓ, and Placebo solution without factor-X

Each group e.g. group A includes 5 groups of 20 animals each, wherein said administration is carried out for a period of 1, 2, 3, 4 and 5 months, thus forming subsets A1, A2, A3, A4 and A5. The same treatment was for the other groups B, Γ, Δ, AA, BB, ΓΓ and ΔΔ.

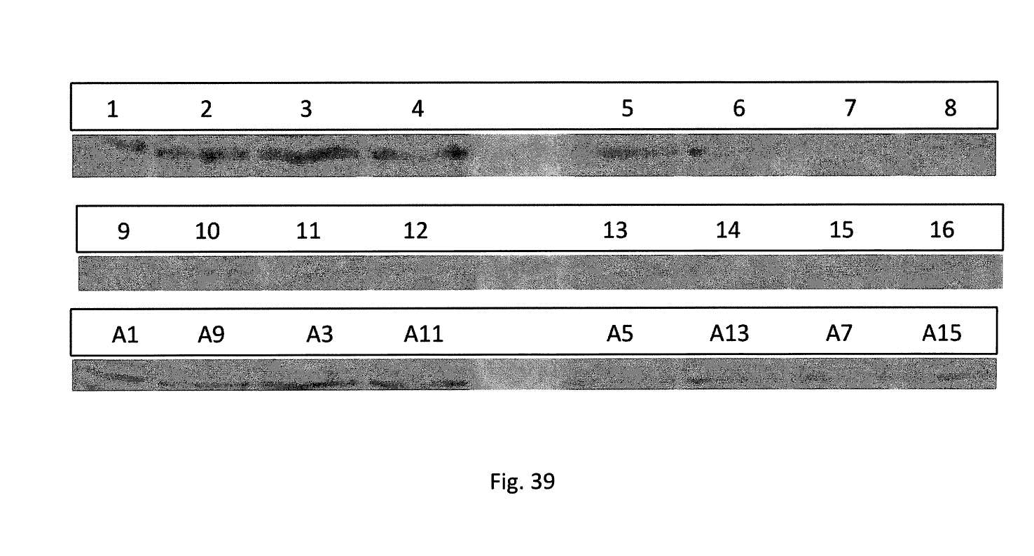

FIG. 39 shows immunoprecipitations of subgroups, wherein Subgroup A4 corresponds to 1; Subgroup A5 corresponds to 2; Subgroup Γ4 corresponds to 3; Subgroup Γ5 corresponds to 4; Subgroup B4 corresponds to 9; Subgroup B5 corresponds to 10; Subgroup Δ4 corresponds to 11; Subgroup Δ5 corresponds to 12; Subgroup ΓΓ4 corresponds to 5; Subgroup ΓΓ5 corresponds to 6; Subgroup AA4 corresponds to 7; Subgroup AA5 corresponds to 8; Subgroup ΔΔ4 corresponds to 13; Subgroup ΔΔ5 corresponds to 14; Subgroup BB4 corresponds to 15; Subgroup BB5 corresponds to 16.

### Example 23

### Effect of Biological Activity of Factor-Y

Effects of biological activity of Factor-Y were tested. Factor-Y is the addition of 10 ingredients: avocado oil+cuivre de sulfate+ester vit C+PABA+Cod liver oil+Vit A+Vit D+EPA+DHA+Agent-Y, Agent-Y is an ethanolic plant extract of the plant belonging to the genus Cinnamomum, the plant belonging to the genus of Jasminum and of green coffee bean Factor-Y does not affect basic cellular functions. More in particular, based on hormonal, enzymatic and biochemical analyzes factor-Y does not affect:
1. HORMONE-RECEPTORS INTERACTIONS: EGFa/b-EGFRa/b; CGH-CGHR; Melanocortin-MC 2/4ABR; Melanin concentrating hormone-MCHR; Melatonin-MTNR; KeraA/C-KeraR; ADPcD-ADPcDR; Histamine-HRH.
2. CELL CYCLE Cell cycle control Mitosis; Mitogen FRs.
3. APOPTOSIS: Apoptosis EC; Apoptosis KC; Apoptosis AC.
4. CELLULAR SENESCENE: OXID/Tang; p53 signaling pathway; Ubiquitin signaling.
5. CELL RESPONSE AND PROTECTION: MAPK signaling; ErbB signaling; Wnt signaling; Notch signaling; TGF-B signaling; VEGF signaling; JAK/STAT signaling; mTOR signaling; Endocytosis; Lysosome; Peroxisome; Regulation of autophagy.
6. CELLULAR ADHESION & ELASTISITY: Focal adhesion; Adherens junction; Tight junction; Gap junction; Regulation of actin/collagen/elastin; Cell adhesion molecules (CAMs)
7. MELANOGENESIS & KERATINIZATION: Melanogenesis; Keratin synthesis; Melanocytes control; Keratinocytes control.
8. WATER & ION CYCLE: Hydration Pro/Re control; Ion/CONC/control Ca/K/Na/PO-ABC transporters.
9. LIPID CYCLE: Membrane lipid control; Free lipid control; Adhesion lipid system
10. PROTEINS & AMINO ACIDS CYCLE: Protein export system; Protein FLD control; Amino acids bio-cycle.
11. COFACTORS & VITAMINS CYCLE: Cofactors cycle RiBo-X; Cofactors cycle Biotin-X; Vitamin cycle RRDB/Retinol.

### Example 25

### Effect of Biological Activity of Factor-Y

Factor-Y does not affect the cellular metabolism. In particular, based on hormonal, enzymatic and biochemical analyzes factor-Y does not affect:
1. HORMONE-RECEPTORS INTERACTIONS: Leptin-LEPR; Ghrelin-GSHR; Orexin-HCRTR; ADPc-ADPcR; Epinephrine-ADR; HCAD-HCADR; Motilin-MLNR; Calcitonin-CALCR; Thyrotropin releasing hormone-TRHR; Triidothyronine thyroxine-THR; Growth hormone-GHR; GHRHC-GHRHCR; GIP-GIPR; INS/GLY-INSR/GLYcR; Cortisol-NR3C1; Serotonin-SEROcR.
2. CARBONHYDRATE METABOLISM: Glycolysis/Gluconeogenesis; Citrate cycle (TCA cycle); Pentose phosphate pathway; Pentose and glucuronate interconversions; Fructose and mannose metabolism; Galactose metabolism; Ascorbate and aldarate metabolism; Starch and sucrose metabolism; Amino sugar and nucleotide sugar metabolism; Pyruvate metabolism; Glyoxylate and dicarboxylate metabolism; Propanoate metabolism; Butanoate metabolism; Inositol phosphate metabolism.
3. ENERGY METABOLISM: Oxidative phosphorylation; Methane metabolism; Nitrogen metabolism; Sulfur metabolism.
4. LIPID METABILISM: Fatty acid biosynthesis; Fatty acid elongation in mitochondria; Fatty acid metabolism; Synthesis and degradation of ketone bodies; Steroid biosynthesis; Primary bile acid biosynthesis; Steroid hormone biosynthesis; Glycerophospholipid metabolism; Ether lipid metabolism; Sphingolipid metabolism; Arachidonic acid metabolism; Linoleic acid metabolism; Alpha-Linolenic acid metabolism; Biosynthesis of unsaturated fatty acids.
5. NUCLEOTIDE METABOLISM: Purine metabolism; Pyrimidine metabolism
6. AMINO ACID METABOLISM: Alanine; aspartate and glutamate metabolism; Glycine, serine and threonine metabolism; Cysteine and methionine metabolism; Valine, leucine and isoleucine degradation; Valine, leucine and isoleucine biosynthesis; Lysine biosynthesis; Lysine degradation; Histidine metabolism; Tyrosine metabolism; Phenylalanine metabolism; Tryptophan metabolism; Phenylalanine, tyrosine and tryptophan biosynthesis.
7. METABOLISM OF OTHER AMINO ACIDS: Beta-Alanine metabolism; Taurine and hypotaurine metabolism; Selenoamino acid metabolism; Cyanoamino acid metabolism; D-Glutamine and D-glutamate metabolism; D-Arginine and D-ornithine metabolism; Glutathione metabolism.
8. METABOLISM & BIOSINTHESIS OF GLYCANS: N-Glycan biosynthesis; O-Glycan biosynthesis; O-Mannosyl glycan biosynthesis; Chondroitin sulfate biosynthesis; Heparan sulfate biosynthesis; Keratan sulfate biosynthesis; Glycosaminoglycan degradation; Glycosylphosphatidylinositol (GPI)-anchor biosynthesis; Glycosphingolipid biosynthesis-globo series; Glycosphingolipid biosynthesis-ganglio series; Other glycan degradation.
9. METABOLISM OF COFACTORS & VITAMINS: Thiamine metabolism; Riboflavin metabolism; Vitamin B6 metabolism; Nicotinate and nicotinamide metabolism; Pantothenate and CoA biosynthesis; Biotin metabolism; Lipoic acid metabolism; Folate biosynthesis; Retinol metabolism; Porphyrin and chlorophyll metabolism; Ubiquinone and other terpenoid-quinone biosynthesis.
10. SECONDARY METABOLITES BIOSYNTHESIS: Terpenoid backbone biosynthesis; Limonene and pinene degradation; Caffeine metabolism.

### Example 26

### Effect of Biological Activity of Factor-Y

In the solution of factor-Y is not detected the presence of microorganisms and biological toxins over the usual and acceptable limits. More in particular, based on molecular analysis, in the solution of the agent-Y are not detected:

1. MICROORGANISMS§: Acanthamoeba sp.; Acetobacter sp.; Acinetobacter baumannii; Actinomyces sp.; Actinomycetoma/Eumycetoma; Adenoviridae family; Aeromonas sp.; Agrobacterium sp.; Azorhizobium caulinodans; Azotobacter sp.; Anaplasma sp.; Ancylostoma sp.; Anisakis sp.; Arcanobacterium haemolyticum; Ascaris lumbricoides; Aspergillus genus; Astroviridae family; Babesia genus; Bacillus sp.; Bacteroides sp.; Balantidium sp.; Bartonella sp.; Baylisascaris genus; BK virus; Blastocystis hominis; Blastomyces dermatitidis; Bordetella sp.; Borrelia sp.; Brucella sp.; Bunyaviridae family; Burkholderia sp.; Caliciviridae family; Calymmatobacterium granulomatis; Campylobacter sp.; Candida sp.; Chlamydia sp.; Chlamydophila sp.; CJD prion; Clonorchis sinensis; Clostridium sp.; Coccidioides sp.; Colorado tick fever virus (CTFV); Corynebacterium sp.; Coxsackievirus Coxiella sp.; Crimean-Congo hemorrhagic fever virus; Cryptococcus neoformans; Cryptosporidium genus; Cyclospora sp.; Cytomegalovirus; Dengue viruses (DEN-1, DEN-2, DEN-3, DEN-4); Dientamoeba fragilis; Diphyllobothrium; Dracunculus medinensis; Ebolavirus (EBOV); Echinococcus genus; Ehrlichia sp.; Entamoeba histolytica; Enterobacter cloacae; Enterobius vermicularis; Enterococcus sp.; Enterovirus genus; Enteroviruses; Epidermophyton floccosum/Trichophyton-rubrum/Trichophyton mentagrophytes; Epstein-Ban Virus (EBV); Escherichia sp.; Eustrongylides sp.; Fasciola sp.; Fasciolopsis buski; FFI prion; Filarioidea superfamily; Flaviviruses; Fonsecaea pedrosoi; Francisella tularensis; Fusobacterium genus; Gardnerella vaginalis; Geotrichum candidum; Giardia sp.; Gnathostoma spinigerum; GSS prion; Guanarito virus; H1N1 virus; Haemophilus sp.; Halobaena caerulea; Helicobacter pylori; Hepatitis A Virus; Hepatitis B Virus; Hepatitis C Virus; Hepatitis D Virus; Hepatitis E Virus; Herpes simplex virus 1/2 (HSV-1/HSV-2); Histoplasma capsulatum HIV (Human immunodeficiency virus); Hortaea werneckii; Human bocavirus (HBoV); Human cytomegalovirus; Human herpesvirus 6 (HHV-6); Human herpesvirus 7 (HHV-7)/+8; Human metapneumovirus (hMPV); Human papillomavirus (HPV); Human parainfluenza viruses (HPIV); Hymenolepis sp.; Isospora belli; Influenza virus; JC virus; Junin virus; Kingella sp.; Klebsiella sp.; Kuru prion; Lactobacillus sp.; Lactococcus lactis; Lassa virus; Legionella pneumophila; Leishmania genus; Leptospira genus; Listeria sp.; Lymphocytic choriomeningitis virus (LCMV); Machupo virus; Malassezia genus; Marburg virus; Measles virus; Metagonimus yokagawai; Methanobacterium sp.; Microbacterium sp.; Micrococcus luteus; Microsporidia phylum; Miscellaneous sp.; Molluscum contagiosum virus (MCV); Moraxella catarrhalis; Mucorales order (Mucormycosis)/Entomophthorales; Mumps virus; Mycobacterium sp.; Mycoplasma sp.; Naegleria fowleri Nanophyetus sp.; Neisseria sp.; Nocardia sp.; Norovirus; Onchocerca volvulus; Orthomyxoviridae family; Pachyptila sp./itm.; Papillomavirus; Paracoccidioides brasiliensis; Paragonimus westermani/Paragonimus species; Parainfluenza virus; Parvovirus; B19 Pasteurella sp.; Pediculus humanus capitis; Peptostreptococcus; Phthirus pubis; Piedraia hortae; Plasmodium genus; Plesiomonas shigelloides; Pneumocystis jirovecii; Poliovirus; Porphyromonas gingivalis; Prevotella genus; Pseudomonas aeruginosa; Rabies virus; Respiratory syncytial virus (RSV); Rhizobium radiobacter; Rhinosporidium seeberi; Rhinoviruses/Coronaviruses; Rickettsia sp.; Rift Valley fever virus; Rochalimaea sp.; Rotavirus; Rothia dentocariosa; Rubella virus; Sabia Salmonella sp.; Sarcocystis hominis; Sarcoptes scabiei; SARS coronavirus; Schistosoma genus; Serratia marcescens; Shigella sp.; Sin Nombre virus; Sporothrix schenckii; Staphylococcus sp.; Stenotrophomonas maltophilia; Streptobacillus moniliformis; Streptococcus sp.; Strongyloides stercoralis; Taenia genus; Taenia solium; Toxocara canis/Toxocara cati; Toxoplasma sp.; Treponema sp.; Trichinella sp.; Trichomonas vaginalis; Trichophyton genus; Trichosporon beigelii; Trichuris trichiura; Trypanosoma sp.; Ureaplasma urealyticum; Varicella zoster virus (VZV); Variola major/Variola minor vCJD prion; Venezuelan equine encephalitis virus Vibrio sp.; West Nile virus; Wolbachia Wuchereria sp.; Yellow fever virus; Yersinia sp.
2. BIOLOGICAL TOXINS: 3-Nitropropionic acid; Aflatoxins; Alkaloids; Bacillus cereus; Ciguatera poisoning; Citreoviridin; Clostridium botulinum; Clostridium perfringens; Cyclopiazonic acid; Cytochalasins; Ergopeptine alkaloids; Ergot alkaloids; Ergotamine; Foxglove; Fumonisins; Fusaric acid; Fusarochromanone; Gempylotoxin; Grayanotoxin; Kojic acid; Lolitrem alkaloids; Moniliformin; Mushroom toxins; Nivalenol; Ochratoxins; Oosporeine; Patulin; Phomopsins; Phytohaemagglutinin; Poisonous hemlock; Pyrrolizidine alkaloids; Scombrotoxin; Shellfish toxin; Sporidesmin A; Staphylococcus aureus; Sterigmatocystin; Tetrodotoxin; Tremorgenic mycotoxins; Trichothecenes; Zearalenols.

### Example 27

### Effect of Biological Activity of Factor-Y

In particular, it was proved that in the solution of factor-Y are not detected GMOs. More in particular, based on molecular analysis, in the solution of factor-Y are not detected:

GMO FRAMES; CaMVp35S; T-nos sp/ITM; Bt11; Bt176; Bt4332/pQ4511; CMVprom012; MON810plus; QP35S SP/ITMs; cDNAmutR112; cDNAmutF112; cENHCmutR122; cENHCmutF122; cORImutR1131; cORImutF1131; cAUGmutTER/R/F0542; ELcdpT4; ELcd122/77; ELrASIA/09; ELrAFRC/09; ELrEE/010; ELrUS/010; EDNA/09; CcDNApbrWQ11; qR/Fori; pR/Fter.

### Example 28

### Effect of Biological Activity of Factor-Y

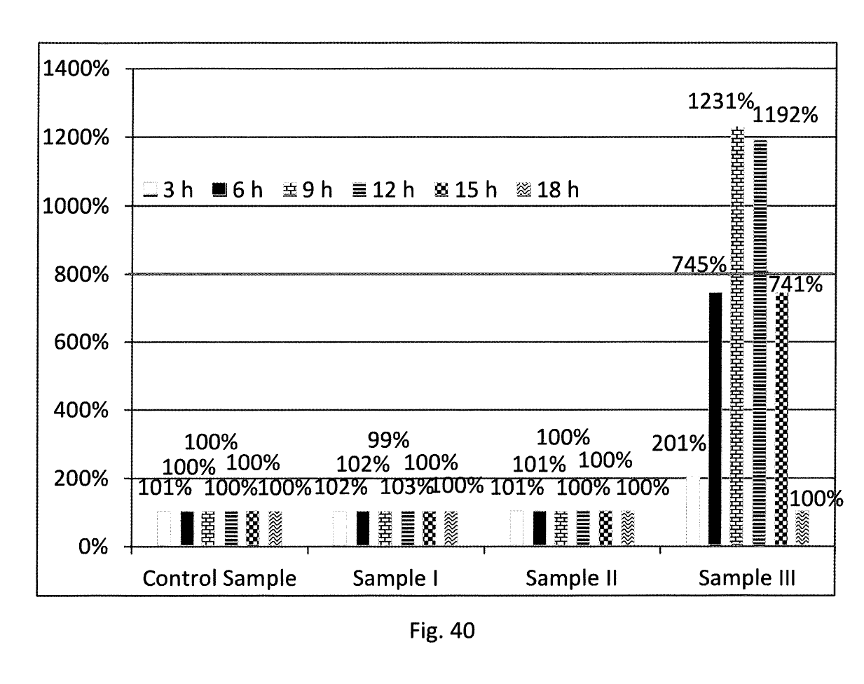

It was confirmed that factor-Y activates the transcription factor NF-kB by 12.3 times. Increasing time of cell exposure to the solution of the agent-Y leads to increase the activation of the transcription factor NF-kB by 12.3 times (1231%) with a maximum effect at 9 to 12 hours in epithelial cells and B-cells. FIG. 40 shows Percentage % activation of the transcription factor NF-kB (p<0.001). In particular in the experiments leading to the results of FIG. 40,
1) basic components of the solution of factor-Y are: ester vitC, PABA, cod liver oil, vitA, vitD, EPA+DHA;
2) liquids are: avocado oil and cuivre de sulfate and
3) agent-Y: is the 3 plant ethanolic extract.

SAMPLE I: contains the basic components of the solution of factor-Y, without the liquids and agent-Y; SAMPLE II: contains the basic components of the solution of factor-Y, the liquids, without agent-Y; SAMPLE III: contains the basic components of the solution of factor-Y the liquids and agent-Y.

Increasing of the transcription factor NF-kB activation up to 12.3 times with increasing time of exposure of the cells to the solution of the factor-Y with maximum effect from 9 to 12 hours is only achieved by the synergy of agents in agent solution-Y (sample III).

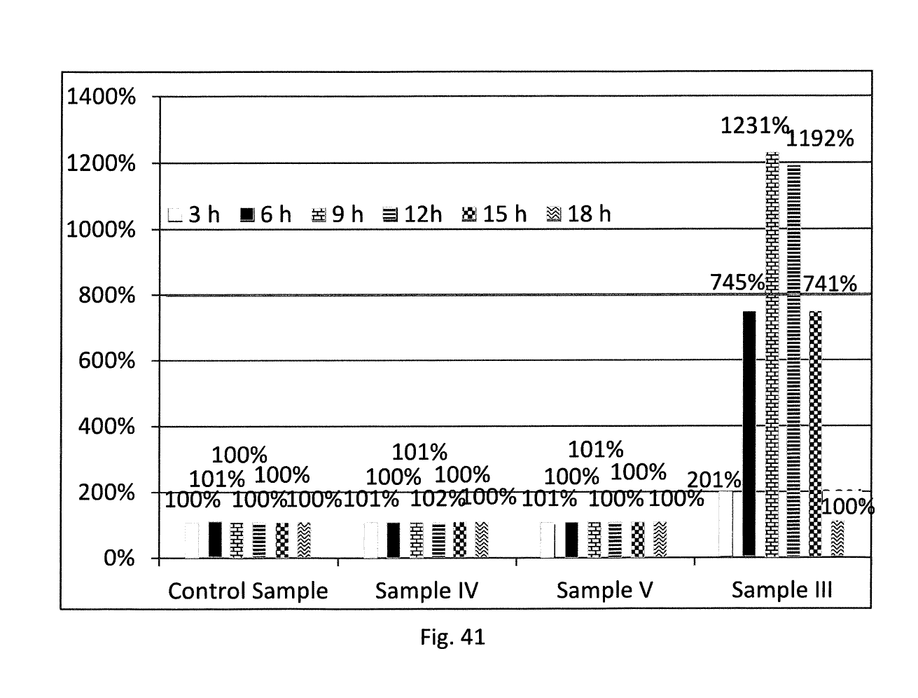

FIG. 41 shows Percentage % activation of the transcription factor NF-kB (p<0.001), In particular in the illustration of FIG. 41
1) the asic components of the solution of factor-Y are: ester vitC, PABA, cod liver oil, vitA, vitD, EPA+DHA;
2) liquids are: avocado oil and cuivre de sulfate and
3) agent-Y: is the 3 plant ethanolic extract.

SAMPLE IV: contains no basic constituents of the solution of factor-Y and no agent-Y, ONLY the liquids; SAMPLE V: contains only the agent-Y without the basic constituents of the solution of factor-Y, without the liquids.

### Example 29

### Effect of Biological Activity of Factor-Y

It was proved that factor-Y blocks the binding of protein TRAF6 to signal transduction pathway that begins from the permanently activated CD40 receptor. In particular, permanently activated receptor CD40 is in epithelial cells and B-lymphocytes mimics pathological autoimmune diseases.

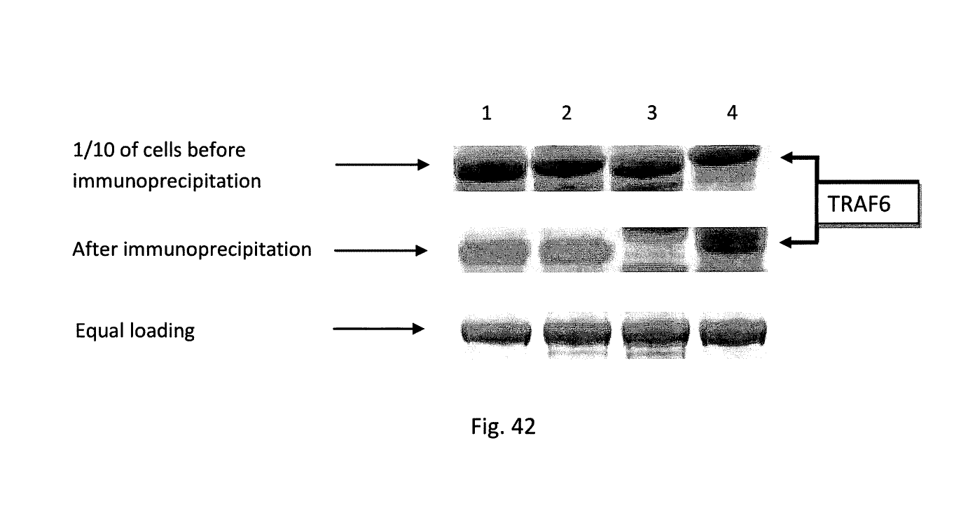

FIG. 42 shows Immunoprecipitation of the complex based TRAFsfml-TRAF6 induced by activated cytoplasmic domain of the receptor CD40. With reference to FIG. 42, reference number 1 indicates “Sample I”, reference number 2 indicates “Sample II”, Reference number 3 indicates “Sample III”, Reference number 4 indicates “Control Sample”, wherein
1) the basic components of the solution of factor-Y are: ester vitC, PABA, cod liver oil, vitA, vitD, EPA+DHA;
2) liquids are: avocado oil and cuivre de sulfate and
3) agent-Y: is the 3 plant ethanolic extract.
   SAMPLE I: contains the basic components of the solution of factor-Y, without the liquids and agent-Y; SAMPLE II: contains the basic components of the solution of factor-Y, the liquids, without agent-Y; SAMPLE III: contains the basic components of the solution of factor-Y the liquids and agent-Y.

It appears that this phenomenon is achieved only by the synergy of agents of the solution of factor-Y (Sample III).

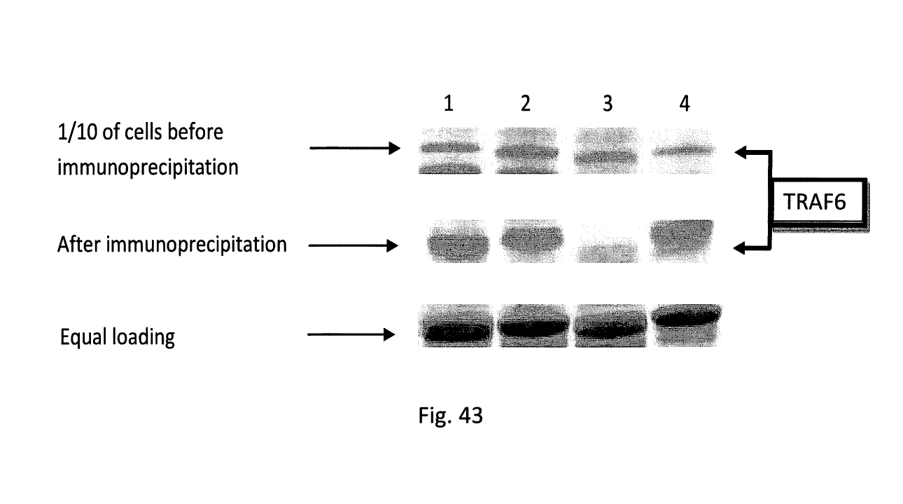

FIG. 43 shows Immunoprecipitation of the complex based TRAFsfml-TRAF6 induced by activated cytoplasmic domain of the CD40 receptor.

With reference to FIG. 42, reference number 1 indicates “Sample IV”, reference number 2 indicates “Sample V”, reference number 3 indicates “Sample III”, reference number 4 indicates “Control Sample”.
1) basic components of the solution of factor-Y are: ester vitC, PABA, cod liver oil, vitA, vitD, EPA+DHA;
2) liquids are: avocado oil and cuivre de sulfate and
3) agent-Y: is the 3 plant ethanolic extract.
   SAMPLE IV: contains no basic constituents of the solution of factor-Y and no agent-Y, only the liquids; SAMPLE V: contains only the agent-Y without the basic constituents of the solution of factor-Y, without the liquids.

### Example 30

### Effect of Biological Activity of Factor-Y

It was proved that Factor-Y does not affect the normal growth and health of wild type mice.

To study such effect of factor-Y in mice, were organized the following groups of animals.

20 wild type mice (wt C57/C57) 3 months old were divided into two groups of 10 mice each. The first group named A and received along with the food the solution of the factor-Y every 12 hours per day for three weeks while the second group B received placebo solution likewise with food daily every 12 hours for three weeks. After the end of treatment showed no difference in the growth and health of the mice of both groups A and B. Specifically mice were studied for the tests indicated in Table 17 here below.

TABLE 17

| | Group A | Group B |
|---|---|---|
| Weight | Normal | Normal |
| complete blood tests | Normal | Normal |
| basic biochemical analyzes | Normal | Normal |
| examination by veterinary | Normal | Normal |
| specialists in basic | | |
| physiological functions | | |

### Example 31

### Effect of Biological Activity of Factor-Y

Factor-Y eliminates the presence of autoantibodies against DNA of the thymus of transgenic mice LMP1/CD40. To study such effect of factor-Y in mice, were organized the following groups of animals.

20 LMP1/CD40 transgenic mice 3 months old were divided into two groups of 10 mice each. The first group named A and received along with the food the solution of the factor-Y every 12 hours per day for three weeks while the second group B received placebo solution likewise with food daily every 12 hours for three weeks. In serum of the animals detect antibodies against thymus DNA by the method of ELISA and found that the transgenic mice of group B LMP1/CD40 have autoantibodies against thymus DNA in serum at much higher concentration than the mice of Group A.

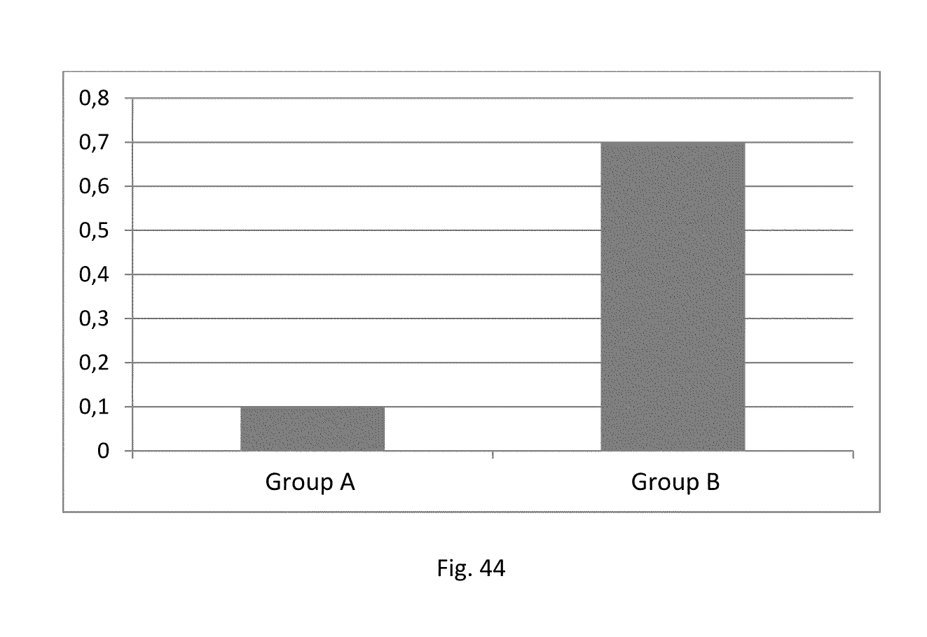

FIG. 44 shows Concentration of antibodies against thymus DNA (OD) in Group A and Group B. Detection of antibodies against thymus DNA by the method of ELISA in mice sera. The statistical analysis was performed using Student control method and specifically by applying two-tailed unpaired Student t test (p<0.001).

### Example 32

### Effect of Biological Activity of Factor-Y

Factor-Y eliminates the deposition of immunoglobulin IgG in the kidney of transgenic mice LMP1/CD40. To study such effect of factor-Y in mice, were organized the following groups of animals.

20 LMP1/CD40 and transgenic mice 3 months old were divided into two groups of 10 mice each. The first group named A and received along with the food the solution of the factor-Y every 12 hours per day for three weeks while the second group B received placebo solution likewise with food daily every 12 hours for three weeks. A fuller and more convincing observation of eliminating the existence of autoantibodies in transgenic mice LMP1/CD40 is the detection of immunoglobulins IgG deposits in the kidneys of mice. Specifically from mice were isolated kidneys and made incisions and detection of immunoglobulins IgG mouse with fluorescence. The sections revealed the presence of the immunoglobulins IgG deposits in the kidney of transgenic mice LMP1/CD40 group B, which are absent from the sections of the transgenic mice of Group A.

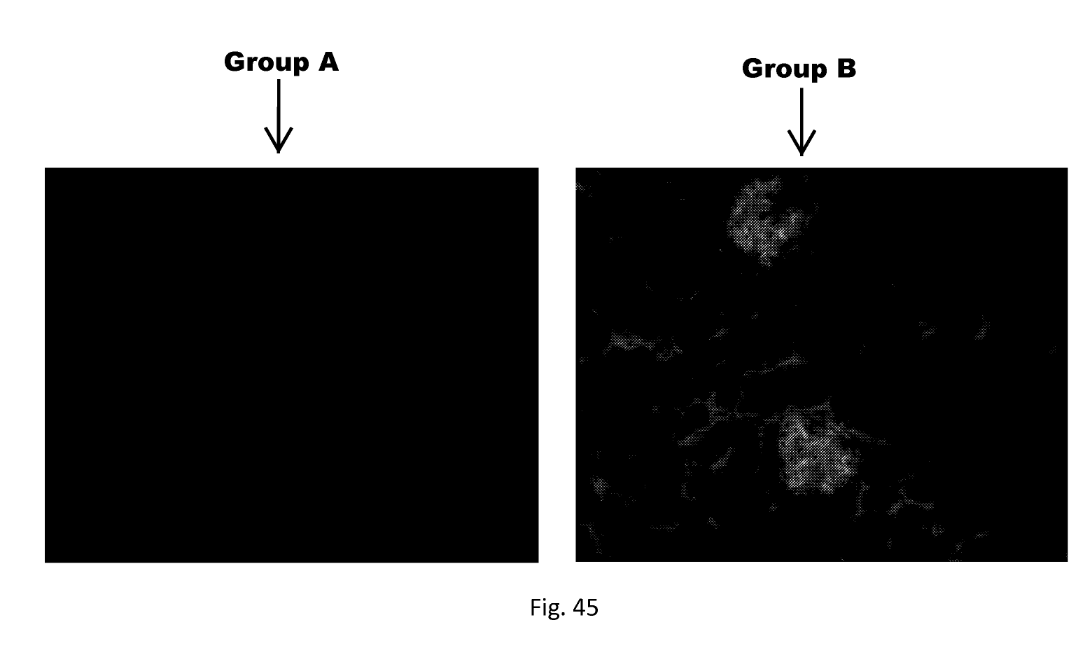

FIG. 45 shows Kidney sections and detection of immunoglobulins IgG mouse with fluorescence of Group A and Group B.

### Example 33

### Effect of Biological Activity of Factor-Y

Factor-Y eliminates perivascular inflammation of the liver of transgenic mice LMP1/CD40. To study such effect of factor-Y in mice, were organized the following groups of animals.

20 LMP1/CD40 and transgenic mice 3 months old were divided into two groups of 10 mice each. The first group named A and received along with the food the solution of the factor-Y every 12 hours per day for three weeks while the second group B received placebo solution likewise with food daily every 12 hours for three weeks. Liver sections from transgenic mice LMP1/CD40 incubated with hematoxylin-eosin and observed that in sections of the transgenic mice of group A are not observed perivascular inflammation, which are particularly common in sections of the transgenic mice of group B.

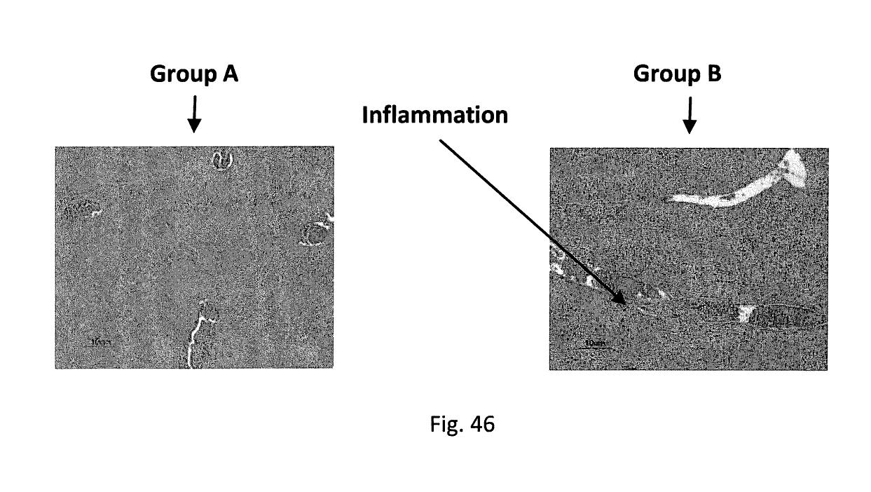

FIG. 46 shows Sections of liver of transgenic mice LMP1/CD40 hematoxylin-eosin staining in Group A and Group B.

### Example 34

### Effect of Biological Activity of Factor-Y

Factor-Y does not eliminate all the characteristics of abnormal functioning of the immune system of transgenic mice LMP1/CD40. To study such effect of factor-Y in mice, were organized the following groups of animals.

20 LMP1/CD40 and transgenic mice 3 months old were divided into two groups of 10 mice each. The first group named A and received along with the food the solution of the factor-Y every 12 hours per day for three weeks while the second group B received placebo solution likewise with food daily every 12 hours for three weeks. The solution of the factor-Y does not eliminate all the characteristics of abnormal functioning of the immune system of transgenic mice LMP1/CD40. The results for Group A and Group B are summarized in Table 18 here below.

TABLE 18

| Group A | Group B |
|---|---|
| Elevated levels of macrophage | Elevated levels of macrophage |
| Elevated levels of polymorphonuclear neutrophils | Elevated levels of polymorphonuclear neutrophils |
| Elevated levels of CD4 + T-lymphocyte | Elevated levels of CD4 + T-lymphocyte |
| Elevated levels of CD8 + T-lymphocytes | Elevated levels of CD8 + T-lymphocytes |
| Elevated levels of CD69 | Elevated levels of CD69 |
| Elevated levels of CD80 | Elevated levels of CD80 |
| Elevated levels of CD86 | Elevated levels of CD86 |
| Elevated levels of non-specific IgM | Elevated levels of non-specific IgM |
| Reduced levels of specific antibodies against | Reduced levels of specific antibodies against |
| pathogen | pathogen |
| Suppression forming ability of germinal centers | Suppression forming ability of germinal centers |
| Reduced affinity antibodies specific for the | Reduced affinity antibodies specific for the |
| immunogen lowered affinity | immunogen lowered affinity antibodies |

### Example 35

### Effect of Biological Activity of Factor-Y

In vivo study of the effects of factor-Y in mouse models simulating Alzheimer's disease. To study the effect of factor-Y in mice, were organized the following groups of animals.

five mice from each mouse model and five wild type mice treated daily every 12 hours by food with factor-Y for three months. Similarly, five mice from each model along with five wild type mice treated daily every 12 hours by food with the placebo solution for three months.

The study of phenotypic characteristics of mice showed no variation with the impact of factor-Y. The behavioral memory tests, such as timed detection of the exit on a maze showed no statistically significant differences between groups.

It was confirmed that Factor-Y increases the biological activity of cholinergic synapses in all mouse models for Alzheimer's disease, as indicated in Table 19 here below.

TABLE 19

| Model | Metabolic Procedure | Biological activity | Biological reactivity |
|---|---|---|---|
| 1 | Cholinergic synapse-Mus musculus (mouse) | 142% | 100% |
| 2 | Cholinergic synapse-Mus musculus (mouse) | 143% | 100% |
| 3 | Cholinergic synapse-Mus musculus (mouse) | 140% | 100% |
| 4 | Cholinergic synapse-Mus musculus (mouse) | 142% | 100% |
| 5 | Cholinergic synapse-Mus musculus (mouse) | 141% | 100% |
| 6 | Cholinergic synapse-Mus musculus (mouse) | 141% | 100% |
| 7 | Cholinergic synapse-Mus musculus (mouse) | 142% | 100% |
| 8 | Cholinergic synapse-Mus musculus (mouse) | 144% | 100% |
| 9 | Cholinergic synapse-Mus musculus (mouse) | 142% | 100% |
| 10 | Cholinergic synapse-Mus musculus (mouse) | 141% | 100% |
| 11 | Cholinergic synapse-Mus musculus (mouse) | 141% | 100% |
| 12 | Cholinergic synapse-Mus musculus (mouse) | 142% | 100% |
| 13 | Cholinergic synapse-Mus musculus (mouse) | 141% | 100% |
| 14 | Cholinergic synapse-Mus musculus (mouse) | 141% | 100% |
| 15 | Cholinergic synapse-Mus musculus (mouse) | 141% | 100% |
| 16 | Cholinergic synapse-Mus musculus (mouse) | 142% | 100% |
| 17 | Cholinergic synapse-Mus musculus (mouse) | 140% | 100% |
| 18 | Cholinergic synapse-Mus musculus (mouse) | 141% | 100% |
| 19 | Cholinergic synapse-Mus musculus (mouse) | 143% | 100% |
| 20 | Cholinergic synapse-Mus musculus (mouse) | 141% | 100% |
| 21 | Cholinergic synapse-Mus musculus (mouse) | 142% | 100% |
| 22 | Cholinergic synapse-Mus musculus (mouse) | 141% | 100% |
| 23 | Cholinergic synapse-Mus musculus (mouse) | 142% | 100% |
| 24 | Cholinergic synapse-Mus musculus (mouse) | 142% | 100% |

It was then confirmed that Factor-Y increases the expression of NGF (Nerve Growth Factor) in all mouse models for Alzheimer's disease, as indicated in Table 20 here below.

TABLE 20

| | | Three months | Three months |
|---|---|---|---|
| | | treatment | treatment |
| Model | Metabolic Procedure | with factor-Y | with placebo control |
| 1 | Nerve Growth Factor exp-Mus musculus (mouse) | 174% | 100% |
| 2 | Nerve Growth Factor exp-Mus musculus (mouse) | 174% | 100% |
| 3 | Nerve Growth Factor exp-Mus musculus (mouse) | 175% | 100% |
| 4 | Nerve Growth Factor exp-Mus musculus (mouse) | 174% | 100% |
| 5 | Nerve Growth Factor exp-Mus musculus (mouse) | 175% | 100% |
| 6 | Nerve Growth Factor exp-Mus musculus (mouse) | 175% | 100% |
| 7 | Nerve Growth Factor exp-Mus musculus (mouse) | 174% | 100% |
| 8 | Nerve Growth Factor exp-Mus musculus (mouse) | 175% | 100% |
| 9 | Nerve Growth Factor exp-Mus musculus (mouse) | 175% | 100% |
| 10 | Nerve Growth Factor exp-Mus musculus (mouse) | 174% | 100% |
| 11 | Nerve Growth Factor exp-Mus musculus (mouse) | 175% | 100% |
| 12 | Nerve Growth Factor exp-Mus musculus (mouse) | 175% | 100% |
| 13 | Nerve Growth Factor exp-Mus musculus (mouse) | 174% | 100% |
| 14 | Nerve Growth Factor exp-Mus musculus (mouse) | 175% | 100% |
| 15 | Nerve Growth Factor exp-Mus musculus (mouse) | 176% | 100% |
| 16 | Nerve Growth Factor exp-Mus musculus (mouse) | 174% | 100% |
| 17 | Nerve Growth Factor exp-Mus musculus (mouse) | 175% | 100% |
| 18 | Nerve Growth Factor exp-Mus musculus (mouse) | 175% | 100% |
| 19 | Nerve Growth Factor exp-Mus musculus (mouse) | 175% | 100% |
| 20 | Nerve Growth Factor exp-Mus musculus (mouse) | 175% | 100% |
| 21 | Nerve Growth Factor exp-Mus musculus (mouse) | 175% | 100% |
| 22 | Nerve Growth Factor exp-Mus musculus (mouse) | 174% | 100% |
| 23 | Nerve Growth Factor exp-Mus musculus (mouse) | 175% | 100% |
| 24 | Nerve Growth Factor exp-Mus musculus (mouse) | 175% | 100% |

It was then confirmed that Factor-Y decreases the phosphorylation of Tau protein in all mouse models for Alzheimer's disease, as indicated in Table 21 here below.

TABLE 21

| | | Three | Three |
|---|---|---|---|
| | | months treatment | months treatment |
| Model | Metabolic Procedure | with factor-Y | with placebo control |
| 1 | Tau Protein Phlt-Mus musculus (mouse) | 71% | 100% |
| 2 | Tau Protein Phlt-Mus musculus (mouse) | 71% | 100% |
| 3 | Tau Protein Phlt-Mus musculus (mouse) | 73% | 100% |
| 4 | Tau Protein Phlt-Mus musculus (mouse) | 74% | 100% |
| 5 | Tau Protein Phlt-Mus musculus (mouse) | 72% | 100% |
| 6 | Tau Protein Phlt-Mus musculus (mouse) | 71% | 100% |
| 7 | Tau Protein Phlt-Mus musculus (mouse) | 74% | 100% |
| 8 | Tau Protein Phlt-Mus musculus (mouse) | 71% | 100% |
| 9 | Tau Protein Phlt-Mus musculus (mouse) | 71% | 100% |
| 10 | Tau Protein Phlt-Mus musculus (mouse) | 74% | 100% |
| 11 | Tau Protein Phlt-Mus musculus (mouse) | 73% | 100% |
| 12 | Tau Protein Phlt-Mus musculus (mouse) | 72% | 100% |
| 13 | Tau Protein Phlt-Mus musculus (mouse) | 74% | 100% |
| 14 | Tau Protein Phlt-Mus musculus (mouse) | 73% | 100% |
| 15 | Tau Protein Phlt-Mus musculus (mouse) | 71% | 100% |
| 16 | Tau Protein Phlt-Mus musculus (mouse) | 74% | 100% |
| 17 | Tau Protein Phlt-Mus musculus (mouse) | 71% | 100% |
| 18 | Tau Protein Phlt-Mus musculus (mouse) | 71% | 100% |
| 19 | Tau Protein Phlt-Mus musculus (mouse) | 72% | 100% |
| 20 | Tau Protein Phlt-Mus musculus (mouse) | 72% | 100% |
| 21 | Tau Protein Phlt-Mus musculus (mouse) | 72% | 100% |
| 22 | Tau Protein Phlt-Mus musculus (mouse) | 71% | 100% |
| 23 | Tau Protein Phlt-Mus musculus (mouse) | 73% | 100% |
| 24 | Tau Protein Phlt-Mus musculus (mouse) | 71% | 100% |

### Example 36

### Effect of Biological Activity of Factor-Y

In vivo study of the effects of the factor-Y in mouse models that simulate depression. To study such effect of factor-Y in mice, were organized the following groups of animals.

five mice from each mouse model and five wild type mice treated daily every 12 hours by food with factor-Y for five weeks. Similarly, five mice from each model along with five wild type mice treated daily every 12 hours by food with the placebo solution for five weeks.

The study of phenotypic characteristics of mice showed no variation with the impact of factor-Y. The behavioral tests such as forced swimming test (FST) and tail suspension test (TST) showed no statistically significant differences between groups.

### Example 37

### Effect of Biological Activity of Factor-Y

Factor-Y increases the biological activity of serotonergic synapses in mouse models that simulate depression, as indicated in Table 22 here below.

TABLE 22

| | Three months | Biological | Biological |
|---|---|---|---|
| Metabolic Procedure | treatment | activity | reactivity |
| Serotonergic synapse- | Factor-Y | 162% | 100% |
| Mus musculus (mouse) | | | |
| Serotonergic synapse- | Placebo control | 100% | 100% |
| Mus musculus (mouse) | | | |

### Example 38

### Effect of Biological Activity of Factor-Y

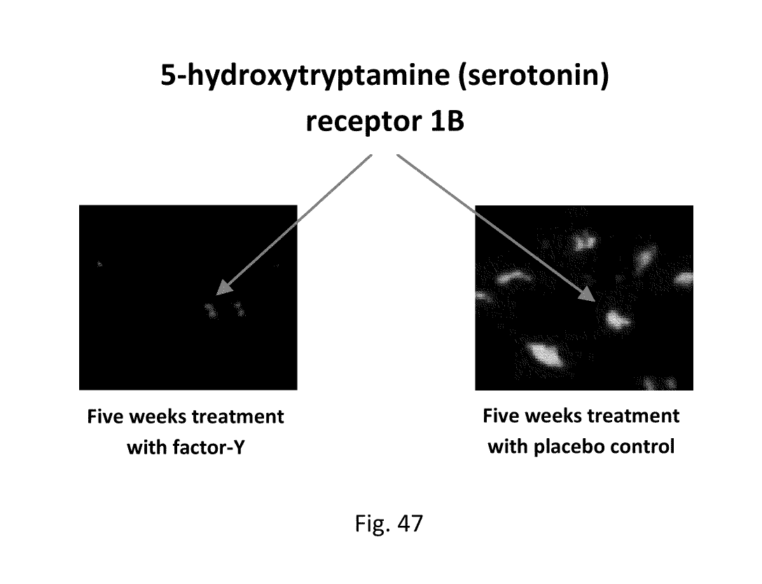

Factor-Y reduce gene expression of Htr1b gene (receptor 5-hydroxytryptamine or serotonin receptor 1B) in mouse models that simulate depression. Such effect is confirmed in FIG. 47, which shows Kidney sections and detection of immunoglobulins IgG mouse with fluorescence.

The examples set forth above are provided to give those of ordinary skill in the art a complete disclosure and description of how to make and use the embodiments of the catalyst, and related, compositions, methods and systems of the disclosure, and are not intended to limit the scope of what the Applicants regard as their disclosure. Modifications of the above-described modes for carrying out the disclosure can be used by persons of skill in the art, and are intended to be within the scope of the following claims.

The entire disclosure of each document cited (including patents, patent applications, journal articles including related supplemental and/or supporting information sections, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations, which is not specifically disclosed herein.

Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations, which is not specifically disclosed herein.

“Optional” or “optionally” means that the subsequently described circumstance may or may not occur, so that the description includes instances where the circumstance occurs and instances where it does not according to the guidance provided in the present disclosure. For example, the phrase “optionally substituted” means that a non-hydrogen substituent may or may not be present on a given atom, and, thus, the description includes structures wherein a non-hydrogen substituent is present and structures wherein a non-hydrogen substituent is not present. It will be appreciated that the phrase “optionally substituted” is used interchangeably with the phrase “substituted or unsubstituted.” Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned can be identified in view of the desired features of the compound in view of the present disclosure, and in view of the features that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

1. An ethanolic plant extract obtainable by contacting aerial parts of at least a first plant and a second plant with ethanol with a volume/volume ratio first plant:ethanol from about 1:1.25 to about 1:1.75 and a volume/volume ratio second plant:ethanol from about 1:10 to about 1:18 wherein the first plant belongs to plant genus Jasminum and is in flowering stage, and the second plant belongs to plant genus Cinnamomum.

2. The ethanolic plant extract of claim 1, wherein the volume/volume ratio first plant:ethanol ratio is about 1:1.5 and the volume/volume ratio second plant:ethanol ratio is about 1:14

3. The ethanolic plant extract of claim 1, wherein the first plant is Jasminum officinale

4. The ethanolic plant extract of claim 1, wherein the second plant is Cinnamomum verum

5. The ethanolic plant extract of claim 1 wherein the extract comprises alkaloids, phenolic acids and derivatives, polyphenols, terpenes and steroids, methylated phenols, and benzopyrans, carbohydrates free fatty acids and triglycerides.

6. The ethanolic plant extract of claim 1, wherein the aerial parts of at least a first and a second plants further comprise aerial parts of a third plant, the third plant belongs to the genus Coffea and the aerial parts are green beans of the third plant, and wherein the contacting further comprises contacting the aerial parts of the third plants with ethanol with a volume/volume ratio third plant:ethanol from about 1:25 to about 1:35.

7. The ethanolic plant extract of claim 6, wherein the volume/volume ratio third plant:ethanol ratio is about 1:30

8. The ethanolic plant extract of claim 6, wherein the extract comprises alkaloids, phenolic acids and derivatives, polyphenols, terpenes and steroids, methylated phenols, and benzopyrans, carbohydrates free fatty acids and triglycerides.

9. An ethanolic plant extract comprising a mixture of alkaloids, phenolic acids and derivatives thereof, polyphenols, terpenes, steroids, methylated phenols; benzopyrans; carbohydrates; free fatty acids and triglycerides, the mixture obtained by performing ethanol extraction of aerial parts of at least a first plant and a second plant, wherein the first plant belongs to plant genus Jasminum, and the second plant belongs to plant genus Cinnamomum

10. The ethanolic plant extract of claim 9, wherein the extraction is performed by contacting aerial parts of at least a first plant and a second plant with ethanol at a temperature comprised between about 15° C. and about 35° C. for a time comprised between 7 days and 21 days.

11. The ethanolic plant extract of claim 9, wherein the aerial parts of at least a first and a second plants further comprise aerial parts of a third plant, the third plant belongs to the genus Coffea and the aerial parts are green beans of the third plant,

12. The ethanolic plant extract of claim 11, wherein the extraction is performed by contacting aerial parts of the first plant the a second plant and the third plant with ethanol at a temperature comprised between about 15° C. and about 35° C. for a time comprised between 11 days and 17 days.

13. The ethanolic plant extract of claim 11, wherein the alkaloids comprise caffeine.

14. The ethanolic plant extract of claim 11, wherein the phenolic acids and derivatives are selected from the group consisting of cinnamic aldehyde, trans cinnamic acid, 3, 4-dihydroxy-hydrocinnamic acid, chlorogenic acid or a combination thereof.

15. The ethanolic plant extract of claim 11, wherein the polyphenols are selected from the group consisting of flavonoids, lignans or a combination thereof.

16. The ethanolic plant extract of claim 11, wherein the carbohydrates are selected from the group consisting of sucrose, glucose or a combination thereof.

17. The ethanolic plant extract of claim 11, wherein the steroids are selected from the group consisting of limonene, α-copaene, β-sitosterol or a combination thereof.

18. The ethanolic plant extract of claim 11, wherein the methylated phenols comprise tocopherols.

19. The ethanolic plant extract of claim 11, wherein the benzopyrans comprise coumarin.

20. The ethanolic plant extract of claim 9, wherein the first plant is Jasminum officinale

21. The ethanolic plant extract of claim 9, wherein the second plant is Cinnamomum verum

22. A formulation comprising one or more ethanolic plant extracts according to claim 1 or 9, and at least one additional active agent selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid (DHA) or a combination thereof.

23. The formulation of claim 22, wherein the ethanol extract is comprised in an amount of 0, 25-0, 05 ml.

24. The formulation of claim 22, wherein the avocado oil is comprised in an amount of 0, 1-0.025 ml.

25. The formulation of claim 22, wherein the copper sulfate is comprised in an amount of 1.2-0.4 mg

26. The formulation of claim 22, wherein the ester of Vitamin C is comprised in an amount of 400-100 mg

27. The formulation of claim 22 wherein the para amino benzoic acid is comprised in an amount of 400-100 mg.

28. The formulation of claim 22, wherein the cod liver oil is comprised in an amount of 400-100 mg

29. The formulation of any claim 22, wherein the vitamin A is comprised in an amount of 600-200 gRE

30. The formulation of claim 22, wherein the vitamin D is comprised in an amount of 2-0.5μg

31. The formulation of claim 22, wherein the Eicosapentaenoic acid (EPA) is comprised in an amount of 30-10 mg.

32. The formulation of claim 22, wherein the Docosahexaenoic acid (DHA) is comprised in an amount of 30-10 mg.

33. The formulation of claim 22, including 0.15 ml±10% or 3 drops±15% of the ethanolic plant extract, together with 0.05 ml±10% of avocado oil, 0.83 mg±10% of copper sulfate, 250 mg±10% of ester vitC, 275 mg±10% of Para amino benzoic acid, 250 mg±10% of Cod liver oil, 400gRE±10% of vitA, 1, 25 μg±10% of vitD, 22 mg±10% of EPA and/or 19 mg±10% of DHA.

34. A method to elicit a biological response in a biological environment, the method comprising
contacting the biological environment with a formulation of claim 22, in an effective amount to increase activation of transcription factor NF-kB, increase expression of NGF (Nerve Growth Factor); increasing the biological activity of cholinergic synapses; and/or reduce gene expression of Htr1b gene (receptor 5-hydroxytryptamine or serotonin receptor 1B),
wherein the formulation comprises avocado oil, copper sulfate, ester-vitamin C, para aminobenzoic acid; cod liver oil, vitamin A, vitamin D, Eicosapentaenoic acid (EPA) and Docosahexaenoic acid (DHA), agent-X.

35. A method to treat an individual, the method comprising
administering to the individual an amount of the formulation of claim 22 effective to increase activation of transcription factor NF-kB, increase expression of NGF (Nerve Growth Factor); increasing the biological activity of cholinergic synapses; decreasing the phosphorylation of Tau protein; and/or reduce gene expression of Htr1b gene (receptor 5-hydroxytryptamine or serotonin receptor 1B).

36. The method of claim 35, wherein the administering is performed in an amount effective to increase activation of Nf-kB by 12.3 times (1231%) with respect to a baseline.

37. The method of claim 36, wherein the administering is performed in an amount effective to increase activation of Nf-kB with a maximum effect at 9 to 12 hours in epithelial cells and B-cells.

38. A method for providing a plant extract capable of eliciting a biological effect in an individual, the method comprises
contacting an aerial part of at least a first plant and a second plant with ethanol with a molar ratio first plant:ethanol from about 1:1.2.5 to about 1:1:75 and a molar ratio second plant:ethanol from about 1:10 to about 1:18 wherein the first plant belongs to plant genus Jasminum and is in flowering stage, and the second plant belongs to plant genus Cinnamomum.

39. A method to treat an individual, the method comprising
administering to the individual an amount of the formulation a formulation of claim 22, in an effective amount to increase activation of transcription factor NF-kB, increase expression of NGF (Nerve Growth Factor); increasing the biological activity of cholinergic synapses; decreasing the phosphorylation of Tau protein; and/or reduce gene expression of Htr1b gene (receptor 5-hydroxytryptamine or serotonin receptor 1B),
wherein the formulation comprises avocado oil, copper sulfate, ester-vitamin C, para aminobenzoic acid; cod liver oil, vitamin A, vitamin D, Eicosapentaenoic acid (EPA) and Docosahexaenoic acid (DHA), agent-Y.

40. The method of claim 39, wherein the administering is performed in an amount effective to increase activation of Nf-kB by 12.3 times (1231%) with respect to a baseline.

41. The method of claim 39, wherein the administering is performed in an amount effective to increase activation of Nf-kB with a maximum effect at 9 to 12 hours in epithelial cells and B-cells.

42. A method to provide a formulation capable of eliciting a biological response in an individual, the method comprising
providing one or more ethanolic plant extracts of claim 1 or 9; adding to the one or more ethanolic plant extracts at least one active to provide a candidate formulation; and testing the candidate formulation to detect a biological activity in vitro or in vivo.

43. The method of claim 42 wherein the at least one biologically active agents comprise at least one substance selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid DHA or a combination thereof

44. The method of claim 43 wherein the candidate formulation is a formulation comprising the one or more ethanolic plant extracts, at least one additional active agent selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid (DHA) or a combination thereof and at least one additional active agent.

45. A method to provide a formulation capable of eliciting a biological response in an individual the method comprising
providing at least one active agent selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid (DHA) or a combination thereof; adding to the at least one active agent to an ethanolic plant extract to provide a candidate formulation; and testing the candidate formulation to detect a biological activity in vitro or in vivo.

46. A system to provide a formulation having a biological activity, is the system comprising one or more ethanolic plant extracts of claim 1 or 9, and at least one biologically active agents selected from the group consisting of avocado oil, copper sulfate, ester-vitamin C, para aminobenzoic acid; cod liver oil, vitamin A, vitamin D, Eicosapentaenoic acid (EPA) and Docosahexaenoic acid DHA or a combination thereof, for combined use in providing a formulation having a biological activity in accordance with the disclosure.
